SEQUENCE LISTING (58 sequences)

SEQ ID NO: 1 - protein, 186 aa, Homo Sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 2 - protein, 8 aa, Artificial Sequence
  Humanized CDR sequence
GFTFNTYA

SEQ ID NO: 3 - protein, 10 aa, Artificial Sequence
  Humanized CDR sequence
IRSKYNNYAT

SEQ ID NO: 4 - protein, 16 aa, Artificial Sequence
  Humanized CDR sequence
VRHGNFGNSYVSWFAY

SEQ ID NO: 5 - protein, 125 aa, Artificial Sequence
  Antibody variable region
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAYWGQGTLVTVSS

SEQ ID NO: 6 - protein, 9 aa, Artificial Sequence
  Humanized CDR sequence
TGAVTTSNY

SEQ ID NO: 7 - protein, 9 aa, Artificial Sequence
  Humanized CDR sequence
ALWYSNLWV

SEQ ID NO: 8 - protein, 109 aa, Artificial Sequence
  Antibody variable region
QAVVTQEPSFSVSPGGTVTLTCRSSTGAVTTSNYANWVQQTPGQAFRGLIGGTNKRAPGVPARFSGSLIGDKAALTITGAQADDESIYFCALWYSNLWVFGGGTKLTVL

SEQ ID NO: 9 - protein, 8 aa, Artificial Sequence
  Humanized CDR sequence
GFTFNPYA

SEQ ID NO: 10 - protein, 125 aa, Artificial sequence
  Antibody variable region
EVKLVESGGGLVQPGGSLRLSCAASGFTFNPYAMNWVRQAPGKGLEWVARIRSKYNNYATYYADSVKDRFTISRDDSKSSLYLQMNNLKTEDTAMYYCVRHGNFGNSYVSWFAYWGQGTLVTVSS

SEQ ID NO: 11 - protein, 8 aa, Artificial Sequence
  Humanized CDR sequence
GFTFNMYA

SEQ ID NO: 12 - protein, 125 aa, Artificial Sequence
  Antibody variable domain
EVKLVESGGGLVQPGGSLRLSCAASGFTFNMYAMNWVRQAPGKGLEWVARIRSKYNNYATYYADSVKDRFTISRDDSKSSLYLQMNNLKTEDTAMYYCVRHGNFGNSYVSWFAYWGQGTLVTVSS

SEQ ID NO: 13 - protein, 10 aa, Artificial Sequence
  Humanized CDR sequence
IRSKYNEYAT

SEQ ID NO: 14 - protein, 125 aa, Artificial Sequence
  Antibody variable region
EVKLVESGGGLVQPGGSLRLSCAASGFTFNTYAMNWVRQAPGKGLEWVARIRSKYNEYATYYADSVKDRFTISRDDSKSSLYLQMNNLKTEDTAMYYCVRHGNFGNSYVSWFAYWGQGTLVTVSS

SEQ ID NO: 15 - protein, 16 aa, Artificial Sequence
  Humanized CDR sequence
VRGGNFGNSYVSWFAY

SEQ ID NO: 16 - protein, 125 aa, Artificial Sequence
  Antibody variable sequence
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRGGNFGNSYVSWFAYWGQGTLVTVSS

SEQ ID NO: 17 - protein, 16 aa, Artificial Sequence
  Humanized CDR sequence
VRNGNFGNSYVSWFAY

SEQ ID NO: 18 - protein, 125 aa, Artificial Sequence
  Antibody variable region
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRNGNFGNSYVSWFAYWGQGTLVTVSS

SEQ ID NO: 19 - protein, 16 aa, Artificial Sequence
  Humanized CDR sequence
VRHGNFPNSYVSWFAY

SEQ ID NO: 20 - protein, 125 aa, Artificial Sequence
  Antibody variable region
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFPNSYVSWFAYWGQGTLVTVSS

SEQ ID NO: 21 - protein, 16 aa, Artificial Sequence
  Humanized CDR sequence
VRHGNFGNSYVAWFAY

SEQ ID NO: 22 - protein, 125 aa, Artificial Sequence
  Antibody variable region
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVGWFAYWGQGTLVTVSS

SEQ ID NO: 23 - protein, 16 aa, Artificial Sequence
  Humanized CDR sequence
VRHGNFGNSYVGWFAY

SEQ ID NO: 24 - protein, 125 aa, Artificial Sequence; identical to SEQ ID NO: 22
  Antibody variable region

SEQ ID NO: 25 - protein, 16 aa, Artificial Sequence
  Humanized CDR sequence
VRHGNFGNSYVSWFAV

SEQ ID NO: 26 - protein, 125 aa, Artificial Sequence
  Antibody variable region
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAVWGQGTLVTVSS

SEQ ID NO: 27 - protein, 16 aa, Artificial Sequence
  Humanized CDR sequence
VRHGNFGNSYVSWFAM

SEQ ID NO: 28 - protein, 125 aa, Artificial Sequence
  Antibody variable domain
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAMWGQGTLVTVSS

SEQ ID NO: 29 - protein, 16 aa, Artificial Sequence
  Humanized CDR sequence
VRHGNFGNSYVSWFAR

SEQ ID NO: 30 - protein, 125 aa, Artificial Sequence
  Antibody variable domain
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFARWGQGTLVTVSS

SEQ ID NO: 31 - protein, 107 aa, Homo Sapiens [sequence omitted]

SEQ ID NO: 32 - protein, 106 aa, Homo Sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 33 - protein, 329 aa, Homo Sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 20 aa, Artificial Sequence
  Peptide containing epitope
AVGALKVPRNQDWLGVPRQL

SEQ ID NO: 35 - protein, 9 aa, Artificial Sequence
  Epitope peptide
KVPRNQDWL

SEQ ID NO: 36 - protein, 30 aa, Gallus gallus
SMLVLLPDEVSGLEQLESIINFEKLTEWTS

SEQ ID NO: 37 - protein, 7 aa, Gallus gallus
SIINFEK

SEQ ID NO: 38 - protein, 169 aa, Mus musculus
QDDAENIEYKVSISGTSVELTCPLDSDENLKWEKNGQELPQKHDKHLVLQDFSEVEDSGYYVCYTPASNKNTYLYLKARVCEYCVEVDLTAVAIIIIVDICITLGLLMVIYYWSKNRKAKAKPVTRGTGAGSRPRGQNKERPPPVPNPDYEPIRKGQRDLYSGLNQRAV

SEQ ID NO: 39 - protein, 8 aa, Mus musculus
GFTFSGYG

SEQ ID NO: 40 - protein, 8 aa, Mus musculus
ITSSSINI

SEQ ID NO: 41 - protein, 9 aa, Mus musculus
ARFDWDKNY

SEQ ID NO: 42 - protein, 116 aa, Mus musculus
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSS

SEQ ID NO: 43 - protein, 6 aa, Mus musculus
QDISNY

SEQ ID NO: 44 - protein, 9 aa, Mus musculus
QQYYNYPWT

SEQ ID NO: 45 - protein, 107 aa, Mus musculus
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIK

SEQ ID NO: 46 - protein, 8 aa, Mus musculus
GFNIKDYF

SEQ ID NO: 47 - protein, 8 aa, Mus musculus
INPDNGNT

SEQ ID NO: 48 - protein, 14 aa, Mus musculus
TRRDYTYEKAALDY

SEQ ID NO: 49 - protein, 121 aa, Mus musculus
EVQLQQSGAELVRPGALVKLSCKTSGFNIKDYFLHWVRQRPDQGLEWIGWINPDNGNTVYDPKFQGTASLTADTSSNTVYLQLSGLTSEDTAVYFCTRRDYTYEKAALDYWGQGASVIVSS

SEQ ID NO: 50 - protein, 6 aa, Mus musculus
GNIYNY

SEQ ID NO: 51 - protein, 9 aa, Mus musculus
QHFWSLPFT

SEQ ID NO: 52 - protein, 107 aa, Mus musculus
AIQMSQSPASLSASVGETVTITCRASGNIYNYLAWYQQKQGKSPHLLVYDAKTLADGVPSRFSGSGSGTQYSLKISSLQTEDSGNYYCQHFWSLPFTFGSGTKLEIK

SEQ ID NO: 53 - protein, 330 aa, Mus musculus
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPMVTCVVVDVSEDDPDVQISWFVNNVEVLTAQTQTHREDYNSTLRVVSALPIQHQDWMSGKEFKCKVNNKALPAPIERTISKPKGSVRAPQVYVLPPPEEEMTKKQVTLTCMVKDFMPEDIYVEWTNNGKTELNYKNTEPVLDSDGSYFMYSRLRVEKKNWVERNSYSCSVVHEGLHNHHTTKSFSRTPGK

SEQ ID NO: 54 - protein, 330 aa, Mus musculus
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPMVTCVVVDVSEDDPDVQISWFVNNVEVLTAQTQTHREDYNSTLRVVSALPIQHQDWMSGKEFKCKVNNKALPAPIERTISKPKGSVRAPQVYVLPPPEEEMTKKQVTLTCMVTDFMPEDIYVEWTNNGKTELNYKNTEPVLDSDGSYLMYSKLTVEKKNWVERNSYSCSVVHEGLHNHHTTKSFSRTPGK

SEQ ID NO: 55 - protein, 133 aa, Homo Sapiens [sequence omitted]

SEQ ID NO: 56 - protein, 114 aa, Homo Sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 57 - protein, 18 aa, Artificial sequence
  Synthetic peptide
KAGGKILTFDQLALESPK

SEQ ID NO: 58 - protein, 21 aa, Artificial sequence
  Synthetic peptide
VITGIKAVYNFATCGIFALIS

(19) United States
(12) Patent Application Publication
(10) Pub. No.: US 2023/0310599 A1
(43) Pub. Date: Oct. 5, 2023

(54) ANTIBODY THERAPY

(71) Applicant: GENMAB A/S, Copenhagen V (DK)

(72) Inventors: Hreinn BENONISSON, Äkarp (SE); Jim MIDDELBURG, Voorschoten (NL); Thorbald VAN HALL, Bodegraven (NL); Isil ALTINTAS, Utrecht (NL); Kristel KEMPER, Utrecht (NL); Janine SCHUURMAN, Utrecht (NL); Katy Ann LLOYD, Utrecht (NL); Vitalijs OVCINNIKOVS, Utrecht (NL); Gijsbertus ZOM, Utrecht (NL)

(21) Appl. No.: 18/023,808

(22) PCT Filed: Sep. 2, 2021
(86) PCT No.: PCT/EP2021/074314

§ 371 Date: Feb. 28, 2023

(30) Foreign Application Priority Data
Sep. 2, 2020 (EP) 20194018.6

(51) Int. Cl. A61K 39/42; A61P 35/00; A61P 37/04

(52) U.S. Cl. CPC A61K 39/42; A61P 35/00; A61P 37/04; A61K 2039/545

(57) ABSTRACT

The present invention relates to combination therapy within the field of oncology; in particular to combination therapy with vaccines and binding agents binding to CD3 and to a target antigen on tumor cells.

### FIELD OF INVENTION

The present invention relates to cancer therapy combining treatment with an immunogenic composition comprising at least one vaccine antigen, and a binding agent comprising an antigen-binding region that binds to CD3 and one that binds to a target antigen on tumor cells.

### BACKGROUND OF THE INVENTION

Advances in immunotherapy have transformed the field of cancer therapy. Aside from classical chemotherapy and irradiation, patients in a wide variety of cancer types have gained access to therapeutic options such as monoclonal antibody (mAb) treatment and adoptive cell transfer (ACT). Monospecific monoclonal antibodies can specifically and bivalently bind to an antigen expressed on a cell, e.g. a tumor-associated antigen or a T-cell-specific target. Bispecific antibodies (bsAbs) however can bind monovalently to two different targets, allowing targeting of two epitopes on one cell or two antigens on different cells. In the latter situation, a bsAb essentially brings two cells in close proximity, e.g. T cells to tumor cells. BsAbs containing a T-cell-specific arm and a tumor-associated antigen-specific arm, also known as T-cell engaging bsAbs or CD3 bsAbs, can crosslink T cells with tumor cells, thereby inducing T-cell mediated kill of the tumor cells, regardless of the TCR specificity of the T cells. ACT comprises the adoptive transfer of immune cells, mostly commonly T cells, into a cancer patient. In particular, two therapies using chimeric antigen receptor (CAR) T cells, which are genetically engineered to express an artificial high-affinity T-cell receptor, have been approved by the FDA for the treatment of hematological cancers (Schuster et al. N Engl J Med 2019; 380:45-56, Neelapu et al. N Engl J Med 2017; 377:2531-2544). Prophylactic vaccines are often aimed at inducing antibody responses against infectious agents to prevent disease. Therapeutic cancer vaccines, on the other hand, may be applied to boost existing tumor-specific T cells, e.g. by injecting peptides harboring tumor-specific sequences (Zwaveling et al., J Immunol. 2002; 169(1):350-8).

Implementation of these therapies has resulted in a better overall survival in many cancer types, although not all cancer patients benefit from immunotherapy. Notably, while T-cell engaging bsAbs have shown promising clinical results in hematological tumors, no CD3 bsAbs have been approved yet for the treatment of solid tumors, as clinical efficacy has not been demonstrated so far (Yu et al. J Hematol Oncol. 2017; 10: 155). Immunotherapies are notably less efficacious in so-called ‘cold’ tumors, which are tumors that are devoid of any (functional) immune infiltrate. Also, an immunosuppressive tumor microenvironment, characterized by the presence of suppressive immune cells such as regulatory T cells (Tregs), myeloid-derived suppressor cells (MDSCs) and/or M2-type macrophages (Xiong et al. Adv Biol 2021; 5(3):e1900311), may prevent T-cell infiltration or locally suppress T-cell function. In order to convert this suppressive environment, intratumoral injection of (non-adjuvanted) seasonal influenza vaccine (Newman et al, PNAS 2020), genetically engineered murine cytomegalovirus (Erkes et al, Molecular Therapy 2016), or inactivated modified vaccinia virus Ankara (Dai et al, Science Immunol 2017) have been studied, which all resulted in enhanced anti-tumor efficacy in murine tumor models. Mechanistically, such therapies are hypothesized to act via triggering of pattern-recognition receptors expressed by tumor-resident antigen-presenting cells, leading to a conversion of the immunosuppressive tumor microenvironment into an environment supporting immune infiltration and T-cell activation. The I clinical use of Bacillus Calmette-Guerin (BCG) immunotherapy to treat non-muscle-invasive bladder cancer is also contributed to by the triggering of local inflammation and immune cell infiltration in tumors (Redelman-Sidi et al. Nat Rev Urol 2014; 11(3):153-62).

Increasing the number of tumor-infiltrating T cells has been the aim of many therapeutic vaccination studies. However, while therapeutic vaccination showed promising results in pre-malignant lesions, such vaccinations did not show efficient clinical efficacy in cancer patients. Therefore, combinatorial therapies may be more efficacious (Melief et al., J Clin Invest. 2015; 125(9): 3401-3412). This was also concluded by Benonisson et al., who demonstrated that treatment of mice bearing a murine melanoma (B16F10) with a murine T-cell engaging bsAb resulted in enhanced tumor T-cell infiltration and activation, although no T-cell immunity was installed (Benonisson et al., Mol Cancer Ther. 2019 February; 18(2):312-322). Both US2015095811 and US20190382490 describe combination therapies consisting of a cancer-specific vaccine and immune checkpoint inhibition through monoclonal antibodies. Furthermore, Ly et al. investigated a combination therapy consisting of ACT, tumor-specific peptide vaccination, adjuvant Toll-like receptor-ligand (TLR-L)7 and IL-2, and found strongly increased T-cell numbers that could mediate tumor eradication (Ly et al. Cancer Res. 2010; 70(21):8339-46).

There remains a need for methods that increase the number of tumor-infiltrating T cells in solid tumors, which can subsequently be exploited for combination therapy with T-cell engaging bsAb antibody treatment. In particular, there remains a need for therapies comprising a T-cell engaging bsAb and a tumor-non-specific vaccine, which would allow for the administration of a generic vaccine to a patient irrespective of their specific cancer type.

### SUMMARY OF INVENTION

It is an object of the present invention to provide a method for preventing or reducing growth of a tumor, or for treatment of cancer in a subject in need thereof, comprising providing to the subject
i) a binding agent comprising an antigen-binding region that binds to CD3, such as human CD3, and an antigen-binding region that binds to a target antigen on cells of said tumor or cancer; and
ii) an immunogenic composition comprising at least one vaccine antigen.

In a 2^{nd} aspect, the present invention relates to a method for preventing or reducing growth of a tumor, or for treatment of cancer in a subject in need thereof, comprising providing to the subject
i) A nucleic acid construct encoding a binding agent comprising a 1^{st} binding region that binds to CD3 and a 2^{nd} binding region that binds to a target antigen on cells of said tumor or cancer; and
ii) an immunogenic composition comprising at least one vaccine antigen.

In a 3^{rd} aspect, the present invention relates to a binding agent for use in preventing or reducing tumor outgrowth or treatment of cancer in a subject, the binding agent comprising a binding region that binds to CD3 and a binding region that binds to a target on cells of said tumor or cancer, wherein the use comprises providing the antibody to the subject in combination with an immunogenic composition comprising at least one vaccine antigen.

In a 4^{th} aspect, the present invention relates to an immunogenic composition comprising at least one vaccine antigen for use in preventing or reducing tumor outgrowth or treatment of cancer in a subject, wherein the immunogenic composition is provided to the subject in combination with a binding agent comprising a binding region that binds to CD3 and a binding region that binds to a target on cells of said tumor or cancer.

In a 5^{th} aspect, the present invention relates to use of a binding agent in the manufacture of a medicament for treatment of cancer in a subject, the binding agent comprising a binding region that binds to CD3 and a binding region that binds to a target on cells of said tumor or cancer, wherein the treatment comprises providing said binding agent to the subject in combination with an immunogenic composition comprising at least one vaccine antigen.

In a 6^{th} aspect, the invention provides use of an immunogenic composition comprising at least one vaccine antigen in the manufacture of a medicament for treatment of cancer in a subject, wherein the immunogenic composition is provided to the subject in combination with a binding agent comprising a binding region that binds to CD3 and a binding region that binds to a target on cells of said tumor or cancer.

Finally, the invention also provides a kit of parts comprising a binding agent comprising a binding region that binds to CD3 and a binding region that binds to a target on cells of a tumor or cancer, and an immunogenic composition comprising at least one vaccine antigen.

These and other aspects and embodiments are described in more detail in the following sections.

### BRIEF DESCRIPTION OF THE FIGURES

FIG. 1. The figure shows the effect of CXCR3-mediated immune cell infiltration on CD3×TA99 bispecific antibody treatment. (A) Treatment timeline. On day 0, WT or CXCR3 knockout (KO) C57BL/6 mice were injected s.c. with 50,000 syngeneic B16F10 melanoma cells. On day 6 and 9, mice were injected i.p. with 12.5 μg CD3×TA99 bsAb. Tumor outgrowth was monitored by measuring tumors twice weekly and sacrificing when tumor volumes exceeded 1000 mm³. (B) Tumor growth curves of the individual mice (n=6-10). (C) Kaplan-Meier plot showing survival of mice within the treatment groups. Statistically significant differences in survival were calculated by Mantel-Cox analysis. (D) Following the same treatment timeline as presented in (A) in a separate experiment, mice (n=4-5) were sacrificed on day 20, tumors and spleens were processed for analysis of immune infiltrate. Total CD45 positive cell infiltration and proportions are shown of T cells and NK cells measured by flow cytometry. (E-G) Effect of CXCR3-mediated endogenous cell infiltration on OT1 ACT+OVA vaccination in combination with CD3×TA99 bispecific treatment. (E) Treatment timeline. On day 0, CXCR3 knockout C57BL/6 mice were injected s.c. with 50,000 B16F10 tumor cells. Mice received 1×10⁶ OT1 enriched-splenocytes on day 3. On day 3 and 10, the mice were immunized with the OVA peptide. As adjuvant to the immunizations, Aldara was applied topically at the injection site on day 3 and 10, and recombinant human IL-2 was injected i.p. on day 10 and 11. On day 12 and day 15, the mice were injected i.p. with 12.5 μg CD3×TA99 bsAb. Tumor growth was monitored three times weekly and mice were sacrificed when tumor volumes exceeded 1000 mm³. (F) Tumor growth curves of the individual mice. (G) The percentage of surviving mice within the treatment groups. In (F) and (G) untreated control mice from (A) are shown for reference. Statistically significant differences in survival were calculated by Mantel-Cox analysis. ns=not significant, * P<0.05

FIG. 2: C57BL/6 mice were injected s.c. with 100,000 syngeneic B16F10 melanoma cells at day 0. On day 5, 3×10⁶ Pmel-1 T cells were administered i.v. At days 6 and 9, mice were injected i.p. with 12.5 μg CD3×TA99 bsAb. On days 6 and 13, the mice were immunized s.c. with the KVP peptide. As adjuvant to the immunizations, Aldara was applied topically at the injection site. In addition, recombinant human IL-2 was injected i.p. on days 13 and 14. Tumor growth was monitored twice weekly and mice were sacrificed when tumor volumes exceeded 1000 mm³. (A) Schematic overview of the experiment. (B) Tumor growth curves of the individual mice. (C) The percentage of surviving mice within the treatment groups was plotted and compared to the survival of the untreated mice (** P<0.01, *** P<0.001). Statistically significant differences in survival were calculated by Mantel-Cox analysis.

FIG. 3. On day −1, C57BL/6 mice were administered i.v. with 1×10⁶ of the enriched OT1 T cells. On day 0, they were injected s.c. with 100,000 syngeneic B16F10 melanoma cells. On day 3 and 10, the mice were immunized with the KVP or OVA peptide. As adjuvant, Aldara was applied topically at the injection site on days 3 and 10, and recombinant human IL-2 was injected i.p. on day 10 and 11. On day 12 and 15, the mice were injected i.p. with 12.5 μg CD3×TA99 bsAb. Tumor growth was monitored twice weekly and mice were sacrificed when tumor volumes exceeded 1000 mm³. (A) Schematic overview of the experiment. (B) Tumor growth curves of the individual mice. (C) The percentage of surviving mice within the treatment groups was plotted and compared to the survival of the untreated mice (* P<0.05). Statistically significant differences in survival were calculated by Mantel-Cox analysis.

FIG. 4. The figure shows that a combination of CD3×TA99 bsAb and tumor-specific vaccination or a combination of CD3×TA99 bsAb and tumor-nonspecific vaccination combined with ACT of matching, tumor-nonspecific T cells similarly enhance anti-tumor efficacy. (A) On day −1, 1×10⁶ of the enriched OT-1 T cells were administered i.v. to C57BL/6 mice. On day 0, they were injected s.c. with 100,000 murine B16F10 melanoma cells. On day 3 and 10, the mice were immunized with the KVP or OVA peptide. As adjuvant, Aldara was applied topically at the injection site on days 3 and 10, in addition to recombinant human IL-2 i.p. injections on day 10 and 11. On day 12 and 15, the mice were injected i.p. with 12.5 μg CD3×TA99 bsAb. Tumor growth was monitored twice weekly. (B) Tumor growth curves for all treatment groups. (C) Kaplan-Meier plot showing survival of mice in the different treatment groups. Mice were sacrificed when tumor volumes exceeded 1000 mm³ and the percent of surviving mice within the treatment groups was plotted and compared to the survival of the untreated mice (** P<0.01). Statistically significant differences in survival were calculated by Mantel-Cox analysis.

FIG. 5. The figure shows OT1 T-cell infiltration and activation in KPC3-TRP1-positive and KPC3-TRP1-negative tumors in albino C57BL/6 mice treated with combinations of OT1 T cell ACT, OVA peptide vaccination and CD3×TA99 bsAb. (A) Treatment timeline. On day 0, C57BL/6 mice were injected s.c. with 80,000 KPC3 tumors cells on both the left side (TRP1-negative KPC3 cells) and right side of the back (TRP1-positive KPC3 cells). On day 5, 1×10⁶ of the enriched TbiLuc×OT1 splenocytes were administered i.v. to all mice, while the mice receiving OVA vaccination were injected s.c. with an ovalbumin-derived synthetic long peptide mixed with CpG ODN1826 on day 6 and 13. The CD3×TA99 bsAb was injected i.p. on day 15 and 19 (indicated with arrows). T-cell activation of OT1 T cells, measured as Cycluc1 bioluminescent flux/second, is shown in groups of mice that received (B) OT1 T cell ACT and CD3×TA99 bsAb treatment, (C) OT1 T cell ACT and OVA vaccination, or (D) OT1 T cell ACT, CD3×TA99 bsAb treatment and OVA vaccination. T-cell infiltration of OT1 T cells, measured as D-luciferin bioluminescent flux/second, is shown in groups of mice that received (E) OT1 T cell ACT and CD3×TA99 bsAb treatment, (F) OT1 T cell ACT and OVA vaccination, or (G) OT1 T cell ACT, CD3×TA99 bsAb treatment and OVA vaccination. The same dataset is presented in (H-K), showing T-cell activation (H, I) and T-cell infiltration (J, K) in KPC3 tumors (H, J) and KPC3-TRP1 tumors (1, K). The CD3×TA99 bsAb injections on day 15 and 19 are indicated with arrows. (L) Treatment timeline. The schedule from day 0 to day 13 was identical to that described for (A). On day 16, mice were euthanized, and single cell suspensions of blood, spleen and tumors were prepared to analyze immune subset infiltration by flow cytometry. Percentages of OT1 T cells within CD45+ population and OT1 naïve (CD44-CD62L+) vs. effector (CD44+CD62L−) phenotype in the blood (M) and spleen (N), and TRP1-negative and TRP1-positive KPC3 tumors (O). (P) Treatment timeline. The schedule from day 0 to day 13 was identical to that described for (L). The CD3×TA99 bsAb was injected i.p. on day 16 and 19, and mice were euthanized on day 20. Percentages of OT1 T cells within CD45+ population and OT1 naïve (CD44-CD62L+) vs. effector (CD44+CD62L−) phenotype in the blood (Q) and spleen (R). (S) Left: percentage of OT1 T cells within the CD45+ population in TRP1-negative and TRP1-positive KPC3 tumors in mice receiving the indicated treatment. Middle: surface level expression of CD69 on OT1 T cells following the addition of the bsAb to the combination of OT1 T cell ACT and OVA vaccination. Right: percentages of OT1 T cells with an effector phenotype (CD44+CD62L−) within the CD45+ population. Statistical significance was determined using a Welch t-test; * p<0.05, **p<0.01, *** p<0.001.

FIG. 6. The figure shows the percentages of different immune cell subsets within B16F10 tumors in mice treated with either IL-2, Aldara cream or a combination of IL-2 and Aldara, as determined by flow cytometry. (A) Treatment timeline. (B) Percentages of CD45+ cells (immune cells) within the live cell gate of tumors derived from mice left untreated, treated with IL-2, Aldara or the combination of IL-2 and Aldara. (C) Percentages of conventional DCs (cDC1) within the CD45+ population in tumors from mice treated as indicated under (B). (D) Percentages of monocyte-derived DCs (MoDC) within the CD45+ population in tumors from mice treated as indicated under (B). (E) Percentages of NK1.1⁺/CD3⁻ (NK) cells within the CD45⁺ population in tumors from mice treated as indicated under (B). (F) Percentages of M1 type macrophages within the CD45⁺ population in tumors from mice treated as indicated under (B). (G) Percentages of resting macrophages (M0 macrophages) within the CD45⁺ population in tumors from mice treated as indicated under (B). (H) Percentages of immature macrophages within the CD45⁺ population in tumors from mice treated as indicated under (B). (I) Percentages of T cells within the CD45⁺ population in tumors from mice treated as indicated under (B). Statistical analysis performed by Mann Whitney test; * p<0.05.

FIG. 7: The figure shows that combining CD3×TA99 bsAb with a tumor-specific vaccination or a tumor-nonspecific vaccination similarly enhance anti-tumor efficacy. (A) Schematic overview of the experiment. On day 0, C57BL/6 mice were injected s.c. with 80,000 murine B16F10 melanoma cells. On day 3 and 10, the mice were immunized with the KVP or Rpl18 peptide. As adjuvant, Aldara was applied topically at the injection site on days 3 and 10, in addition to i.p. injections of recombinant human IL-2 on day 10 and 11. On day 12 and 15, the mice were injected i.p. with 12.5 μg CD3×TA99 bsAb. Tumor growth was monitored twice weekly. (B) Tumor growth curves of the individual mice. (C) Kaplan-Meier plot showing survival of mice in the different treatment groups. Mice were sacrificed when tumor volumes exceeded 1000 mm³ and the percent of surviving mice within the treatment groups was plotted and compared to the survival of the mice treated with CD3×TA99 bispecific only (*P<0.05, ** P<0.01). Statistically significant differences in survival were calculated by Mantel-Cox analysis.

FIG. 8: The figure shows the percentages of different immune cell subsets within B16F10 tumors in mice vaccinated with a tumor-nonspecific peptide (Rpl18), as determined by flow cytometry. (A) Treatment timeline. (B) Percentages of CD45⁺ cells (immune cells), CD8⁺ T cells, NK cells, NKT cells, ratio of CD8/CD4 T cells, ratio of CD8/Treg, memory phenotype (naïve T cells [CD44⁻CD62L⁺], effector T cells [Teff; CD44⁺CD62L⁻] and central memory T cells [Tcm; CD44⁺CD62L⁺]) of CD8⁺ T cells and CD4⁺ T cells, monocyte-derived DCs (MoDC), immature macrophages, eosinophils, neutrophils and conventional DCs (cDC1) within the live cell gate of tumors derived from mice left untreated or mice vaccinated with Rpl18 peptide, Aldara and IL-2. (C) Percentages of CD8⁺ T cells, CD4⁺ T cells, NK cells, NKT cells and CD19⁺ B cells positive for CD103, granzyme B (GzmB); Percentages of CD8⁺ T cells, CD4⁺ T cells, NK cells and NKT cells positive for PD-1, and NKG2A within tumor populations from mice treated as indicated under (B). Statistical analysis performed by Welch's t-test; * p<0.05, ** p<0.01, *** p<0.001.

FIG. 9. The figure shows the phenotyping of immune cells in tumors and spleens of KPC3-TRP1-carrying mice treated with CD3×TA99 bsAb alone or in combination with tumor non-specific vaccination. (A) Treatment timeline. On day 0, C57BL/6 mice were injected s.c. with 80,000 TRP1-positive KPC3 tumor cells. On day 8 and 15, mice in the vaccination group were immunized with 150 μg OVA peptide. As adjuvant, Aldara was applied topically at the injection site on days 8 and 15, in addition to 600.000 UI hIL-2 i.p. injections on day 15 and 16. On day 17, mice were injected i.p. with 12.5 μg CD3×TA99 bsAb. On day 19, mice were sacrificed and tumors and spleens were processed for analysis. Analysis of CD45⁺, T cell, T cell subset composition (CD8, CD4⁺FoxP3⁻, CD4⁺FoxP3⁺, naïve T cells [CD44⁻CD62L⁺], effector T cells [Teff; CD44⁺CD62L⁻] and central memory T cells [Tcm; CD44⁺CD62L⁺]), B cells and NK cells within tumor (B) and spleen (C). (D, E) Expression of activatory and inhibitory surface markers, effector molecules and transcription factors (4-1BB, CD27, OX40, GzmB, CD49a, CD40L, CD69, PD-1, TIGIT, Tim3, CD39, NKG2A, KLRG1, Ki67, TCF-1, Eomes, Tbet, GATA-3, and RorγT) in CD8⁺ T cells within tumor (D) and spleen (E). (F) Expression of activatory and inhibitory surface markers and effector molecules (GzmB, CD69, CD49a, CD39, KLRG1, NKG2A) on NK cells within the tumor. (G) Top: Frequency of macrophages within CD45⁺ cells in the tumor. Bottom: Distribution of iNOS-Egr2− (M0), iNOS+Egr2− (M1) and iNOS-Egr2+ (M2) macrophage phenotypes. Statistical analysis was performed using the Brown-Forsythe and Welch ANOVA and p-values determined with Dunnett's T3 multiple comparisons test; ns=not significant, * p<0.05, ** p<0.01, ***p<0.001, ****p<0.0001.

FIG. 10: (A) Schematic overview of the experimental timeline. On day −30, C57BL/6 mice were administered with 100×TCID50 of HK×31 influenza virus. On day 0, they were injected s.c. with 80,000 syngeneic B16F10 melanoma cells. On day 10, the mice were immunized with 2×10⁶ TCID50 heat-inactivated influenza virus. As adjuvant, Aldara was applied topically at the injection site on day 10, and recombinant human IL-2 was injected i.p. on day 10 and 11. On day 12 and day 15, the mice were injected i.p. with 12.5 μg CD3×TA99 bsAb. Mice were sacrificed when tumor volumes exceeded 1000 mm³ (B) Tumor growth curves of the individual mice in the different treatment groups. Tumor volume depicted as mm³. (C) Kaplan-Meier plot showing survival of mice within the treatment groups compared to the survival of the untreated mice (** P<0.01). Statistically significant differences in survival were calculated by Mantel-Cox analysis.

### DETAILED DESCRIPTION

### Definitions

The term “binding agent” in the context of the present invention refers to any agent capable of binding to desired antigens. In certain embodiments of the invention, the binding agent is an antibody, antibody fragment, or construct thereof. The binding agent may also comprise synthetic, modified or non-naturally occurring moieties, in particular non-peptide moieties. Such moieties may, for example, link desired antigen-binding functionalities or regions such as antibodies or antibody fragments. In one embodiment, the binding agent is a synthetic construct comprising antigen-binding complementarity determining regions (CDRs) or variable regions.

The term “immunoglobulin” refers to a class of structurally related glycoproteins consisting of two pairs of polypeptide chains, one pair of light (L) low molecular weight chains and one pair of heavy (H) chains, all four interconnected by disulphide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as VH) and a heavy chain constant region (abbreviated herein as CH). The heavy chain constant region typically is comprised of three domains: CH1, CH2, and CH3. The hinge region is the region between the CH1 and CH2 domains of the heavy chain and is highly flexible. Disulphide bonds in the hinge region are part of the interactions between two heavy chains in an IgG molecule. Each light chain typically is comprised of a light chain variable region (abbreviated herein as VL) and a light chain constant region (abbreviated herein as CL). The VH and VL regions may be further subdivided into regions of hypervariability (or hypervariable regions which may be hypervariable in sequence and/or in the form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)). Unless otherwise stated or contradicted by context, CDR sequences herein are identified according to IMGT rules using DomainGapAlign (Lefranc M P., Nucleic Acids Research 1999; 27:209-212 and Ehrenmann F., Kaas Q. and Lefranc M.-P. Nucleic Acids Res., 38, D301-307 (2010); see also internet http address www.imgt.org/). Unless otherwise stated or contradicted by context, reference to amino acid positions in the constant regions in the present invention is according to the Eu-numbering (Edelman et al., Proc Natl Acad Sci USA. 1969 May; 63(1):78-85; Kabat et al., Sequences of Proteins of Immunological Interest, Fifth Edition. 1991 NIH Publication No. 91-3242). For example, SEQ ID NO: 33 herein sets forth amino acid positions 118-446 according to Eu numbering, of the IgG1m(f) heavy chain constant region.

The term “amino acid” and “amino acid residue” may herein be used interchangeably, and are not to be understood limiting. Amino acids are organic compounds containing amine (—NH₂) and carboxyl (—COOH) functional groups, along with a side chain (R group) specific to each amino acid. In the context of the present invention, amino acids may be classified based on structure and chemical characteristics. Thus, classes of amino acids may be reflected in one or both of the following tables:

TABLE 1
Main classification based on structure and general
chemical characterization of R group

| Class | Amino acid |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 2
Alternative Physical and Functional Classifications
of Amino Acid Residues

| Class | Amino acid |
|---|---|
| Hydroxyl group containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, |
| | W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn | A, C, D, E, G, H, K, N, Q, R, S, |
| formation | P, and T |
| Flexible residues | Q, T, K, S, G, P, D, E, and R |

Substitution of one amino acid for another may be classified as a conservative or non-conservative substitution. In the context of the invention, a “conservative substitution” is a substitution of one amino acid with another amino acid having similar structural and/or chemical characteristics, such as a substitution of one amino acid residue for another amino acid residue of the same class as defined in any of the two tables above: for example, leucine may be substituted with isoleucine as they are both aliphatic, branched hydrophobes. Similarly, aspartic acid may be substituted with glutamic acid since they are both small, negatively charged residues.

The term “amino acid corresponding to position . . . ” as used herein refers to an amino acid position number in a human IgG1 heavy chain. Corresponding amino acid positions in other immunoglobulins may be found by alignment with human IgG1. Thus, an amino acid or segment in one sequence that “corresponds to” an amino acid or segment in another sequence is one that aligns with the other amino acid or segment using a standard sequence alignment program such as ALIGN, ClustalW or similar, typically at default settings, provided that the other sequence has at least 50%, at least 80%, at least 90%, or at least 95% identity to a human IgG1 heavy chain. It is considered well-known in the art how to align a sequence or segment in a sequence and thereby determine the corresponding position in a sequence to an amino acid position according to the present invention.

The term “antibody” (Ab) in the context of the present invention refers to an immunoglobulin molecule, a fragment of an immunoglobulin molecule, or a derivative of either thereof, which has the ability to specifically bind to an antigen under typical physiological conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The term antibody when used herein comprises not only monospecific antibodies, but also multispecific antibodies which comprise multiple, such as two or more, e.g. three or more, different antigen-binding regions. The constant regions of the antibodies (Abs) may mediate the binding of the immunoglobulin to host tissues or factors, including various cells of the immune system (such as effector cells) and components of the complement system such as C1q, the first component in the classical pathway of complement activation. As indicated above, the term antibody herein, unless otherwise stated or clearly contradicted by context, includes fragments of an antibody that are antigen-binding fragments, i.e., that retain the ability to specifically bind to the antigen. It has been shown that the antigen-binding function of an antibody may be performed by fragments of a full-length antibody. Examples of antigen-binding fragments encompassed within the term “antibody” include (i) a Fab′ or Fab fragment, a monovalent fragment consisting of the VL, VH, CL and CH1 domains, or a monovalent antibody as described in WO2007059782 (Genmab); (ii) F(ab′)₂ fragments, bivalent fragments comprising two Fab fragments linked by a disulfide bridge at the hinge region; (iii) an Fd fragment consisting essentially of the VH and CH1 domains; (iv) an Fv fragment consisting essentially of the VL and VH domains of a single arm of an antibody, (v) dAb fragments (Ward et al., Nature 341, 544-546 (1989)), which consist essentially of a VH domain and are also called domain antibodies (Holt et al; Trends Biotechnol. 2003 November; 21(11):484-90); (vi) camelid or Nanobody molecules (Revets et al; Expert Opin Biol Ther. 2005 January; 5(1):111-24) and (vii) an isolated CDR. Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single chain antibodies are encompassed within the term antibody unless otherwise noted or clearly indicated by context. Although such fragments are generally included within the meaning of antibody, they collectively and each independently are unique features of the present invention, exhibiting different biological properties and utility. It also should be understood that the term antibody, unless specified otherwise, also includes polyclonal antibodies, monoclonal antibodies (mAbs), antibody-like polypeptides, such as chimeric antibodies and humanized antibodies, and antibody fragments retaining the ability to specifically bind to the antigen (antigen-binding fragments) provided by any known technique, such as enzymatic cleavage, peptide synthesis, and recombinant techniques. An antibody as generated can be of any isotype. The term “isotype” as used herein, refers to the immunoglobulin (sub)class (for instance IgG (including subclasses IgG1, IgG2, IgG3, IgG4), IgD, IgA, IgE, or IgM) or any allotype thereof, such as IgG1m(za) and IgG1m(f) [SEQ ID NO: 33) that is encoded by heavy chain constant region genes. Thus, in one embodiment, the antibody comprises a heavy chain of an immunoglobulin of the IgG1 class or any allotype thereof. Further, each heavy chain isotype can be combined with either a kappa (κ) or lambda (λ) light chain.

Hence, when a particular isotype, e.g. IgG1, is mentioned herein, the term is not limited to a specific isotype sequence, e.g. a particular IgG1 sequence, but is used to indicate that the antibody is closer in sequence to that isotype, e.g. IgG1, than to other isotypes. Thus, e.g. an IgG1 antibody of the invention may be a sequence variant of a naturally-occurring IgG1 antibody, including variations in the constant regions.

The term “multispecific antibody” in the context of the present invention refers to an antibody having two or more different antigen-binding regions defined by different antibody sequences. When the antibody has two different antigen-binding regions defined by different antibody sequences it is referred to as a “bispecific antibody”. A multispecific antibody can be of any format, including any bispecific antibody format.

The term “human antibody”, as used herein, is intended to include antibodies having variable and framework regions derived from human germline immunoglobulin sequences and a human immunoglobulin constant domain. The human antibodies of the invention may include amino acid residues not encoded by human germline immunoglobulin sequences (e.g., mutations, insertions or deletions introduced by random or site-specific mutagenesis in vitro or by somatic mutation in vivo). However, the term “human antibody”, as used herein, is not intended to include antibodies in which CDR sequences derived from the germline of another non-human species, such as a mouse, have been grafted onto human framework sequences.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody CDRs, which together form the antigen binding site, onto a homologous human acceptor FR (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

When used herein, unless contradicted by context, the term “Fc region” refers to an antibody region consisting of the two Fc sequences of the heavy chains of an immunoglobulin, wherein said Fc sequences comprise, in the direction from the N- to C-terminal end of the antibody, at least a hinge region, a CH2 domain, and a CH3 domain. An Fc region of the antibody may mediate the binding of the immunoglobulin to host tissues or factors, including various cells of the immune system (such as effector cells) and components of the complement system.

The term “hinge region” as used herein refers to the hinge region of an immunoglobulin heavy chain. Thus, for example the hinge region of a human IgG1 antibody corresponds to amino acids 216-230 according to the Eu numbering as set forth in Kabat (Kabat, E. A. et al., Sequences of proteins of immunological interest. 5th Edition—US Department of Health and Human Services, NIH publication No. 91-3242, pp 662,680,689 (1991)). However, the hinge region may also be any of the other subtypes as described herein.

The term “CH1 region” or “CH1 domain” as used herein refers to the CH1 region of an immunoglobulin heavy chain. Thus, for example the CH1 region of a human IgG1 antibody corresponds to amino acids 118-215 according to the Eu numbering as set forth in Kabat (ibid). However, the CH1 region may also be any of the other subtypes as described herein.

The term “CH2 region” or “CH2 domain” as used herein refers to the CH2 region of an immunoglobulin heavy chain. Thus, for example the CH2 region of a human IgG1 antibody corresponds to amino acids 231-340 according to the Eu numbering as set forth in Kabat (ibid). However, the CH2 region may also be any of the other subtypes as described herein.

The term “CH3 region” or “CH3 domain” as used herein refers to the CH3 region of an immunoglobulin heavy chain. Thus for example the CH3 region of a human IgG1 antibody corresponds to amino acids 341-447 according to the Eu numbering as set forth in Kabat (ibid). However, the CH3 region may also be any of the other subtypes as described herein.

The term “full-length” when used in the context of an antibody indicates that the antibody is not a fragment, but contains all of the domains of the particular isotype normally found for that isotype in nature, e.g. the VH, CH1 region, CH2 region, CH3 region, hinge, VL and CL domains for an IgG1 antibody.

As used herein, the terms “binding” or “capable of binding” in the context of the binding of an antibody to a predetermined antigen or epitope typically is a binding with an affinity corresponding to a K_{D} of about 10⁻⁷ M or less, such as 10⁻⁷ M or less, such as about 10⁻⁸ M or less, such as 10⁻⁸ M or less, such as about 10⁻⁹ M or less, such as 10⁻⁹ M or less, such as about 10⁻¹⁰ M or less, such as 10⁻¹⁰ M or less, such as about 10⁻¹¹ or such as 10⁻¹¹ M or even less, when determined using Bio-Layer Interferometry (BLI) or, for instance, when determined using surface plasmon resonance (SPR) technology using the antigen as the ligand and the antibody as the analyte. The antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} for binding to a non-specific antigen (e.g., bovine serum albumin, casein) other than the predetermined antigen or a closely-related antigen. The amount with which the affinity is higher is dependent on the K_{D} of the antibody, so that when the K_{D} of the antibody is very low (that is, the antibody is highly specific), then the degree to which the affinity for the antigen is lower than the affinity for a non-specific antigen may be at least 10,000-fold.

The term “k_{d}” (sec⁻¹), as used herein, refers to the dissociation rate constant of a particular antibody-antigen interaction. Said value is also referred to as the k_{off} value.

The term “K_{D}” (M), as used herein, refers to the dissociation equilibrium constant of a particular antibody-antigen interaction.

The term “binding region” as used herein, refers to a region of a binding agent, such as an antibody, which is capable of binding to a molecule, such as a polypeptide, e.g. present on a cell, bacterium, or virion. When used herein, “binding region” may in particular refer to the region of a binding agent, such as an antibody, which interacts with an antigen and comprises both a heavy chain variable (VH) region and a light chain variable (VL) region.

The term “antigen” as used herein refers broadly to any substance that is capable of eliciting an immune response. In some embodiments an “antigen” may be proteinaceous or may be a polysaccharide.

The term “target antigen” as used herein is a molecule capable of being bound by an (therapeutic) antibody.

The term “immunogenic composition”, as used herein, refers to a composition containing an antigen and/or at least one nucleic acid molecule encoding an antigen, with or without an immunostimulatory agent such as an adjuvant or a molecule used to increase the immune response to the antigen. An immunogenic composition would be understood by one of skill in the art, to be a composition which upon administration to a human or animal subject would elicit a humoral response (e.g. antibody response), cellular response (e.g. T-cell response), or an innate response (e.g. activation of granulocytes, antigen presenting cells, NK cells) and/or local inflammation, within said animal or human subject.

A “vaccine” as used herein is understood to be a preparation comprising at least one vaccine antigen and/or at least one nucleic acid molecule encoding a vaccine antigen, and a pharmaceutically acceptable diluent or carrier, optionally in combination with excipient, adjuvant and/or additive or protectant. The vaccine antigen may be derived from any material that is suitable for vaccination, e.g., the antigen or immunogen may be derived from a pathogen, such as from bacteria or virus particles etc., or from a tumor or cancerous tissue. The antigen or immunogen stimulates the body's adaptive immune system to provide an adaptive immune response, such as a humoral response (e.g. antibody response) and/or cellular response (e.g. T-cell response), either in the presence or absence of an adjuvant. In the context of the present invention, the vaccine may be a therapeutic vaccine, which is given to a subject after infection or after the subject has been diagnosed with a tumor or cancer, and is intended to reduce or arrest disease progression. The vaccine may also be a preventive or prophylactic vaccine, which is provided to the subject prior to exposure to an infectious agent or before the subject has been diagnosed with a tumor or cancer, and is intended to prevent initial infection or tumorigenesis or reduce the rate or burden of the infection or tumor growth.

As used herein, the term “vaccine antigen” is a molecule, which is capable of inducing a humoral immune response and/or a cellular immune response leading to the production of B- and/or T-lymphocytes specific to the said antigen.

The term “epitope”, as used herein, refers to a portion of an antigen that is recognized by an antibody and/or by the immune system of a subject, specifically by antibodies, B cells, or T cells. Epitopes include B-cell epitopes and T-cell epitopes. B-cell epitopes are peptide sequences or carbohydrates which are required for recognition by specific antibody producing B cells. B-cell epitopes refer to a specific region of the antigen that is recognized by an antibody. The portion of an antibody that binds to the epitope is called a paratope. An epitope may be a conformational epitope or a linear epitope, based on the structure and interaction with the paratope. A linear, or continuous, epitope is defined by the primary amino acid sequence of a particular region of a protein, or can be defined by monosaccharide composition. The sequences that interact with the antibody are situated next to each other sequentially on the protein or carbohydrate, and the epitope can usually be mimicked by a single peptide. Conformational epitopes are epitopes that are defined by the conformational structure of the native protein, carbohydrate or glycoprotein. These epitopes may be continuous or discontinuous, i.e. components of the epitope can be situated on disparate parts of the protein, which are brought close to each other in the folded native protein structure. T-cell epitopes are composed of peptide sequences, lipids, carbohydrates or metabolites which, in association with proteins on the cell surface are required for recognition by specific T cells. T-cell epitopes are derived from polypeptides, lipids, carbohydrates or metabolites, which are processed intracellularly by APCs and presented on the surface of all nucleated cells, including antigen-presenting cells (APCs), where they are bound to classical major histocompatibility complex (MHC) molecules including MHC class I and MHC class II., or non-classical MHC-like molecules such as MR1, CD1 or butrophilin (BTN3A1). A T-cell epitope presented in the context of an MHC class I or MHC class II molecule can be recognized by the T-cell receptor of a specific T cell.

As used herein a “live vaccine” refers to a vaccine comprising live microorganisms or viruses. Most live vaccines are “live attenuated vaccines” prepared from live microorganisms or viruses, which have either been selected for not being virulent or for having reduced virulence in the relevant subject, or have been subject to treatment to reduce or abolish virulence, while retaining the ability to induce an immune response, such as protective immunity. The treatment may for instance comprise culturing under adverse conditions, leading to loss of their virulence.

The term “inactivated vaccine” as used herein refers to a vaccine comprising microorganisms or virus particles that have been grown under controlled conditions and then killed or inactivated, typically by chemical treatment; e.g. treatment with formaldehyde, or heat treatment.

The terms “virus-like particle” and “VLP”, as used herein, refer to non-infectious particles resembling viruses that do not contain any viral genetic material. VLPs are the result of the expression of viral structural proteins, such as capsid proteins, and their self-assembly.

In the context of the present invention, the term “fractional vaccine” refers to a vaccine that comprises only part of a bacterium, virus or other microorganism. A “fractional vaccine” may either be protein-based or polysaccharide-based and may comprise a subunit, a toxoid (inactivated bacterial toxin), an unconjugated/pure polysaccharide and/or a conjugated polysaccharide, such as an unconjugated/pure or conjugated bacterial cell wall polysaccharide.

A “subunit vaccine” as used herein refers to a vaccine that comprises an isolated or purified vaccine antigen of a bacterium or virus or other microorganism, or a combination of several isolated and/or purified antigens, such as viral or bacterial polypeptides or nucleic acids encoding such polypeptides, capable of eliciting an immune response.

In the present context, the term “protein-based vaccine” refers to a vaccine comprising an isolated, purified or recombinant proteinaceous vaccine antigen, such as a proteinaceous antigen from a bacterium, virus or other microorganism.

In the context of the present invention, the term “polysaccharide-based vaccine” refers to a vaccine comprising an isolated or purified polysaccharide vaccine antigen, such as a polysaccharide derived from the capsule of a bacterium/a bacterial capsular polysaccharide.

The term “conjugate vaccine” refers broadly to a vaccine comprising two antigens conjugated to each other; typically a “weak” vaccine antigen is conjugated to a stronger antigen. Examples of conjugate vaccines include vaccines comprising a polysaccharide, such as a bacterial capsular polysaccharide conjugated to a protein to enhance immunogenicity.

The term “recombinant vaccine” refers to a vaccine comprising a vaccine antigen produced through recombinant DNA technology. Generally, this involves inserting a nucleotide sequence encoding the vaccine antigen into bacterial, fungal or mammalian cells, expressing the antigen in these cells and then purifying it from a culture of the bacterial, fungal or mammalian cells.

In the present context, the term “nucleic acid-based vaccine” refers broadly to a composition comprising a nucleic acid molecule encoding a vaccine antigen. The nucleic acid molecule may be a deoxyribonucleic acid (DNA); e.g. in the form of a plasmid, or a ribonucleic acid (RNA); e.g. in the form of mRNA. The may also be a nanoparticle-based nucleotide vaccine, e.g. a lipid-based, peptide-based, polysaccharide-based and/or inorganic nanoparticle formulation.

The term “viral vector-based vaccine” refers to a composition comprising a live replicating virus that has been subject to genetic engineering to carry a nucleic acid sequence encoding a vaccine antigen. The virus may e.g. be a retrovirus, a lentivirus, a vaccinia virus, an adenovirus, an adeno-associated virus, a cytomegalovirus, or a sendai virus.

In the context of the present invention, the terms “pediatric vaccine” and “childhood vaccine” are used interchangeably to refer to a vaccine intended for, recommended for and/or given to children to prevent or reduce the risk of infections and/or the development of infectious diseases.

Recommendations for routine vaccination/immunization are provided by the world Health Organization (WHO): While some routine vaccinations/immunizations are recommended for all children, there are also separate recommendations e.g. for children residing in certain geographic regions and for children in particular high-risk populations. Recommendations on routine vaccination may be found in position papers published by the WHO, concerning the following infectious diseases/agents and vaccines:
Tuberculosis/BCG vaccination: Weekly Epid. Record (2018, 93:73-96)
Hepatitis B: Weekly Epid. Record (2017, 92:369-392)
Polio/OPV and IPV: Weekly Epid. Record (2016, 9: 145-168)
Diphtheria: Weekly Epid. Record (2017, 92:417-436)
Tetanus: Weekly Epid. Record (2017, 92: 53-76)
Pertussis: Weekly Epid. Record (2015, 90: 433-460)
Rotavirus: Weekly Epid. Record (2013, 88: 49-64)
Measles: Weekly Epid. Record (2017, 92:205-228)
Pneumococcal: Weekly Epid. Record (2019, 94: 85-104)
Rubella: Weekly Epid. Record (2011, 86: 301-316)
Japanese Encephalitis: Weekly Epid. Record (2015, 90: 69-88)
Human papillomavirus (HPV): Weekly Epid. Record (2017, 92:241-268)
Yellow Fever: Weekly Epid. Record (2013, 88: 269-284)
Tick-Borne Encephalitis (TBE): Weekly Epid. Record (2011, 86: 241-256)
Typhoid: Weekly Epid. Record (2018, 93: 153-172)
Cholera: Weekly Epid. Record (2017, 92:477-500)
Hepatitis A: Weekly Epid. Record (2012, 87: 261-276)
Meningococcal reference: Weekly Epid. Record (2011, 86: 521-540); update for MenA conjugate:
Weekly Epid Record (2015, 90: 57-68)
Rabies: Weekly Epid. Record (2018, 93: 201-220)
Mumps: Weekly Epid. Record (2007, 82: 49-60)
Seasonal influenza (inactivated vaccine): Weekly Epid. Record (2012, 87: 461-476)
Dengue: Weekly Epid. Record (2018, 93, 457-76)
Varicella: Weekly Epid. Record (2014, 89: 265-288)

A “vaccination programme” as used herein refers to a series of vaccinations, including the timing of all doses, which may be either recommended or compulsory, depending on the country of residence.

A “booster vaccine” as used herein refers to a vaccine administered as a second or later vaccine dose, given after the primary dose(s) to increase the immune response to the vaccine antigen(s) in the primary vaccine dose(s). The vaccine given as the booster vaccine may or may not be the same as the primary vaccine.

“Cancer vaccine” as used herein, refers to a composition that induces a specific immunoresponse against a tumor, a tumor-associated antigen or a tumor-specific antigen. The term “tumor-associated antigen” refers to an antigen, which is present in greater amounts in or on tumor cells in a subject, as compared to the amounts present in or on non-tumor cells or cells in healthy tissue in said subjects. In terms of structure, a tumor-associated antigen is generally not qualitatively different from antigens found in or on normal cells, but they are present in significantly greater amounts.

The term “tumor-specific antigen” refers to an antigen, which is specifically expressed or produced in cells within a tumor and that is specifically expressed or upregulated in cells of the respective tumor, as compared to non-cancerous cells of the same origin. A tumor antigen, or epitopes derived therefrom, can be recognized by the immune system to induce an immune response. The tumor-specific antigen may be from all protein classes, e.g., enzymes, receptors, transcription factors, etc.

A “neoantigen” refers to an antigen that comprises a neoepitope; i.e. an antigenic epitope generated via random somatic mutations occurring in tumor cells. Neoepitopes are usually derived from specific tumor antigens or unique antigens that are specific for an individual cell or lineage of cells, and a neoepitope is thus specific to the cell or the lineage of tumor cells it is derived from.

A “variant” of a peptide or polypeptide of interest, as used herein, refers to an amino acid sequence that is altered by one or more amino acids, and hence deviates from a reference amino acid sequence. The variant may have “conservative” changes, wherein a substituted amino acid has similar structural or chemical properties, (e.g., replacement of leucine with isoleucine). More rarely, a variant may have “nonconservative” changes (e.g., replacement of a glycine with a tryptophan). Similar minor variations may also include amino acid deletions or insertions, or both. A variant peptide or polypeptide may have the same activity or function as the reference peptide or polypeptide, or may have greater or less activity or function than a reference peptide or polypeptide, but must at least retain partial activity or function of the reference peptide or polypeptide.

The term “adjuvant” as used herein refers to a substance or a mixture of substances that causes antigen-independent stimulation of the immune system or that is capable of potentiating the immunogenicity of an antigen. In the context of the present invention, an adjuvant may be used to enhance an immune response to a vaccine antigen.

In the context of the present invention the term “Th1-type immune response” refers to the activation of a subset of helper T cells, which is characterized by its ability to predominantly secrete inflammatory cytokines, typically interferon γ (IFNγ), tumor necrosis factor α (TNFα) and interleukin-2 (IL-2), upon activation. Th1-type immune responses predominantly result in an amplification of a cellular immune response against pathogens or cancerous cells.

The term “Th2-type immune response” as used herein refers to the activation of a subset of helper T cells, which is characterized by its ability to predominantly secrete cytokines such as IL-4, IL-5, and IL-13, which are associated with activation of eosinophils and promotion of allergic disease, and the anti-inflammatory cytokine IL-10. Th2-type immune responses predominantly facilitate the development of humoral immune responses, such as antibody responses.

As used herein, “adoptive T cell therapy” involves the isolation of a tumor-specific or tumor-non-specific T-cell and ex vivo expansion of a plurality of such tumor specific or tumor-nonspecific T cells to achieve a greater number of such T cells in a subject.

T cells may be collected in accordance with known techniques either from a tumor, such as a tumor of the subject that is to receive treatment according to the invention (tumor-infiltrating lymphocytes, TILs), or from peripheral blood (peripheral blood lymphocytes), such as from peripheral blood drawn from the subject that is to receive treatment according to the invention.

The T cells may be any appropriate type of T cell, including but not limited to cytotoxic T cells (CTLs; T lymphocytes that expresses CD8 on the surface (i.e. CD8+ T cells), CD4+ T cells, chimeric antigen receptor T cells (CAR T cells) and T-cell Receptor Gene-Transduced (TCRtg) Lymphocytes.

The T cells may be isolated from a human subject, but may in principle be isolated from any mammalian, preferably primate, species. The T cells may be allogenic (i.e. isolated from the same species but from a different donor) as the recipient subject; alternatively, the T cells may be autologous (i.e. the donor and the recipient are the same).

The plurality of T cells may comprise one single type of T-cell or may be a mixture of cell types. The number of cells needed will depend upon the ultimate use for which the T cell population is intended as will the type of cells included therein. For example, if cells that are specific for a particular antigen are desired, then the population may contain more than 70%, such as more than 80%, more than 85%, more than 90% or such as more than 95% of such cells.

The plurality of T cells may be enriched for the desired type of T cells by known techniques such as affinity binding to antibodies. After enrichment steps, in vitro expansion of the desired T cells can be carried out in accordance with known techniques (including but not limited to those described in U.S. Pat. No. 6,040,177), or variations thereof that will be apparent to those skilled in the art. For example, the desired T cell population or subpopulation may be expanded by adding an initial T lymphocyte population to a culture medium in vitro, and then adding to the culture medium feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). The order of addition of the T cells and feeder cells to the culture media may be reversed if desired. The culture can typically be incubated under conditions of temperature and the like that are suitable for the growth of T lymphocytes. For the growth of human T lymphocytes, for example, the temperature will generally be at least about 25 degrees Celsius, preferably at least about 30 degrees, more preferably about 37 degrees.

CAR T cells may be produced by techniques known in the art: In brief, T-cells are collected from a subject. Next, the T cells are transduced to express chimeric antigen receptors (CARs) comprising an antibody-binding domain fused to T cell signaling domain, using either a viral or a non-viral vector. After expansion the genetically engineered T cells are administered to the subject to be treated by infusion.

“Treatment cycle” is herein defined as a period of treatment followed by a period of rest (no treatment) that is repeated on a regular schedule. For example, a treatment cycle may comprise treatment given for one week followed by three weeks of rest. Also, a treatment cycle may comprise administration of a single dose of a medicament, followed by a period of rest, such as a period of one, two or three weeks of rest before another dose is administered. Multiple small doses in a small time window; e.g. within 2-24 hours, such as 2-12 hours or on the same day, may be considered equal to a larger single dose, especially if there is no clearance or no substantial clearance of the product between the doses.

The term “treatment” refers to the administration of an effective amount of one or more therapeutically active products; e.g. immunogenic compositions and binding agents (as defined in the present application) with the purpose of easing, ameliorating, arresting or eradicating symptoms or disease states.

The terms “therapeutically effective amount” or “effective amount,” as used interchangeably herein, can refer to the amount of a compound, such as an active ingredient in a pharmaceutical composition, that when administered alone or in combination with other compounds, such as other active ingredients, can be sufficient to prevent development of, or alleviate to some extent, one or more of the symptoms of the disorder, disease, or condition being treated. The term “therapeutically effective amount” can also refer to the amount of a compound that is sufficient to elicit the biological or medical response of a cell, tissue, system, animal, or human that is being sought by a researcher, veterinarian, medical doctor, or clinician.

The term “human CD3” as used herein, refers to the human Cluster of Differentiation 3 protein which is part of the T-cell co-receptor protein complex and is composed of four distinct chains. CD3 is also found in other species, and thus, the term “CD3” may be used herein and is not limited to human CD3 unless contradicted by context. In mammals, the complex contains a CD3γ (gamma) chain (e.g. human CD3γ chain Swissprot P09693, or cynomolgus monkey CD3γ Swissprot Q95LI7), a CD3δ (delta) chain (e.g. human CD3δ Swissprot P04234, or cynomolgus monkey CD3δ Swissprot Q95LI8), two CD3ε (epsilon) chains (e.g. human CD3ε Swissprot P07766; cynomolgus CD3ε Swissprot Q95LI5 or rhesus CD3ε (Swissprot G7NCB9)), and a CD3ζ-chain (zeta) chain (e.g. human CD3ζ Swissprot P20963, cynomolgus monkey CD3ζ Swissprot Q09TK0). These chains associate with a molecule known as the T-cell receptor (TCR) and generate an activation signal in T cells. The TCR and CD3 molecules together comprise the TCR complex.

SEQ ID NO: 1 shows the amino acid sequence of mature human CD3ε (epsilon), The term “mature” as used herein, refers to a protein, which does not comprise any signal or leader sequence.

It is well-known that signal peptide sequence homology, length, and the cleavage site position, varies significantly between different proteins. Signal peptides may be determined by different methods, for instance according to the SignalP application (available on http://www.cbs.dtu.dk/services/SignalP/).

In a particular embodiment, the binding agent of the present invention binds the epsilon chain of CD3, such as the epsilon chain of human CD3 (SEQ ID NO: 1). In yet another particular embodiment, the humanized or chimeric antibody binds an epitope within amino acids 1-27 of the N-terminal part of human CD3ε (epsilon) (SEQ ID NO: 1). In such a particular embodiment, the antibody may even further cross-react with other non-human primate species, such as cynomolgus monkeys (cynomolgus CD3 epsilon) and/or rhesus monkeys (rhesus CD3 epsilon).

The present invention is based on experimental data confirming that treatment of a tumor or cancer with a binding agent that binds to CD3 and a tumor target does not require the presence of tumor-specific T cells. Hence, the mere presence of T cells at the tumor sites appears to be sufficient for the binding agent to provide an effect on tumor growth, and vaccine-activated T cells appear to move into the tumor.

The data further show that a vaccine capable of boosting an existing T cell response in a subject, as for instance a childhood vaccine booster, may be used in combination with a binding agent that binds to CD3 and to a target antigen on tumor cells, thereby increasing the antitumor activity of the binding.

Hence, in a first aspect the present invention provides a method for preventing or reducing growth of a tumor, or for treatment of cancer in a subject in need thereof, comprising providing to the subject
i) a binding agent comprising an antigen-binding region that binds to CD3, such as human CD3, and an antigen-binding region that binds to a target antigen on cells of said tumor or cancer; and agent.
ii) an immunogenic composition comprising at least one vaccine antigen.

The vaccine antigen may in particular be a non-tumor specific vaccine antigen.

The immunogenic composition may be a vaccine, such as a prophylactic vaccine or a therapeutic vaccine.

It is currently preferred that the immunogenic composition is a vaccine against an infectious disease; e.g. a viral or bacterial infection as such vaccines generally are readily available and at low cost.

The vaccine may be against an infectious disease or infection, which is selected from the group consisting of cholera (V. cholerae; e.g. WC/rBS, Diphtheria (Corynebacterium diphtheriae), Haemophilus influenzae type b (Hib), hepatitis A, hepatitis B, human papillomavirus, influenza, coronavirus, encephalitis, measles, Lymphocytic choriomeningitis (LCM) (lymphocytic choriomeningitis mammarenavirus (LCMV)), coronavirus, encephalitis, measles, Lymphocytic choriomeningitis (LCM) (lymphocytic choriomeningitis mammarenavirus (LCMV)), meningococcal disease (Neisseria meningitidis), mumps, pertussis/whooping cough (Bordetella pertussis), pneumococcal disease/infections (Streptococcus pneumoniae), poliomyelitis (polio), rabies, rotavirus infection, rubella/German measles, tetanus (Clostridium tetani), smallpox, typhoid fever (Salmonella typhi), varicella/chickenpox, yellow fever, tuberculosis, plaque (Yersinia pestis), bat lyssavirus, Japanese encephalitis, Q fever (Coxiella burnetiid), varicella-zoster (chickenpox), anthrax (Bacillus anthracis).

The infection may in particular be a coronavirus infection, such as severe acute respiratory syndrome coronavirus 2 (SARS-CoV-2) infection.

The infectious disease may be Coronavirus disease 2019 (COVID-19).

The infection may be influenza infection.

The infectious disease may be influenza.

The infection may be lymphocytic choriomeningitis mammarenavirus (LCMV) infection.

The infectious disease may be lymphocytic choriomeningitis (LCM).

The immunogenic composition may be selected from the group consisting of
A live vaccine, such as a live attenuated vaccine,
an inactivated vaccine,
a split vaccine, such as a split virus vaccine,
a vaccine based on virus-like particles,
a fractional vaccine, such as a subunit vaccine, a protein-based vaccine, a peptide-based
vaccine, a polysaccharide-based vaccine, or a conjugated vaccine,
a recombinant vaccine; and
a nucleic acid based vaccine, such as a viral vector-based or mRNA-based vaccine.

In particular, the immunogenic composition may be a live vaccine, e.g. a live attenuated vaccine; such as
a live attenuated viral vaccine; e.g. live attenuated measles vaccine, live attenuated mumps vaccine, live attenuated rubella vaccine, live attenuated influenza vaccine, live attenuated chicken pox/varicella-zoster vaccine, live attenuated vaccinia (smallpox) vaccine, live attenuated oral polio vaccine (OPV)(Sabin), live attenuated rotavirus vaccine or live attenuated yellow fever vaccine;
or such as
a live attenuated bacterial vaccine; e.g. BCG vaccine, a live attenuated vaccine against Salmonella typhi (Ty21) (oral typhoid vaccine or epidemic typhus vaccine), a live attenuated cholera vaccine.

Alternatively, the immunogenic composition may be an inactivated vaccine; such as
an inactivated virus vaccine; e.g. inactivated poliovirus vaccine (IPV) (Salk vaccine), inactivated influenza vaccine, inactivated rabies vaccine, inactivated Japanese encephalitis vaccine or an inactivated hepatitis A vaccine;
or such as
an inactivated bacterial vaccine, e.g. inactivated typhoid vaccine, inactivated cholera vaccine, inactivated plague vaccine, inactivated Q fever vaccine, inactivated anthrax vaccine or inactivated pertussis vaccine.

The fractional vaccine may be a protein-based vaccine selected from the group consisting of
a toxoid vaccine, e.g. a vaccine comprising a tetanus toxoid or a diphtheria toxoid;
a subunit vaccine; and
a subvirion vaccine.

The fractional vaccine may be a polysaccharide-based vaccine; e.g. polysaccharide-based meningococcal disease (Neisseria meningitidis (A, C, Y.W135)) vaccine, a polysaccharide-based Salmonella typhi vaccine or polysaccharide-based pneumococcal disease (Streptococcus pneumoniae) vaccine.

The fractional vaccine may be a conjugated vaccine comprising a polysaccharide linked to a polypeptide; e.g. conjugated Haemophilia influenza type b (Hib) vaccine, conjugated Streptococcus pneumoniae vaccine, conjugated Neisseria meningitidis vaccine.

The immunogenic composition may be a recombinant vaccine.

The World Health Organization (WHO) provides a list of prequalified vaccines, which is available at: https://extranet.who.int/gavi/PQ_Web/. The WHO prequalification of vaccines is a comprehensive assessment that takes place through a standardized procedure aimed at determining whether the product meets requirements for safety and efficacy in immunization programmes. Hence, the WO prequalified vaccines are safe, effective and available to be used in according to the invention in combination with the binding agent as defined herein.

TABLE 3
WHO list of prequalified vaccines

| Vaccine type | Name | Manufacturer |
|---|---|---|
| BCG | BCG Freeze Dried Glutamate | Japan BCG Laboratory |
| | vaccine | |
| | BCG Freezed Dried Glutamate | Japan BCG Laboratory |
| | vaccine | |
| | BCG Vaccine | BB- NCIPD Ltd. |
| | BCG Vaccine | Serum Institute of India Pvt. |
| | | Ltd. |
| | BCG Vaccine SSI | AJ Vaccines A/S |
| Cholera: inactivated oral | Shanchol | Sanofi Healthcare India Private |
| | | Limited |
| | Dukoral | Valneva Sweden AB |
| | Euvichol | EuBiologics Co., Ltd. |
| | Euvichol-Plus | EuBiologics Co., Ltd. |
| Dengue Tetravalent (live, | Dengvaxia | Sanofi Pasteur SA |
| attenuated) | | |
| Diphtheria-Tetanus | Diphtheria and Tetanus Vaccine | Serum Institute of India Pvt. |
| | Adsorbed (Paediatric) | Ltd. |
| | Adsorbed DT Vaccine | PT Bio Farma (Persero) |
| | Diftet | BB- NCIPD Ltd. (Bul Bio - |
| | | National Center of Infectious |
| | | and Parasitic Diseases Ltd.) |
| Diphtheria-Tetanus (reduced | Tetadif | BB- NCIPD Ltd. |
| antigen content) | Diphtheria and Tetanus Vaccine | Serum Institute of India Pvt. |
| | Adsorbed for Adults and | Ltd. |
| | Adolescents | |
| | IMOVAX dT adult | Sanofi Pasteur SA |
| Diphtheria-Tetanus-Pertussis | Adacel | Diphtheria-Tetanus-Pertussis |
| (acellular) | | (acellular) |
| | Boostrix | GlaxoSmithKline Biologicals SA |
| Diphtheria-Tetanus-Pertussis | Hexaxim | Sanofi Pasteur SA |
| (acellular)-Hepatitis B- | | |
| Haemophilus influenzae type | | |
| b-Polio (Inactivated) | | |
| Diphtheria-Tetanus-Pertussis | Diphtheria-Tetanus-Pertussis | Serum Institute of India Pvt. |
| (whole cell) | Vaccine Adsorbed | Ltd . |
| | DTP Vaccine | PT Bio Farma (Persero) |
| | TRIPVAC | Biological E. Limited |
| Diphtheria-Tetanus-Pertussis | Diphtheria, Tetanus, Pertussis | Serum Institute of India Pvt. |
| (whole cell)-Haemophilus | and Haemophilus influenzae | Ltd . |
| influenzae type b | type b Conjugate Vaccine | |
| Diphtheria-Tetanus-Pertussis | Diphtheria ,Tetanus, Pertussis | Serum Institute of India Pvt. |
| (whole cell)-Hepatitis B | and Hepatitis B Vaccine | Ltd. |
| | Adsorbed | |
| Diphtheria-Tetanus-Pertussis | Diphtheria, Tetanus, Pertussis, | Serum Institute of India Pvt. |
| (whole cell)-Hepatitis B- | Hepatitis B and Haemophilus | Ltd. |
| Haemophilus influenzae type b | influenzae type b Conjugate | |
| | Vaccine | |
| | Diphtheria, Tetanus, Pertussis, | Serum Institute of India Pvt. |
| | Hepatitis B and Haemophilus | Ltd. (India) |
| | influenzae type b Conjugate | |
| | Vaccine Adsorbed | |
| | Pentabio | PT Bio Farma (Persero) |
| | ComBE Five (Liquid) | Biological E. Limited |
| | ComBE Five (Reconstituted). | Biological E. Limited |
| | Eupenta | LG Chem Ltd |
| | Shan-5 | Sanofi Healthcare India Private |
| | | Limited |
| | Easyfive-TT | Panacea Biotec Ltd. |
| Ebola Zaire (rVSVAG-ZEBOV- | ERVEBO | Merck Vaccines |
| GP, live attenuated) | | |
| Haemophilus influenzae type b | Act-HIB | Sanofi Pasteur SA |
| | Haemophilus influenzae type b | Serum Institute of India Pvt. |
| | Conjugate Vaccine | Ltd. |
| | Quimi-Hib | Centro de Ingenieria Genetica y |
| | | Biotecnologia |
| Hepatitis A (Human Diploid | HEALIVE | Sinovac Biotech Co. Ltd |
| Cell), Inactivated (Adult) | Havrix 1440 Adult | GlaxoSmithKline Biologicals SA |
| Hepatitis A (Human Diploid | HEALIVE | Sinovac Biotech Co. Ltd |
| Cell), Inactivated (Paediatric) | Havrix 720 Junior | GlaxoSmithKline Biologicals SA |
| Hepatitis B | Heberbiovac HB | Centro de Ingenieria Genetica y |
| | | Biotecnologia |
| | Engerix | GlaxoSmithKline Biologicals SA |
| | Hepatitis B Vaccine (rDNA) | Serum Institute of India Pvt. |
| | (Adult) | Ltd. |
| | Hepatitis B Vaccine (rDNA) | Serum Institute of India Pvt. |
| | (Paediatric) | Ltd. |
| | Euvax B | LG Chem Ltd |
| Hepatitis B (Paediatric) | Euvax B | LG Chem Ltd |
| Human Papillomavirus | Cervarix | GlaxoSmithKline Biologicals SA |
| (Bivalent) | | |
| Human Papillomavirus | Gardasil 9 | Merck Vaccines |
| (Ninevalent) | | |
| Human Papillomavirus | Gardasil | Merck Vaccines |
| (Quadrivalent) | | |
| Influenza, pandemic H1N1 | Influenza A (H1N1) 2009 | MedImmune |
| | monovalent vaccine | |
| | NASOVAC Influenza Vaccine, | Serum Institute of India Pvt. |
| | Live Attenuated (Human) | Ltd. |
| | Freeze-Dried | |
| | Panenza | Influenza, pandemic H1N1 |
| | Green Flu-S | Green Cross Corporation |
| | Panvax | Seqirus Limited |
| | Celtura | Seqirus GmbH |
| | Fluvirin-H1N1 | Seqirus Vaccines Limited |
| | Focetria | Seqirus Vaccines Limited |
| | Influenza A (H1N1) 2009 | Sanofi Pasteur-USA |
| | monovalent vaccine | |
| Influenza, seasonal | Fluzone Quadrivalent | Sanofi Pasteur-USA |
| (Quadrivalent) | SKYCellflu Quadrivalent inj. | SK Bioscience Co., Ltd. |
| | SKYCellflu Quadrivalent Multi | SK Bioscience Co., Ltd. |
| | inj. | |
| | Afluria ® Quadrivalent | Seqirus Limited |
| | GCFLU Quadrivalent inj. | Green Cross Corporation |
| | GCFLU Quadrivalent Multi inj. | Green Cross Corporation |
| Influenza, seasonal (Trivalent) | GC FLU inj. | Green Cross Corporation |
| | GC FLU Multi inj. | Green Cross Corporation |
| | SKYCellflu ® inj. | SK Bioscience Co., Ltd. |
| | SKYCellflu ® Multi inj. | SK Bioscience Co., Ltd. |
| | Afluria ® | Seqirus Limited |
| | Influenza Vaccine (Split virion, | Hualan Biological Bacterin Co., |
| | inactivated) | Ltd |
| | Fluzone | Sanofi Pasteur-USA |
| | Serinflu | Abbott Biologicals BV |
| | IL-YANG FLU Vaccine INJ. | IL-YANG PHARMACEUTICAL |
| | | CO., LTD. |
| | Fluvirin | Seqirus Vaccines Limited |
| | AGRIFLU | Seqirus Vaccines Limited |
| | Vaxigrip | Sanofi Pasteur SA |
| | Nasovac-S Influenza Vaccine, | Serum Institute of India Pvt. |
| | Live, Attenuated (Human) | Ltd. |
| Japanese Encephalitis Vaccine | JEEV ® (3 μg) | Biological E. Limited |
| (Inactivated) (3 μg Pediatric) | | |
| Japanese Encephalitis Vaccine | JEEV ® (6 μg) | Biological E. Limited |
| (Inactivated) 6 μg | | |
| Japanese Encephalitis Vaccine | Japanese Encephalitis Vaccine | Chengdu Institute of Biological |
| (live, attenuated) | Live (SA14-14-2) | Products Co.,Ltd |
| | IMOJEV MD | GPO-MBP Co., Ltd. |
| Measles | Measles vaccine | PT Bio Farma (Persero) |
| | Measles Vaccine, Live, | Serum Institute of India Pvt. |
| | Attenuated | Ltd. |
| Measles and Rubella | Measles and Rubella Vaccine, | Serum Institute of India Pvt. |
| | Live, Attenuated | Ltd. |
| | Measles And Rubella Vaccine | Biological E. Limited |
| | (Live) (Attenuated, Freeze | |
| | Dried) | |
| Measles, Mumps and Rubella | rHA M-M-R II | Merck Vaccines |
| | Measles, Mumps and Rubella | Serum Institute of India Pvt. |
| | Vaccine, Live, Attenuated | Ltd. |
| | Priorix | GlaxoSmithKline Biologicals SA |
| Meningococcal A Conjugate | Meningococcal A Conjugate | Serum Institute of India Pvt. |
| 10 μg | MenAfriVac | Ltd. |
| Meningococcal A Conjugate | Meningococcal A Conjugate 5 | Serum Institute of India Pvt. |
| 5 μg | micrograms MenAfriVac 5 μg | Ltd. |
| Meningococcal ACYW-135 | Nimenrix | Pfizer |
| (conjugate vaccine) | Menactra | Sanofi Pasteur-USA |
| | Menveo | GlaxoSmithKline Vaccines S.r.l. |
| Pneumococcal (conjugate) | Synflorix | GlaxoSmithKline Biologicals SA |
| Polio Vaccine - Inactivated (IPV) | IPV Vaccine SSI | AJ Vaccines A/S |
| | Picovax | AJ Vaccines A/S |
| | Poliomyelitis Vaccine | Serum Institute of India Pvt. |
| | (Inactivated) | Ltd. |
| | IMOVAX POLIO | Sanofi Pasteur SA |
| | Poliomyelitis vaccine | Bilthoven Biologicals B.V. |
| | Poliomyelitis vaccine | Bilthoven Biologicals B.V. |
| | multidose, suspension for | |
| | injection 2.5 mL | |
| | ShanIPV | Sanofi Healthcare India Private |
| | | Limited |
| | Poliorix | GlaxoSmithKline Biologicals SA |
| Polio Vaccine - Oral (OPV) | Polio Sabin One and Three | GlaxoSmithKline Biologicals SA |
| Bivalent Types 1 and 3 | Bharat Biotech International | BIOPOLIO B1/3 |
| | Limited | |
| | Bivalent OPV Type 1 and 3 | Panacea Biotec Ltd. |
| | Poliomyelitis Vaccine, Live | |
| | (Oral) | |
| | Poliomyelitis Vaccine (live, oral | Beijing Institute of Biological |
| | attenuated, human Diploid | Products Co., Ltd. |
| | Cell), type 1 and 3 | |
| | Poliomyelitis Vaccine (Oral), | Serum Institute of India Pvt. |
| | Bivalent types 1 and 3 | Ltd. |
| | Bivalent type 1&3 Oral | Haffkine Bio Pharmaceutical |
| | Poliomyelitis vaccine, IF | Corporation Ltd |
| | (bOPV1&3) | |
| | Bivalent Oral Poliomyelitis | PT Bio Farma (Persero) |
| | Vaccine Type 1&3 (bOPV 1&3) | |
| Polio Vaccine - Oral (OPV) | Monovalent Oral Poliomyelitis | PT Bio Farma (Persero) |
| Monovalent Type 1 | Vaccine Type 1 (mOPV1) | |
| | Monovalent type 1 Oral | Haffkine Bio Pharmaceutical |
| | Poliomyelitis vaccine, | Corporation Ltd |
| | (mOPV1) | |
| | ORAL MONOVALENT TYPE 1 | Sanofi Pasteur SA |
| | POLIOMYELITIS VACCINE | |
| | Polio Sabin Mono T1 | GlaxoSmithKline Biologicals SA |
| | Polio Sabin Mono Two (oral) | GlaxoSmithKline Biologicals SA |
| | ORAL MONOVALENT TYPE 2 | Sanofi Pasteur SA (France) |
| | POLIOMYELITIS VACCINE | |
| | (mOPV2) | |
| | Monovalent Oral Poliomyelitis | PT Bio Farma (Persero) |
| | Vaccine Type 2 | |
| | ORAL MONOVALENT TYPE 3 | Sanofi Pasteur SA |
| | POLIOMYELITIS VACCINE | |
| | Polio Sabin Mono Three (oral) | GlaxoSmithKline Biologicals SA |
| Polio Vaccine - Oral (OPV) | BIOPOLIO | Bharat Biotech International |
| Trivalent | | Limited |
| | OPVERO | Sanofi Pasteur SA |
| | Oral polio | PT Bio Farma (Persero) |
| | Polioviral vaccine | Haffkine Bio Pharmaceutical |
| | | Corporation Ltd |
| Rabies | VERORAB | Sanofi Pasteur SA |
| | Rabies Vaccine Inactivated | Serum Institute of India Pvt. |
| | (Freeze Dried)(RABIVAX-S) | Ltd. |
| | VaxiRab N | Cadila Health Care Ltd |
| Rotavirus | Rotarix | GlaxoSmithKline Biologicals SA |
| | Rotateq | Merck Vaccines |
| Rotavirus (live, attenuated) | ROTASIIL | Serum Institute of India Pvt. |
| | | Ltd. |
| | ROTASIIL ®Thermo | Serum Institute of India Pvt. |
| | | Ltd . |
| | Rotavac | Bharat Biotech International |
| | | Limited |
| Rubella | Rubella Vaccine, Live, | Serum Institute of India Pvt. |
| | Attenuated | Ltd. |
| Tetanus Toxoid | Tetanus Toxoid Vaccine | Serum Institute of India Pvt. |
| | Adsorbed | Ltd. |
| | TETAVAX | Sanofi Pasteur SA |
| | Bett | Biological E. Limited |
| | ShanTT | Sanofi Healthcare India Private |
| | | Limited |
| | Tetatox | BB- NCIPD Ltd. (Bul Bio- |
| | | National Center of Infectious |
| | | and Parasitic Diseases Ltd.) |
| | TT vaccine | PT Bio Farma (Persero) |
| Typhoid (Conjugate) | Typbar-TCV | Bharat Biotech International |
| | | Limited |
| Typhoid (Polysaccharide) | Typhim-Vi | Sanofi Pasteur SA |
| Varicella | Varivax | Merck Vaccines |
| | SKYVaricella Inj. | SK Bioscience Co., Ltd. |
| Yellow Fever | Stabilized Yellow Fever Vaccine | Institut Pasteur de Dakar |
| | STAMARIL | Sanofi Pasteur SA |
| | Yellow Fever | Bio-Manguinhos/Fiocruz |

Hence, the immunogenic composition used according to the invention may be selected from the group consisting of: BCG; Cholera: inactivated oral; Dengue Tetravalent (live, attenuated); Diphtheria-Tetanus; Diphtheria-Tetanus (reduced antigen content); Diphtheria-Tetanus-Pertussis (acellular) (DTaP/Tdap); Diphtheria, Tetanus, acellular Pertussis and Haemophilus influenzae type b (DTaP-Hib); Diphtheria-Tetanus-Pertussis (acellular)-Hepatitis B-Haemophilus influenzae type b-Polio (Inactivated); Diphtheria-Tetanus-Pertussis (whole cell) (DTP); Diphtheria-Tetanus-Pertussis (whole cell)-Haemophilus influenzae type b (DTP-Hib); Diphtheria-Tetanus-Pertussis (whole cell)-Hepatitis B; Diphtheria-Tetanus-Pertussis (whole cell)-Hepatitis B-Haemophilus influenzae type b; Ebola Zaire (rVSVΔG-ZEBOV-GP, live attenuated); Haemophilus influenzae type b (Hib); Hepatitis A (Human Diploid Cell), Inactivated (Adult); Hepatitis A (Human Diploid Cell), Inactivated (Paediatric); Hepatitis B, Hepatitis B (Paediatric); Human Papillomavirus (Bivalent); Human Papillomavirus (Ninevalent); Human Papillomavirus (Quadrivalent); Influenza, pandemic H1N1; Influenza, seasonal (Quadrivalent); Influenza, seasonal (Trivalent); Japanese Encephalitis Vaccine (Inactivated); Japanese Encephalitis Vaccine (live, attenuated); Measles; Measles and Rubella; Measles, Mumps and Rubella (MMR); Measles, Mumps, Rubella and Varicella (MMRV) Measles, Mumps and Rubella (MMR); Meningococcal A Conjugate 10 μg; Meningococcal A Conjugate 5 μg; Meningococcal ACYW-135 (conjugate vaccine); Pneumococcal (conjugate); Polio Vaccine—Inactivated (IPV); Polio Vaccine—Oral (OPV) Bivalent Types 1 and 3; Polio Vaccine—Oral (OPV) Monovalent Type 1; Polio Vaccine—Oral (OPV) Trivalent; Rabies; Rotavirus; Rotavirus (live, attenuated); Rubella; Tetanus Toxoid; Typhoid (Conjugate); Typhoid (Polysaccharide); Varicella; Yellow Fever, Bacille Calmette-Guerin (BCG).

The immunogenic composition may be a COVID-19 vaccine.

The COVID-19 vaccine may be selected from the group consisting of BNT162b2/COMIRNATY (Tozinameran) (Pfizer BioNTech), CVnCoV/CV07050101 (Zorecimeran) (CureVac), AZD1222 Vaxzevria (AstraZeneca), mRNA-1273 (Moderna), SARS-CoV-2 Vaccine (Vero Cell), Inactivated (InCoV) (Sinopharm/Beijing Institute of Biological Products Co., Ltd. (BIBP), CoV2 preS dTM-AS03 vaccine (Sanofi) and Covishield (ChAdOx1_nCoV-19) (Serum Institute of India Pvt. Ltd), Sputnik V (rAd26 and rAd5) (Acellena), Sputnik Light (rAd26) (Acellena), Ad26.COV2.S (JNJ78436735) (Janssen), CoronaVav (Sinovac), BBIBP-CorV (Beijing Institute of Biological Products; China National Pharmaceutical Group (Sinopharm)), EpiVacCorona (Federal Budgetary Research Institution State Research Center of Virology and Biotechnology), Convidicea (CanSino Biologics) and MVC-COV1901 (Medigen Vaccine Biologics Corp.; Dynavax).

The immunogenic composition may be an influenza vaccine.

The immunogenic composition may be an influenza vaccine of a type selected from the group consisting of Influenza Pandemic (H1N1), Influenza seasonal (Trivalent), and Influenza seasonal (Quadrivalent).

The infectious disease may be lymphocytic choriomeningitis (LCM) vaccine.

The immunogenic composition may be a pediatric vaccine or childhood vaccine.

The immunogenic composition may in particular be a childhood booster vaccine.

Alternatively, the immunogenic composition may be a cancer vaccine, such as a prophylactic or therapeutic cancer vaccine.

The vaccine antigen may be a tumor-associated antigen or a tumor-specific antigen.

The immunogenic composition may comprise a vaccine antigen expressed by said tumor or cancer.

The target antigen and the vaccine antigen may both be expressed by the said tumor or cancer.

According to such embodiments, the binding agent and the immunogenic composition are directed against or target the same tumor or cancer.

In the method according to the invention, the vaccine antigen and the target antigen may be the same, or the vaccine antigen may be a part, a subsequence or a variant of the target antigen.

The immunogenic composition may comprise a vaccine antigen selected from the group consisting of an antigen that is overexpressed by said tumor, such as Her2/neu, survivin or Muc-1; a cancer neoantigen, such as a p53 neoantigen; a cancer testis antigen, such as MAGE-A3, MAGE-A2, MAGE-A4, PRAME, CT83, SSX2 or NY-ESO-1; a differentiation antigen, such as Mart1, PSA or PAP and a viral-associated antigens, such as a HPV antigen.

The immunogenic composition may be a personalized cancer vaccine and the vaccine antigen may be a neoantigen, which is specific to the subject's tumor.

Preferably, the vaccine antigen comprises one or more T-cell epitopes.

Also, the immunogenic composition is preferably capable of eliciting a cytotoxic T-cell response in vivo and/or in vitro.

It is further preferred that the vaccine is capable of eliciting a T-cell response, which comprises T-cell activation and/or T-cell infiltration of said tumor or cancer.

Also, the immunogenic composition used in the method according to the invention may be capable of eliciting a T-cell response, which comprises T-cell infiltration of said tumor or cancer and activation of tumor-infiltrating T-cells.

The immunogenic composition used in the method according to the invention may be capable of eliciting infiltration and/or expansion of immune cells in tumors, such as infiltration and/or expansion natural killer (NK) cells and/or dendritic cells (DCs).

The skilled person will readily be able to determine the ability of an immunogenic composition to elicit T cell infiltration or T cell activation. As an example, T cell infiltration and/or T cell activation may be determined in mice carrying a tumor that expresses an antigen to which the 2^{nd} binding region of said binding agent binds, using a procedure in which the mice are subject to transfer of tumor specific T-cells and then injected subcutaneously with the immunogenic composition and subsequently with the binding agent.

By way of example T cell infiltration and/or activation may be determined in a procedure comprising the steps of:
i) Providing T cells which express an antigen to which the binding agent is able to bind, and constitutively express a 1^{st} oxidative enzyme capable of producing a 1^{st} bioluminescence and 2^{nd} oxidative enzyme capable of producing a 2^{nd} bioluminescence, wherein expression of the 2^{nd} oxidative is induced by T-cell activation, such as by Nuclear Factor of Activated T cells (NFAT),
ii) Injecting a mouse, such as Albino C57BL/6 mice, carrying a tumor that expresses an antigen to which the tumor-targeting binding region of said binding agent binds, with the T cell defined in i) by intravenous injection in the tail vein,
iii) Administering to the mouse two doses of the immunogenic composition by subcutaneous injection at the tail base, 1 and 8 days after injection of the T cells in ii), iv) Administering to the mouse two doses of said binding agent by intravenous injection or infusion, 10 and 14 days after injection of the T cells in ii),
v) Injecting the mouse with a substrate of each of said oxidative enzymes and measuring said 1^{st} and 2^{nd} bioluminescence.

Further, by way of example, T-cell infiltration and/or activation is determined using a procedure, which is essentially as set forth in Example 4 herein, or using the procedure set forth in Example 4.

In further embodiments, the method according to the invention comprises determining existing T-cell immunity in said subject. The existing T cell immunity may be from previous from previous vaccination, for instance with a vaccine as defined above. Alternatively, the existing T cell immunity may be a result of previous infection, including infection with any one of the pathogenic agents defined above, or as a result of an immune response to said tumor The existing T-cell immunity is determined by measurement of peripheral T-cell activation.

The method according to the invention may comprise determining the subject's previous participation in a vaccination program, such as a childhood vaccination program. This may be a convenient way of determining existing T-cell immunity that may be boosted as part of the method according to the invention, in particular in countries or regions where childhood vaccination programs have been well established and well documented for many years.

The method according to the invention may use an immunogenic composition, which is a vaccine against an infection or infectious disease, which the subject has previously had, and/or has developed a T-cell immune response against; such as any of the infections or infectious diseases defined above.

The immunogenic composition may be formulated with pharmaceutically acceptable carriers or diluents as well as any other known adjuvants and excipients in accordance with conventional techniques. The pharmaceutically acceptable carriers or diluents as well as any other known excipients should be suitable for use together with vaccine antigen and the chosen mode of administration. The immunogenic composition used according to the present invention may also include diluents, fillers, salts, buffers, detergents (e. g., a nonionic detergent, such as Tween-20 or Tween-80), stabilizers (e.g., sugars or protein-free amino acids), preservatives, tissue fixatives, solubilizers, and/or other materials suitable for inclusion in a pharmaceutical composition.

The method according to the invention may further comprise administration of an adjuvant.

Adjuvants are available, which are capable of eliciting both a Th1 and a Th2 response, as are adjuvants which have the ability of driving an immune response towards a Th1 response or towards a Th2 response. Hence the adjuvant used in the method according to the invention may be a Th1/Th2 adjuvant, a Th1 adjuvant or a Th2 adjuvant; preferably a Th1 adjuvant.

Likewise, the immunogenic composition, when administered together with the adjuvant may be capable of eliciting an NK cell response and/or a Th1/Th2-type immune response, a Th1-type immune response or a Th2-type immune response; preferably a Th1-type immune response.

The immunogenic composition used in the method according to the invention may be one that comprises an adjuvant. In such embodiments in particular it is contemplated that separate administration of an adjuvant may not provide any additional benefit.

According to these embodiments, the immunogenic composition comprising the adjuvant may be capable of eliciting a Th1/Th2-type immune response, a Th1-type immune response, or a Th2-type immune response; preferably a Th1-type immune response. In the context of the present invention a Th1-type immune response may be preferred since the response, in turn, generates cytotoxic T cells that have the ability to target and lyse antigen-expressing tumor cells.

For the purpose of the present invention, an adjuvant may be used that comprises an aluminum salt; e.g. alum (XAl(SO₄)₂·12H₂O; where X is a monovalent cation, such as K⁺ or NH₄⁺). Traditionally, adjuvants comprising aluminum salts have been commonly used in vaccines against infectious bacteria and viruses.

Alternatively the adjuvant may be an emulsion-based adjuvant, such as an oil-in-water emulsion.

In particular embodiments, the adjuvant is selected from the group of adjuvants that are commonly used in licensed vaccines (Del Giudice et al., Seminars in Immunology 39, 14-21, 2018). Hence, the adjuvant may be selected form the group consisting of
a) an adjuvant comprising squalene, polysorbate 80 and sorbitan trioleate; e.g. MF59,
b) an adjuvant comprising squalene, polysorbate 80 and α-tocopherol; e.g. AS03
c) an adjuvant comprising squalene, polyoxyethylene, cetostearyl ether, mannitol and sorbitan oleate; e.g. AF03.
d) An adjuvant comprising 3-O-desacyl-4′-monophosphoryl lipid A (MPL), Quillaja saponaria Molina, fraction 21 (QS-21) and liposome; e.g. AS01; and
e) An adjuvant comprising 3-O-desacyl-4′-monophosphoryl lipid A (MPL) and aluminum hydroxide; e.g. AS04.

Alternatively, the adjuvant may be a Toll-like receptor (TLR) agonist, such as a TLR7 agonist selected from the group consisting of Imiquimod (Aldara), resiquimod and gardiquimod.

The adjuvant may comprise
i) a Toll-like receptor 9 (TLR9) agonist, such as a TLR9 agonist selected from the group consisting of Neisseria meningitidis porin B (porB), a CpG Oligodeoxynucleotide (ODN) and tilsotolimod,
ii) a Toll-like receptor 4 (TLR4) agonist, such as a TLR4 agonist selected from the group consisting of monophosphoryl lipid A (MPL), glucopyranosyl lipid A (GLA) and neoseptin-3,
iii) a Toll-like receptor 5 (TLR5) agonist, such as a TLR5 agonist selected from the group consisting of mobilan, entolimod or recombinant flagellin FlicC; and/or
iv) a Toll-like receptor 3 (TLR3) agonist, such as a TLR3 agonist selected from the group consisting of Poly-IC and derivatives thereof.

The adjuvant may comprise nanoparticles, such as lipid nanoparticles (LPNs); e.g. adjuvant-incorporated lipid nanoparticles.

The immunogenic composition used in the method according to the invention may comprise a cytokine. Alternatively, a cytokine may be administered in combination with the immunogenic composition, or a cytokine may be included in or combined with an adjuvant, which is optionally administered to the subject in combination with the immunogenic composition, such as any of the adjuvants disclosed above.

Hence, the method according to the invention may comprise a step of administering the immunogenic composition in combination with a cytokine. The cytokine is preferably an interleukin-2 (IL2) receptor agonist, such as human IL2/aldesleukin, or human interleukin-15 or an analog of any of the two. The amino acid sequence of human interleukin-2 (mature sequence, excluding predicted signal peptide) is set forth in SEQ ID NO: 55. The amino acid sequence of human interleukin-15 (mature sequence, excluding predicted signal peptide and pro-peptide sequence) is set forth in SEQ ID NO: 56.

Further, modified or genetically engineered cytokines may be used according to the invention; e.g. half-life extended, immunocytokines, conditionally active, CD25-null binding cytokines. Particular examples include NKTR-214 (Bempegaldesleukin; PEGylated IL2) Nektar Therapeutics, Neo-2/15 (de novo designed IL-2/IL-15 mimick) Neoleukin, IL-2v (CD25-null binding) Roche.

In the method according to the invention, the binding agent may be administered to the subject by parenteral or systemic administration, such as by injection or infusion; e.g. by intravenous injection or infusion.

Preferably, the binding agent is provided to the subject in one or more treatment cycles.

The binding agent may be dosed once a week (1Q1W), once every second week (1Q2W), once every third week (1Q3W) or once every fourth week (1Q4W).

The immunogenic composition may be administered to the subject to achieve a topical effect and/or may be administered by topical administration, such as by injection into the tumor to be treated.

Alternatively, the immunogenic composition may be administered to achieve a systemic effect and/or it may be administered by systemic administration, such as by oral administration, subcutaneous injection, intramuscular injection, intradermal injection or by a transmucosal route.

The adjuvant may be administered to the subject to achieve a topical effect/by topical administration. For example, the adjuvant may be administered by injection into said tumor.

The adjuvant may be administered to achieve a systemic effect/by systemic administration, such as by oral administration, subcutaneous injection, intramuscular injection, intradermal injection or by a transmucosal route.

The cytokine may be administered to the subject by parenteral or systemic administration, such as by injection or infusion; e.g. by intravenous injection or infusion. It may also be administered to the subject in the form of a nucleic acid formulation (e.g. mRNA), for in situ production of said cytokine.

Prior to receiving treatment according to the invention, the subject may have received treatment with an immunogenic composition, for instance a vaccine, and possible also an adjuvant for a different purpose, for instance to treat an infection or an infectious disease. Also, the subject may have received treatment with a cytokine, for instance IL-2, prior to the treatment according to the invention. IL-2 has been approved for the treatment of metastatic renal cell carcinoma and metastatic melanoma and may have thus have been provided to the subject as cancer immunotherapy. However, it is to be understood that in the method according to the invention the immunogenic composition and optionally the adjuvant and/or the cytokine, such as the interleukin 2 receptor agonist, is/are preferably administered as part of a treatment regimen/the same treatment regimen provided to reduce growth of said tumor, and/or to treat said cancer.

In further embodiments of the invention, administration of the immunogenic composition and administration of the binding agent are combined with adoptive T-cell therapy. Hence, the method according to the invention may comprise administering a plurality of T-cells to the subject.

The immunogenic composition may be administered to said subject simultaneously with or on the same day as administration of the binding agent, simultaneously with or on the same day as administration of the first dose of the binding agent and/or as part of the first treatment cycle with the binding agent.

Administration of the immunogenic composition and administration of the binding agent, such as administration of the immunogenic composition and administration of the first dose of the binding agent and/or onset of the first treatment cycle with the binding agent, may separated by a time period of at the most 2 months, such as at the most 1 month, at the most 4 weeks, at the most 3 weeks, at the most 2 weeks, at the most 1 week, at the most 6 days, at the most 5 days, at the most 4 days, at the most 3, days or at the most 2 days.

Administration of the immunogenic composition and administration of the binding agent, such as administration of the immunogenic composition and administration of the first dose of the binding agent and/or onset of the first treatment cycle with the binding agent, may be separated by a time period of 2 days to 2 months, such as 2 days to 1 month, 2 days to 4 weeks, 2 days to 3 weeks, 2 days to 2 weeks, 2 days to 1 week, 3 days to 2 months, 3 days to 1 month, 3 days to 4 weeks, 3 days to 3 weeks, 3 days to 2 weeks, 3 days to 1 week, 4 days to 2 months, 4 days to 1 month, 4 days to 4 weeks, 4 days to 3 weeks, 4 days to 2 weeks, 4 days to 1 week, 5 days to 2 months, 5 days to 1 month, 5 days to 4 weeks, 5 days to 3 weeks, 5 days to 2 weeks, 5 days to 1 week, 6 days to 2 months, such as 6 days to 1 month, 6 days to 4 weeks, 6 days to 3 weeks, 6 days to 2 weeks, 1 week to 2 months, 1 week to 1 month, 1 to 4 weeks, 1 to 3 weeks, 1 to 2 weeks, 3 weeks to 2 months, 3 weeks to 1 month or 3 to 4 weeks.

The immunogenic composition may be administered to said subject prior to administration of the binding agent, prior to administration of a first dosage of the binding agent and/or prior to the first treatment cycle with the binding agent.

The immunogenic composition may be administered to the subject from 2 days to 2 months prior to administration of the binding agent, prior to administration of a first dosage of the binding agent and/or prior to the first treatment cycle with the binding agent; such as from 2 days to 1 month, from 2 days to 4 weeks, from 2 days to 3 weeks, from 2 days to 2 weeks, from 2 days to 1 week, from 3 days to 2 months, from 3 days to 1 month, from 3 days to 4 weeks, from 3 days to 3 weeks, from 3 days to 2 weeks, from 3 days to 1 week, from 4 days to 2 months, from 4 days to 1 month, from 4 days to 4 weeks, from 4 days to 3 weeks, from 4 days to 2 weeks, from 4 days to 1 week, from 5 days to 2 months, from 5 days to 1 month, from 5 days to 4 weeks, from 5 days to 3 weeks, from 5 days to 2 weeks, from 5 days to 1 week, from 6 days to 2 months, from 6 days to 1 month, from 6 days to 4 weeks, from 6 days to 3 weeks, from 6 days to 2 weeks, from 1 week to 2 months, from 1 week to 1 month, from 1 to 4 weeks, from 1 to 3 weeks, from 1 to 2 weeks, from 3 weeks to 2 months, from 3 weeks to 1 month, or such as from 3 to 4 weeks prior to administration of the binding agent, prior to administration of a first dosage of the binding agent and/or prior to the first treatment cycle with the binding agent.

The plurality of T-cells may be administered to said subject, or the adoptive T-cell therapy may be provided to said subject, simultaneously with or on the same day as administration of the binding agent, simultaneously with or on the same day as administration of the first dose of the binding agent and/or as part of the first treatment cycle with the binding agent.

The plurality of T-cells may be administered to said subject, or the adoptive T-cell therapy may be provided to said subject, prior to administration of the binding agent, prior to administration of a first dosage of the binding agent and/or prior to the first treatment cycle with the binding agent.

In the method according to the invention
i) administration of the immunogenic composition and administration of the plurality of T-cells, or the adoptive T-cell therapy, and
ii) administration of the binding agent, such as administration of the first dose of the binding agent and/or onset of the first treatment cycle with the binding agent,

may be separated by a time period of 2 days to 2 months, such as 2 days to 1 month, 2 days to 4 weeks, 2 days to 3 weeks, 2 days to 2 weeks, 2 days to 1 week, 3 days to 2 months, 3 days to 1 month, 3 days to 4 weeks, 3 days to 3 weeks, 3 days to 2 weeks, 3 days to 1 week, 4 days to 2 months, 4 days to 1 month, 4 days to 4 weeks, 4 days to 3 weeks, 4 days to 2 weeks, 4 days to 1 week, 5 days to 2 months, 5 days to 1 month, 5 days to 4 weeks, 5 days to 3 weeks, 5 days to 2 weeks, 5 days to 1 week, 6 days to 2 months, such as 6 days to 1 month, 6 days to 4 weeks, 6 days to 3 weeks, 6 days to 2 weeks, 1 week to 2 months, 1 week to 1 month, 1 to 4 weeks, 1 to 3 weeks, 1 to 2 weeks, 3 weeks to 2 months, 3 weeks to 1 month or 3 to 4 weeks.

The immunogenic composition and the plurality of T-cells, or the adoptive T-cell therapy may be administered to the subject from 2 days to 2 months prior to administration of the binding agent, prior to administration of a first dosage of the binding agent and/or prior to the first treatment cycle with the binding agent; such as from 2 days to 1 month, from 2 days to 4 weeks, from 2 days to 3 weeks, from 2 days to 2 weeks, from 2 days to 1 week, from 3 days to 2 months, from 3 days to 1 month, from 3 days to 4 weeks, from 3 days to 3 weeks, from 3 days to 2 weeks, from 3 days to 1 week, from 4 days to 2 months, from 4 days to 1 month, from 4 days to 4 weeks, from 4 days to 3 weeks, from 4 days to 2 weeks, from 4 days to 1 week, from 5 days to 2 months, from 5 days to 1 month, from 5 days to 4 weeks, from 5 days to 3 weeks, from 5 days to 2 weeks, from 5 days to 1 week, from 6 days to 2 months, from 6 days to 1 month, from 6 days to 4 weeks, from 6 days to 3 weeks, from 6 days to 2 weeks, from 1 week to 2 months, from 1 week to 1 month, from 1 to 4 weeks, from 1 to 3 weeks, from 1 to 2 weeks, from 3 weeks to 2 months, from 3 weeks to 1 month, or such as from 3 to 4 weeks prior to administration of the binding agent, prior to administration of a first dosage of the binding agent and/or prior to the first treatment cycle with the binding agent.

It is to be understood that each of the binding agent, the immunogenic composition and, optionally, the adjuvant, the plurality of T-cells and cytokine, such as the interleukin-2 receptor agonist, is provided to the subject in an effective amount. As the skilled person will realize, the efficient dosages and dosage regimens for the binding agent when used in combination with the immunogenic composition according to the invention may depend e.g. on the particular binding agent and the route of administration chosen, as well as the particular tumor or cancer to be treated. It may further depend on several additional factors, including the severity of the subject's symptoms, the subject's size and general health and the response of the subject or the cancer or tumor to the treatment.

The effective amount and dosage regimen may be determined by the persons skilled in the art. For example, an “effective amount” of the binding agent for therapeutic use may be measured by its ability to inhibit or arrest tumor growth, slow the progression of disease or stabilize the disease.

In particular, the effectiveness of treatment according to the invention may be evaluated according to the Response Evaluation Criteria In Solid Tumors; version 1.1 (RECIST Criteria v1.1). The RECIST Criteria are set forth in table 4 below.

TABLE 4
Definition of Response (RECIST Criteria v1.1)

| | Category | Criteria |
|---|---|---|
| Based on | Complete | Disappearance of all target lesions. Any pathological lymph |
| target | Response (CR) | nodes must have reduction in short axis to <10 mm. |
| lesions | Category | Criteria |
| | Partial Response | ≥30% decrease in the sum of the LD of target lesions, taking |
| | (PR) | as reference the baseline sum LD. |
| | Stable Disease | Neither sufficient shrinkage to qualify for PR nor sufficient |
| | (SD) | increase to qualify for PD, taking as reference the smallest |
| | | sum of LDs since the treatment started. |
| | Progressive | ≥20% increase in the sum of the LDs of target lesions, taking |
| | Disease (PD) | as reference the smallest sum of the LDs recorded since the |
| | | treatment started or the appearance of one or more new |
| | | lesions. |
| Based on | CR | Disappearance of all non-target lesions and normalization of |
| non-target | | tumor marker level. All lymph nodes must be non- |
| lesions | | pathological in size (<10 mm short axis). |
| | SD | Persistence of one or more non-target lesion(s) or/and |
| | | maintenance of tumor marker level above the normal limits. |
| | PD | Appearance of one or more new lesions and/or unequivocal |
| | | progression of existing non-target lesions. |

In certain embodiments of the invention, a first dosage of the binding agent is administered to said subject prior to administration of the immunogenic composition and/or the immunogenic composition is administered as part of the second or subsequent treatment schedules with said binding agent.

The method according to the invention may further comprise determining whether there is a T-cell specific response to the immunogenic composition in the subject and/or monitoring any T-cell specific response to the immunogenic composition, optionally including monitoring expansion in said subject of T cells specific to the immunogenic composition.

The target antigen may be an antigen, which is specific for said tumor.

The target antigen may be an antigen which is overexpressed by cells of said tumor or cancer; such as overexpressed when compared with cells of healthy tissue.

In particular, the target antigen may be an antigen, which is expressed exclusively by cells of said tumor or cancer, or may be an antigen overexpressed on immunosuppressive cells within the tumor microenvironment (e.g. TAMs, MDSCs, Treg).

The target antigen may for instance be selected from the group consisting of receptor tyrosine-protein kinase erbB-2 (Her2), such as human Her2 (UniProtKB—P04626); B-lymphocyte antigen CD19, such as human B-lymphocyte antigen CD19 (UniProtKB—P15391); Epithelial cell adhesion molecule (EpCAM), such as human EpCAM (UniProtKB—P16422); Epidermal growth factor receptor (EGFR), such a human EGFR (UniProtKB—P00533); Carcinoembryonic antigen-related cell adhesion molecule 5 (CEA, CEACAM5, CD66e), such as human CEA (UniProtKB—P06731); Myeloid cell surface antigen CD33 (CD33), such as human CD33 (UniProtKB—P20138); Ephrin type-A receptor 2 (EphA2), such as human EphA2 (UniProtKB—P29317); Chondroitin sulfate proteoglycan 4 (MCSP or HMW-MAA), such as human MSCP).

The antigen-binding region that binds to said target antigen may be selected from the group consisting of: an antigen-binding region of Herceptin that binds to Her2, an antigen-binding region of Blinatumomab that binds to CD19, an antigen-binding region of catumaxomab that binds to EpCAM, an antigen-binding region of cetuximab or panitumumab that binds to EGFR, and an antigen-binding region of Lintuzumab that binds to CD33.

The tumor to be treated in the method according to the invention may in particular be a solid tumor.

The tumor or cancer may be selected from the group consisting of breast cancer, prostate cancer, non-small cell lung cancer, bladder cancer, ovarian cancer, gastric cancer, colorectal cancer, esophageal cancer and squamous cell carcinoma of the head & neck, cervical cancer, pancreatic cancer, testis cancer, malignant melanoma, a soft-tissue cancer; e.g. synovial sarcoma.

The tumor to be treated according to the invention may further be selected from the group consisting of melanoma, and adenocarcinoma (e.g. ductal adenocarcinoma).

Alternatively, the tumor to be treated according to the invention may be a hematologic tumor.

The hematologic tumor may be selected from the group consisting of B-cell lymphoma, and chronic lymphatic leukemia or acute lymphatic leukemia.

The tumor to be treated according to the invention may in particular be a so-called ‘cold’ tumor; i.e. a tumor that is are devoid of any (functional) immune infiltrate. A cold tumor may in particular be characterized by lack or paucity of tumor T cell infiltration. The mechanisms responsible for the absence or paucity of T cell infiltration may include lack of tumor antigens, defect in antigen presentation, and absence of T cell activation and deficit of homing into the tumor bed.

Also, the method according to the invention may be particularly effective for treating a tumor characterized by an immunosuppressive tumor microenvironment, which again is characterized by the presence of suppressive immune cells such as regulatory T cells (Tregs), myeloid-derived suppressor cells (MDSCs) and/or M2-type macrophages. The suppressive immune cells may prevent or reduce T-cell infiltration of the tumor and/or may locally suppress T-cell function.

In the binding agent used in the method according to the invention, the antigen-binding region that binds to CD3, may in particular be one that binds to human CD3ε (epsilon), such as human CD3ε (epsilon) as specified in SEQ ID NO: 1.

In particular embodiments of the invention, the antigen-binding region that binds to CD3 comprises
a heavy chain variable region (VH) comprising the CDR1, CDR2, and CDR3 sequences of SEQ ID NOs.: 2, 3 and 4, respectively; [wildtype anti-CD3 (SP34/humanized SP34, WO2015001085 (Genmab))—VH CDR sequences];

and, optionally
a light chain variable region (VL) comprising the CDR1, CDR2, and CDR3 sequences of SEQ ID NO: 6, GTN and 7, respectively [wildtype anti-CD3, VL CDR sequences].

The antigen-binding region that binds to CD3 may comprises
a heavy chain variable region (VH) comprising the sequence of SEQ ID NO: 57, or a sequence having at least 90%, at least 95%, at least 97%, or at least 99% amino acid sequence identity to the sequence of SEQ ID NO: 5 [wildtype anti-CD3—VH full length sequence];

and, optionally
a light chain variable region (VL) comprising the sequence of SEQ ID NO: 60 or a sequence having at least 90%, at least 95%, at least 97%, or at least 99% amino acid sequence identity to the sequence of SEQ ID NO: 8, [wild type anti-CD3—VL full length sequence].

The binding agent used in the method according to the invention may be one that has a lower human CD3ε binding affinity than a binding agent having an antigen-binding region comprising a VH sequence as set forth in SEQ ID NO: 5, and a VL sequence as set forth in SEQ ID NO: 8 [wild type anti-CD3 (humanized SP34, WO2015001085 (Genmab)) VH and VL sequences], preferably wherein said affinity is at least 5-fold lower, such as at least 10-fold lower, e.g. at least 20-fold lower, at least 30 fold lower, at least 40 fold lower, at least 45 fold lower or such as at least 50-fold lower.

The said antigen-binding region that binds to CD3 may be one that binds with an equilibrium dissociation constant K_{D} within the range of 200-1000 nM, such as within the range of 300-1000 nM, within the range of 400-1000 nM, within the range of 500-1000 nM, within the range of 300-900 nM within the range of 400-900 nM, within the range of 400-700 nM, within the range of 500-900 nM, within the range of 500-800 nM, within the range of 500-700 nM, within the range of 600-1000 nM, within the range of 600-900 nM, within the range of 600-800 nM, or such as within the range of 600-700 nM.

The antigen binding-region that binds to CD3, may be one that binds with an equilibrium dissociation constant K_{D} within the range of 1-100 nM, such as within the range of 5-100 nM, within the range of 10-100 nM, within the range of 1-80 nM, within the range of 1-60 nM within the range of 1-40 nM, within the range of 1-20 nM, within the range of 5-80 nM, within the range of 5-60 nM, within the range of 5-40 nM, within the range of 5-20 nM, within the range of 10-80 nM, within the range of 10-60 nM, within the range of 10-40 nM, or such as within the range of 10-20 nM.

In some embodiments of the invention, the antigen binding region that binds to CD3 comprises a heavy chain variable (VH) region comprising a CDR1 sequence, a CDR2 sequence and a CDR3 sequence, wherein
the heavy chain variable (VH) region, when compared to a heavy chain variable (VH) region comprising the sequence set forth in SEQ ID NO: 5, has an amino acid substitution in one of the CDR sequences, the substitution being at a position selected from the group consisting of: T31, N57, H101, G105, S110 and Y114, the positions being numbered according to the sequence of SEQ ID NO: 5 [VH_huCD3-H1L1]; and
the wild type light chain variable (VL) region comprises the CDR1, CDR2 and CDR3 sequences set forth in SEQ ID NO: 6, GTN and SEQ ID NO: 7, respectively.

The CDR1, CDR2 and CDR3 of the heavy chain variable (VH) region of the antigen binding region that binds to CD3 may comprise, in total, at the most 1, 2, 3, 4 or 5 amino acid substitutions, when compared to the CDR1, CDR2 and CDR3 of the sequence set forth in SEQ ID NO: 5.

The amino acid sequences of the CDR1, CDR2 and CDR3 of the heavy chain variable (VH) region of the antigen-binding region that binds to CD3 may have at least 95% sequence identity, such as at least 96% sequence identity, at least 97% sequence identity, at least 98% sequence identity or at least 99% sequence identity to the amino acid sequences of the CDR1, CDR2 and CDR3 of the wild type heavy chain variable (VH) region, sequence identity being calculated based on an aligning an amino acid sequence consisting of the sequences of the CDR1, CDR2 and CDR3 of the heavy chain variable (VH) region of the antigen binding region that binds to CD with an amino acid sequence comprising the sequences of the CDR1, CDR2 and CDR3 of the wild type heavy chain variable (VH) region.

The antigen-binding region that binds to CD3 may in particular comprise a mutation selected from the group consisting of: T31M, T31P, N57E, H101G, H101N, G105P, S110A, S110G, Y114M, Y114R, Y114V.

The antigen-binding region capable of binding to CD3 may comprise:
a) a heavy chain variable region (VH) comprising CDR1, CDR2, and CDR3 having the sequences as set forth in SEQ ID NOs: 9, 3, and 4 [VH CDR1-T31P+Wild type VH CDRs 2,3], respectively, and a light chain variable region (VL) comprising CDR1, CDR2, and CDR3 having the sequences as set forth in SEQ ID NO: 6, the sequence GTN, and the sequence as set forth in SEQ ID NO: 7, respectively [Wild type VL CDRs 1,2,3], or
b) a heavy chain variable region (VH) comprising CDR1, CDR2, and CDR3 having the sequences as set forth in SEQ ID NOs: 11, 3, and 4 [VH CDR1-T31M+Wild type VH CDRs 2,3], respectively, and a light chain variable region (VL) comprising CDR1, CDR2, and CDR3 having the sequences as set forth in SEQ ID NO: 6, the sequence GTN, and the sequence as set forth in SEQ ID NO: 7, respectively [Wild type VL CDRs 1,2,3], respectively, or
c) a heavy chain variable region (VH) comprising CDR1, CDR2, and CDR3 having the sequences as set forth in SEQ ID NOs: 2, 13, and 4 [VH CDR-N57E+Wild type VH CDRs 1,3], respectively, and a light chain variable region (VL) comprising CDR1, CDR2, and CDR3 having the sequences as set forth in SEQ ID NO: 6, the sequence GTN, and the sequence as set forth in SEQ ID NO: 7, respectively [Wild type VL CDRs 1,2,3], respectively, or
d) a heavy chain variable region (VH) comprising CDR1, CDR2, and CDR3 having the sequences as set forth in SEQ ID NOs: 2, 3, and 15 [Wild type VH CDRs 1,2+VH CDR3-H101G], respectively, and a light chain variable region (VL) comprising CDR1, CDR2, and CDR3 having the sequences as set forth in SEQ ID NO: 6, the sequence GTN, and the sequence as set forth in SEQ ID NO: 7, respectively [Wild type VL CDRs 1,2,3], respectively.
e) a heavy chain variable region (VH) comprising CDR1, CDR2, and CDR3 having the sequences as set forth in SEQ ID NOs: 2, 3, and 17 [Wild type VH CDRs 1,2+VH CDR3-H101N], respectively, and a light chain variable region (VL) comprising CDR1, CDR2, and CDR3 having the sequences as set forth in SEQ ID NO: 6, the sequence GTN, and the sequence as set forth in SEQ ID NO: 7, respectively [Wild type VL CDRs 1,2,3], respectively;
f) a heavy chain variable region (VH) comprising CDR1, CDR2, and CDR3 having the sequences as set forth in SEQ ID NOs: 2, 3, and 19 [Wild type VH CDRs 1,2+VH CDR3-G105P], respectively, and a light chain variable region (VL) comprising CDR1, CDR2, and CDR3 having the sequences as set forth in SEQ ID NO: 6, the sequence GTN, and the sequence as set forth in SEQ ID NO: 7, respectively [Wild type VL CDRs 1,2,3], respectively;
g) a heavy chain variable region (VH) comprising CDR1, CDR2, and CDR3 having the sequences as set forth in SEQ ID NOs: 2, 3, and 21 [Wild type VH CDRs 1,2+VH CDR3-S110A], respectively, and a light chain variable region (VL) comprising CDR1, CDR2, and CDR3 having the sequences as set forth in SEQ ID NO: 6, the sequence GTN, and the sequence as set forth in SEQ ID NO: 7, respectively [Wild type VL CDRs 1,2,3], respectively, or
h) a heavy chain variable region (VH) comprising CDR1, CDR2, and CDR3 having the sequences as set forth in SEQ ID NOs: 2, 3, and 23 [Wild type VH CDRs 1,2+VH CDR3-S110G], respectively, and a light chain variable region (VL) comprising CDR1, CDR2, and CDR3 having the sequences as set forth in SEQ ID NO: 658, the sequence GTN, and the sequence as set forth in SEQ ID NO: 7, respectively [Wild type VL CDRs 1,2,3], respectively,
i) a heavy chain variable region (VH) comprising CDR1, CDR2, and CDR3 having the sequences as set forth in SEQ ID NOs: 2, 3, and 25 [Wild type VH CDRs 1,2+VH CDR3-Y114V], respectively, and a light chain variable region (VL) comprising CDR1, CDR2, and CDR3 having the sequences as set forth in SEQ ID NO: 6, the sequence GTN, and the sequence as set forth in SEQ ID NO: 7, respectively [Wild type VL CDRs 1,2,3], respectively, or
j) a heavy chain variable region (VH) comprising CDR1, CDR2, and CDR3 having the sequences as set forth in SEQ ID NOs: 2, 3, and 27 [Wild type VH CDRs 1,2+VH CDR3-Y114M], respectively, and a light chain variable region (VL) comprising CDR1, CDR2, and CDR3 having the sequences as set forth in SEQ ID NO: 6, the sequence GTN, and the sequence as set forth in SEQ ID NO: 7, respectively [Wild type VL CDRs 1,2,3], respectively, or
k) a heavy chain variable region (VH) comprising CDR1, CDR2, and CDR3 having the sequences as set forth in SEQ ID NOs: 2, 3, and 29 [Wild type VH CDRs 1,2+VH CDR3-Y114R], respectively, and a light chain variable region (VL) comprising CDR1, CDR2, and CDR3 having the sequences as set forth in SEQ ID NO: 6, the sequence GTN, and the sequence as set forth in SEQ ID NO: 7, respectively [Wild type VL CDRs 1,2,3], respectively.

The antigen-binding region capable of binding to CD3 may comprise a heavy chain variable region (VH) comprising CDR1, CDR2, and CDR3 having the sequences as set forth in SEQ ID NOs: 2, 3, and 15 [Wild type VH CDRs 1,2+VH CDR3-H101G], respectively, and a light chain variable region (VL) comprising CDR1, CDR2, and CDR3 having the sequences as set forth in SEQ ID NO: 6, the sequence GTN, and the sequence as set forth in SEQ ID NO: 7, respectively [Wild type VL CDRs 1,2,3], respectively.

The antigen-binding region capable of binding to human CD3 may comprise a VH sequence and a VL sequence selected from the group consisting of:
a) a VH sequence as set forth in SEQ ID NO: 10 [VH T31P full length sequence] and a VL sequence as set forth in SEQ ID NO: 8 [Wild type full length sequence],
b) a VH sequence as set forth in SEQ ID NO: 12 [VH T31M full length sequence] and a VL sequence as set forth in SEQ ID NO: 8,
c) a VH sequence as set forth in SEQ ID NO: 14 [VH N57E full length sequence] and a VL sequence as set forth in SEQ ID NO: 8,
d) a VH sequence as set forth in SEQ ID NO: 16 [VH H101G full length sequence] and a VL sequence as set forth in SEQ ID NO: 8,
e) a VH sequence as set forth in SEQ ID NO: 18 [VH H101N full length sequence] and a VL sequence as set forth in SEQ ID NO: 8,
f) a VH sequence as set forth in SEQ ID NO: 20 [VH G105P full length sequence] and a VL sequence as set forth in SEQ ID NO: 8,
g) a VH sequence as set forth in SEQ ID NO: 22 [VH S110A full length sequence] and a VL sequence as set forth in SEQ ID NO: 8,
h) a VH sequence as set forth in SEQ ID NO: 24 [VH S110G full length sequence] and a VL sequence as set forth in SEQ ID NO: 8,
i) a VH sequence as set forth in SEQ ID NO: 26 [VH Y114V full length sequence] and a VL sequence as set forth in SEQ ID NO: 8,
j) a VH sequence as set forth in SEQ ID NO: 28 [VH Y114M full length sequence] and a VL sequence as set forth in SEQ ID NO: 8; and
k) a VH sequence as set forth in SEQ ID NO: 30 [VH Y114R full length sequence] and a VL sequence as set forth in SEQ ID NO: 8.

It is currently preferred that the antigen-binding region capable of binding to human CD3 comprises a VH sequence as set forth in SEQ ID NO: 10 [VH H101G full length sequence] and a VL sequence as set forth in SEQ ID NO: 8.

It is further preferred that the binding agent is an antibody, such as an antibody of an isotype subclass selected from the group consisting of IgG1, IgG2, IgG3 and IgG4.

The binding agent may be a full-length antibody, such as a full length IgG1 antibody.

In one embodiment, the antibody is a human antibody or is an antibody in which all variable and constant regions are those of a human antibody. Alternatively, the antibody may be a humanized antibody or an antibody in which all variable regions are from a humanized antibody.

The antibody may also have a 1^{st} arm comprising the binding region that binds to CD3, and a 2^{nd} arm comprising the binding region that binds to the target antigen, wherein the 1^{st} binding arm is that of a human antibody and the 2^{nd} binding arm is that of a humanized antibody or vice versa.

The binding agent may be an antibody of the IgG1m(f) allotype.

The binding agent may in particular be a multi-specific antibody, such as a bispecific antibody.

Examples of bispecific antibody molecules which may be used in the present invention comprise (i) a single antibody that has two arms comprising different antigen-binding regions, (ii) a single chain antibody that has specificity to two different epitopes, e.g., via two scFvs linked in tandem by an extra peptide linker; (iii) a dual-variable-domain antibody (DVD-Ig™), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (iv) a chemically-linked bispecific (Fab′)₂ fragment; (v) a Tandab®, which is a fusion of two single chain diabodies resulting in a tetravalent bispecific antibody that has two binding sites for each of the target antigens; (vi) a flexibody, which is a combination of scFvs with a diabody resulting in a multivalent molecule; (vii) a so called “dock and lock” molecule (Dock-and-Lock®), based on the “dimerization and docking domain” in Protein Kinase A, which, when applied to Fabs, can yield a trivalent bispecific binding protein consisting of two identical Fab fragments linked to a different Fab fragment; (viii) a so-called Scorpion molecule, comprising, e.g., two scFvs fused to both termini of a human Fab-arm; and (ix) a diabody.

In one embodiment, a bispecific antibody used according to the present invention is a diabody, a cross-body, such as CrossMabs, or a bispecific antibody obtained via a controlled Fab arm exchange such as described in (WO 2011/131746; Genmab A/S).

Examples of different classes of bispecific antibodies include but are not limited to (i) IgG-like molecules with complementary CH3 domains to force heterodimerization; (ii) recombinant IgG-like dual targeting molecules, wherein the two sides of the molecule each contain the Fab fragment or part of the Fab fragment of at least two different antibodies; (iii) IgG fusion molecules, wherein full length IgG antibodies are fused to extra Fab fragment or parts of Fab fragment; (iv) Fc fusion molecules, wherein single chain Fv molecules or stabilized diabodies are fused to heavy-chain constant-domains, Fc-regions or parts thereof; (v) Fab fusion molecules, wherein different Fab-fragments are fused together, fused to heavy-chain constant-domains, Fc-regions or parts thereof; and (vi) ScFv- and diabody-based and heavy chain antibodies (e.g., domain antibodies, Nanobodies®) wherein different single chain Fv molecules or different diabodies or different heavy-chain antibodies (e.g. domain antibodies, Nanobodies®) are fused to each other or to another protein or carrier molecule fused to heavy-chain constant-domains, Fc-regions or parts thereof.

Examples of IgG-like molecules with complementary CH3 domains molecules include but are not limited to the Triomab® (Trion Pharma/Fresenius Biotech, WO/2002/020039; Trion Pharma/Fresenius Biotech), the Knobs-into-Holes (Genentech, WO2011117329; Roche), CrossMAbs (Roche, [33]) and the electrostatically-matched (Amgen, EP1870459; Amgen and WO2009089004; Amgen; Chugai, [US201000155133; Chugai; Oncomed, WO2010129304; Oncomed), the LUZ-Y (Genentech), DIG-body and PIG-body (Pharmabcine), the Strand Exchange Engineered Domain body (SEEDbody)(EMD Serono, WO2007110205; EMD Serono), the Biclonics (Merus), FcAAdp (Regeneron, WO 2010/015792; Regeneron), bispecific IgG1 and IgG2 (Pfizer/Rinat, WO11143545; Pfizer/Rinat), Azymetric scaffold (Zymeworks/Merck, WO2012058768: Zymeworks/Merck), mAb-Fv (Xencor, WO2011028952; Xencor), bivalent bispecific antibodies (Roche WO 2009/080254; Roche) and DuoBody® molecules (Genmab A/S, WO 2011/131746; Genmab A/S).

Examples of recombinant IgG-like dual targeting molecules include but are not limited to Dual Targeting (DT)-Ig (GSK/Domantis), Two-in-one Antibody (Genentech), Cross-linked Mabs (Karmanos Cancer Center), mAb2 (F-Star, [44]), Zybodies™ (Zyngenia), approaches with common light chain (Crucell/Merus, [45]), KXBodies (Novlmmune) and CovX-body (CovX/Pfizer).

Examples of IgG fusion molecules include but are not limited to Dual Variable Domain (DVD)-Ig™ (Abbott, U.S. Pat. No. 7,612,181; Abbott, Dual domain double head antibodies (Unilever; Sanofi Aventis, WO20100226923; Unilever, Sanofi Aventis), IgG-like Bispecific (ImClone/Eli Lilly), Ts2Ab (MedImmune/AZ) and BsAb (Zymogenetics), HERCULES (Biogen Idec, US007951918; Biogen Idec), scFv fusion (Novartis), scFv fusion (Changzhou Adam Biotech Inc, CN 102250246; Changzhou Adam Biotech Inc) and TvAb (Roche, WO2012025525; Roche, WO2012025530; Roche).

Examples of Fc fusion molecules include but are not limited to ScFv/Fc Fusions (Academic Institution), SCORPION (Emergent BioSolutions/Trubion, Zymogenetics/BMS), Dual Affinity Retargeting Technology (Fc-DART™) (MacroGenics, WO2008157379; Macrogenics, WO2010/080538; Macrogenics and Dual(ScFv)2-Fab (National Research Center for Antibody Medicine—China).

Examples of Fab fusion bispecific antibodies include but are not limited to F(ab)₂ (Medarex/AMGEN), Dual-Action or Bis-Fab (Genentech), Dock-and-Lock® (DNL) (ImmunoMedics), Bivalent Bispecific (Biotecnol) and Fab-Fv (UCB-Celltech).

Examples of ScFv-, diabody-based and domain antibodies include but are not limited to BispecificT Cell Engager (BiTE®) (Micromet, Tandem Diabody (Tandab™) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting Nanobodies® (Ablynx), dual targeting heavy chain only domain antibodies.

In the binding agent used according to the invention each antigen-binding region may comprise a heavy chain variable region (VH) and a light chain variable region (VL), and wherein said variable regions each comprise three CDR sequences, CDR1, CDR2 and CDR3, respectively, and four framework sequences, FR1, FR2, FR3 and FR4, respectively.

The binding agent may comprise two heavy chain constant regions (CH), and two light chain constant regions (CL).

The binding agent used in the method according to the invention may comprise a first and a second heavy chain, each of said first and second heavy chain comprises at least a hinge region, a CH2 and CH3 region, wherein in said first heavy chain at least one of the amino acids in the positions corresponding to positions selected from the group consisting of T366, L368, K370, D399, F405, Y407 and K409 in a human IgG1 heavy chain has been substituted, and in said second heavy chain at least one of the amino acids in the positions corresponding to a position selected from the group consisting of T366, L368, K370, D399, F405, Y407, and K409 in a human IgG1 heavy chain has been substituted, wherein said substitutions of said first and said second heavy chains are not in the same positions, and wherein the amino acid positions are numbered according to EU numbering.

The amino acid in the position corresponding to K409 in a human IgG1 heavy chain may in particular be R in said first heavy chain, and the amino acid in the position corresponding to F405 in a human IgG1 heavy chain may in particular be L in said second heavy chain, or vice versa.

The binding agent used according to the invention may be a binding agent that comprises a first and a second heavy chain, and wherein in both the first and the second heavy chain, the amino acid residues at the positions corresponding to positions L234 and L235 in a human IgG1 heavy chain according to EU numbering are F and E, respectively.

The binding agent used according to the invention may be a binding agent that comprises a first and a second heavy chain, and wherein in both the first and the second heavy chain, the amino acid residue at the position corresponding to position D265 in a human IgG1 heavy chain according to Eu numbering is A.

The binding agent used according to the invention may also be a binding agent that comprises a first and a second heavy chain, and wherein in both the first and the second heavy chain, the amino acid residue at the position corresponding to position G236 in a human IgG1 heavy chain according to Eu numbering is R.

The binding agent used according to the invention may be a binding agent that comprises a first and, optionally, a second heavy chain and wherein the first heavy chain, and the second heavy chain if present, is/are modified so that the antibody induces Fc-mediated effector function to a lesser extent relative to an identical non-modified antibody.

The binding agent may comprise a kappa (κ) light chain.

Alternatively, the binding agent may comprise a lambda (λ) light chain.

In particular, the binding agent may comprise a heavy chain and a lambda (λ) light chain, which comprise the binding region that binds to CD3.

Alternatively, the binding agent may comprise a heavy chain and a kappa (κ) light chain, which comprise the binding region that binds to CD3.

When included in the binding agent used according to the invention, the kappa (κ) light chain comprise an amino acid sequence selected from the group consisting of
a) the sequence set forth in SEQ ID NO: 31,
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 5 substitutions, such as at the most 4 substitutions, at the most 3, at the most 2 or at the most 1 substitution compared to the amino acid sequence defined in a) or b).

When included in the binding agent used according to the invention, the lambda (λ) light chain may comprise an amino acid sequence selected from the group consisting of
a) the sequence set forth in SEQ ID NO: 32,
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 5 substitutions, such as at the most 4 substitutions, at the most 3, at the most 2 or at the most 1 substitution compared to the amino acid sequence defined in a) or b).

The constant region of said first and/or second heavy chain may comprise or consists essentially of or consists of an amino acid sequence selected from the group consisting of
a) the sequence set forth in SEQ ID NO: 33,
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 5 substitutions, such as at the most 4 substitutions, at the most 3, at the most 2 or at the most 1 substitution compared to the amino acid sequence defined in a) or b).

The at the most 5 substitutions may comprise one or more substitutions, such as 1, 2, 3 or 4 substitutions, selected from the group consisting of L234F, L235E, D265A, F405L and K409R.

Alternatively, the at the most 5 substitutions may comprise one or more substitutions, such as 1, 2, 3 or 4 substitutions, selected from the group consisting of L234F, L235E, G236R, F405L and K409R.

The invention further provides a method for preventing or reducing growth of a tumor in a subject in need thereof, comprising
providing to the subject an immunogenic composition comprising at least one vaccine antigen, thereby increasing the relative amount of immune cells within the live cell population in the tumor, and optionally increasing the activation of said immune cells;
and
providing to the subject a binding agent comprising an antigen-binding region that binds to CD3, such as human CD3, and an antigen-binding region that binds to a target antigen on cells of said tumor or cancer.

The immune cells are selected from the group consisting of T cells, such as CD8⁺ T cells, natural killer (NK) cells and natural killer T (NKT) cells.

In an alternative approach, the binding agent used in the method according to the invention may be administered to the subject by gene delivery. Hence, a 2^{nd} aspect the present invention provides a method for preventing or reducing growth of a tumor, or for treatment of cancer in a subject in need thereof, comprising providing to the subject
i) a nucleic acid construct encoding a binding agent comprising an antigen-binding region that binds to CD3, such as human CD3, and an antigen-binding region that binds to a target antigen on cells of said tumor or cancer; and
ii) an immunogenic composition comprising at least one vaccine antigen.

Different strategies may be utilized for efficient gene delivery of the binding agent, including the use of a vector such as an adeno-associated virus (AAV) vector to deliver the polynucleotide(s) encoding the binding agent. In an alternative approach, one may deliver mRNA encoding the binding agent via a nanoparticle formulation e.g. lipid-based, peptide-based, polysaccharide-based, or inorganic nanoparticles. The mRNA may be delivered directly into the organ(s) where expression of the binding agent is desired, or delivered systemically via injection or infusion, which would generate expression over time (starting within hours and lasting up until several days). In the latter approach, the mRNA may be optimized by one or more means to prevent immune activation, increase stability, reduce any tendency to aggregate over time, and/or to avoid impurities Such optimization may include the use of modified nucleosides (for example, with I-methylpseudouridine) in the mRNA and/or may include particular 5′TRs, 3′UTRs, and/or poly(A) tail for improved intracellular stability and translational efficiency (see, e.g., Stadler Nat Med. 2017; 23(7):815-817).

It is to be understood that the binding agent delivered by gene therapy may have any of the characteristics set forth above in the context a protein-based delivery approach. Also, the immunogenic composition may be a composition as disclosed above.

Further, it will be understood that the features disclosed in relation to the method according to the first aspect of the invention apply equally well to the method of the 2^{nd} aspect of the invention. In particular, the nucleic acid construct encoding the binding agent may be administered in combination with an adjuvant, such as a TLR-agonist, as disclosed above. It may also be administered in further combination with a cytokine such as an interleukin-2 receptor agonist, e.g. human interleukin-2 or human interleukin-15 or an analog thereof, all as provided above.

In a 3^{rd} aspect, the present invention provides a binding agent for use in preventing or reducing tumor outgrowth or treatment of cancer in a subject, the binding agent comprising a binding region that binds to CD3 and a binding region that binds to a target on cells of said tumor or cancer, wherein the use comprises providing the antibody to the subject in combination with an immunogenic composition comprising at least one vaccine antigen.

The binding agent for use according to the invention may be as defined above in relation to the method according to the invention.

Also, in relation to this aspect of the invention, the immunogenic composition may be as defined above in relation to the method according to the invention.

Again, it will be understood that the features disclosed in relation to the method according to the first aspect of the invention apply equally well to the use according to 3^{rd} aspect of the invention. In particular, the binding agent may be administered in combination with an adjuvant as disclosed above. It may also be administered in further combination with a cytokine such as an interleukin-2 receptor agonist, e.g. human interleukin-2, human interleukin-15 or an analog thereof, all as provided above.

In particular, the binding agent is administered in further combination with a cytokine such as an interleukin-2 receptor agonist, e.g. human interleukin 2, human interleukin-15 or an analog thereof.

Also, as disclosed hereinbefore, the binding agent may be administered in further combination with an adjuvant, such as an adjuvant as defined above.

Alternatively, the binding agent may be administered in combinaton with an immunogeneic composition for gene based therapy; e.g. comprisng an immunogenic nucleic acid sequence or a lipid nanoparticle (LNP).

A 4^{th} aspect of the invention relates to an immunogenic composition comprising at least one vaccine antigen for use in preventing or reducing tumor outgrowth or treatment of cancer in a subject, wherein the immunogenic composition is provided to the subject in combination with a binding agent comprising a binding region that binds to CD3 and a binding region that binds to a target on cells of said tumor or cancer or to a target expressed on immunosuppressive cells within the tumor microenvironment.

The immunogenic composition may have any one or more of the features and characteristics defined above.

In relation to the immunogenic composition for use according to the invention, the binding agent may be a binding agent as defined in relation to the 1^{st} aspect of the invention.

In addition, any one or more of the features disclosed in relation to the method according to the first aspect of the invention apply equally well to the use according to 4^{th} aspect of the invention. In particular, the immunogenic composition and the binding agent may be administered in further combination with an adjuvant as disclosed above. It may also be administered in further combination with a cytokine such as an interleukin-2 receptor agonist, e.g. human interleukin-2, human interleukin-15 or an analog thereof, all as provided above.

A 5^{th} aspect of the invention relates to the use of a binding agent in the manufacture of a medicament for treatment of cancer in a subject, the binding agent comprising a binding region that binds to CD3 and a binding region that binds to a target on cells of said tumor or cancer, wherein the treatment comprises providing said binding agent to the subject in combination with an immunogenic composition comprising at least one vaccine antigen.

The binding agent and the immunogenic composition may be is as defined in relation to the 1^{st} aspect of the invention.

Again, any one or more of the features disclosed in relation to the method according to the first aspect of the invention apply equally well to the use according to 5^{th} aspect of the invention. In particular the immunogenic composition and the binding agent may be administered in further combination with an adjuvant as disclosed above. It may also be administered in further combination with a cytokine such as an interleukin 2 receptor agonist, e.g. human interleukin 2 or an analog thereof, all as provided above.

The present invention further provides, in a 6^{th} aspect, the use of an immunogenic composition comprising at least one vaccine antigen in the manufacture of a medicament for treatment of cancer in a subject, wherein the immunogenic composition is provided to the subject in combination with a binding agent comprising a binding region that binds to CD3 and a binding region that binds to a target on cells of said tumor or cancer.

The immunogenic composition and the binding agent may have any of the features and characteristics defined in relation to the 1^{st} aspect of the invention.

In addition, any one or more of the features disclosed in relation to the method according to the 1^{st} aspect of the invention apply equally well to the use according to 4^{th} aspect of the invention. In particular the immunogenic composition and the binding agent may be administered in further combination with an adjuvant as disclosed above. It may also be administered in further combination with a cytokine such as an interleukin-2 receptor agonist, e.g. human interleukin-2, human interleukin-15 or an analog thereof, all as provided above.

In a final aspect of the invention, a kit of parts is provided, comprising a binding agent comprising a binding region that binds to CD3 and a binding region that binds to a target on cells of a tumor or cancer, and an immunogenic composition comprising at least one vaccine antigen.

The kit of parts may further comprise an adjuvant, such as an adjuvant as defined above.

The kit of parts may further comprise an amount of a cytokine as disclosed in relation to the 1^{st} aspect of the invention, such interleukin-2 or an interleukin-2 receptor agonist.

In addition, any one or more of the features disclosed in relation to the method according to the 1^{st} aspect of the invention apply equally well to the constituents of the kit of parts provided according to the invention and the way they are used for therapeutic purposes. In particular, the immunogenic composition and the binding agent may be administered in further combination with an adjuvant as disclosed above. It may also be administered in further combination with a cytokine such as an interleukin-2 receptor agonist, e.g. human interleukin-2 or an analog thereof, all as provided above.

The kit of parts may further comprise instructions for use, such as for administration of the binding agent in combination with the immunogenic composition. The instructions may also provide directions to further combine the binding agent and the immunogenic composition with, or administer the binding agent and the immunogenic composition in combination with an adjuvant as defined above and/or with a cytokine as defined above.

TABLE 5
sequences

| SEQ ID | | | |
|---|---|---|---|
| No. | Name | Sequence | Species |
| 1 | Mature Human | QDGNEEMGGITQTPYKVSISGTTVILTCPQYPGSEILWQHN | Human |
| | CD3ε (epsilon) | DKNIGGDEDDKNIGSDEDHLSLKEFSELEQSGYYVCYPRGSK | |
| | protein | PEDANFYLYLRARVCENCMEMDVMSVATIVIVDICITGGLLL | |
| | | LVYYWSKNRKAKAKPVTRGAGAGGRQRGQNKERPPPVPN | |
| | | PDYEPIRKGQRDLYSGLNQRRI | |
| 2 | VH_huCD3- | GFTFNTYA | Humanized |
| | H1L1_CDR1 | | |
| 3 | VH_huCD3- | IRSKYNNYAT | Humanized |
| | H1L1_CDR2 | | |
| 4 | VH_huCD3- | VRHGNFGNSYVSWFAY | Humanized |
| | H1L1_CDR3 | | |
| 5 | VH_huCD3- | EVKLVESGGGLVQPGGSLRLSCAASGFTFNTYAMNWVRQ | Humanized |
| | H1L1 | APGKGLEWVARIRSKYNNYATYYADSVKDRFTISRDDSKSSL | |
| | | YLQMNNLKTEDTAMYYCVRHGNFGNSYVSWFAYWGQGT | |
| | | LVTVSS | |
| 6 | VL_huCD3- | TGAVTTSNY | Humanized |
| | H1L1_CDR1 | | |
| | VL_huCD3- | GTN | Humanized |
| | H1L1_CDR2 | | |
| 7 | VL_huCD3- | ALWYSNLWV | Humanized |
| | H1L1_CDR3 | | |
| 8 | VL_huCD3-H1L1 | QAVVTQEPSFSVSPGGTVTLTCRSSTGAVTTSNYANWVQQ | Humanized |
| | | TPGQAFRGLIGGTNKRAPGVPARFSGSLIGDKAALTITGAQ | |
| | | ADDESIYFCALWYSNLWVFGGGTKLTVL | |
| 9 | VH CDR1-T31P | GFTFNPYA | Humanized |
| | HC_T31P CDR1 | | |
| 10 | VH T31P full | EVKLVESGGGLVQPGGSLRLSCAASGFTFNPYAMNWVRQ | Humanized |
| | length sequence | APGKGLEWVARIRSKYNNYATYYADSVKDRFTISRDDSKSSL | |
| | HC_T31P | YLQMNNLKTEDTAMYYCVRHGNFGNSYVSWFAYWGQGT | |
| | | LVTVSS | |
| 11 | VH CDR1-T31M | GFTFNMYA | Humanized |
| | HC_T31M CDR1 | | |
| 12 | VH T31M full | EVKLVESGGGLVQPGGSLRLSCAASGFTFNMYAMNWVRQ | Humanized |
| | length sequence | APGKGLEWVARIRSKYNNYATYYADSVKDRFTISRDDSKSSL | |
| | HC_T31M | YLQMNNLKTEDTAMYYCVRHGNFGNSYVSWFAYWGQGT | |
| | | LVTVSS | |
| 13 | VH CDR2-N57E | IRSKYNEYAT | Humanized |
| | HC_N57E_CDR2 | | |
| 14 | VH N57E full | EVKLVESGGGLVQPGGSLRLSCAASGFTFNTYAMNWVRQ | Humanized |
| | length sequence | APGKGLEWVARIRSKYNEYATYYADSVKDRFTISRDDSKSSL | |
| | HC_N57E | YLQMNNLKTEDTAMYYCVRHGNFGNSYVSWFAYWGQGT | |
| | | LVTVSS | |
| 15 | VH_huCD3- | VRGGNFGNSYVSWFAY | Humanized |
| | H1L1- | | |
| | H101G_CDR3 | | |
| | HC_H101G | | |
| | CDR3 | | |
| 16 | VH_huCD3- | EVKLVESGGGLVQPGGSLRLSCAASGFTFNTYAMNWVRQ | Humanized |
| | H1L1-H101G | APGKGLEWVARIRSKYNNYATYYADSVKDRFTISRDDSKSSL | |
| | HC_H101G | YLQMNNLKTEDTAMYYCVRGGNFGNSYVSWFAYWGQGT | |
| | | LVTVSS | |
| 17 | VH CDR3-H101N | VRNGNFGNSYVSWFAY | Humanized |
| 18 | VH H101N full | EVKLVESGGGLVQPGGSLRLSCAASGFTFNTYAMNWVRQ | Humanized |
| | length sequence | APGKGLEWVARIRSKYNNYATYYADSVKDRFTISRDDSKSSL | |
| | | YLQMNNLKTEDTAMYYCVRNGNFGNSYVSWFAYWGQGT | |
| | | LVTVSS | |
| 19 | VH CDR3-G105P | VRHGNFPNSYVSWFAY | Humanized |
| 20 | VH G105P full | EVKLVESGGGLVQPGGSLRLSCAASGFTFNTYAMNWVRQ | Humanized |
| | length sequence | APGKGLEWVARIRSKYNNYATYYADSVKDRFTISRDDSKSSL | |
| | | YLQMNNLKTEDTAMYYCVRHGNFPNSYVSWFAYWGQGT | |
| | | LVTVSS | |
| 21 | VH CDR3-S110A | VRHGNFGNSYVAWFAY | Humanized |
| 22 | VH S110A full | EVKLVESGGGLVQPGGSLRLSCAASGFTFNTYAMNWVRQ | Humanized |
| | length sequence | APGKGLEWVARIRSKYNNYATYYADSVKDRFTISRDDSKSSL | |
| | | YLQMNNLKTEDTAMYYCVRHGNFGNSYVAWFAYWGQG | |
| | | TLVTVSS | |
| 23 | VH CDR3-S110G | VRHGNFGNSYVGWFAY | Humanized |
| 24 | VH S110G full | EVKLVESGGGLVQPGGSLRLSCAASGFTFNTYAMNWVRQ | Humanized |
| | length sequence | APGKGLEWVARIRSKYNNYATYYADSVKDRFTISRDDSKSSL | |
| | | YLQMNNLKTEDTAMYYCVRHGNFGNSYVGWFAYWGQG | |
| | | TLVTVSS | |
| 25 | VH CDR3-Y114V | VRHGNFGNSYVSWFAV | Humanized |
| 26 | VH Y114V full | EVKLVESGGGLVQPGGSLRLSCAASGFTFNTYAMNWVRQ | Humanized |
| | length sequence | APGKGLEWVARIRSKYNNYATYYADSVKDRFTISRDDSKSSL | |
| | | YLQMNNLKTEDTAMYYCVRHGNFGNSYVSWFAVWGQGT | |
| | | LVTVSS | |
| 27 | VH CDR3-Y114M | VRHGNFGNSYVSWFAM | Humanized |
| 28 | VH Y114M full | EVKLVESGGGLVQPGGSLRLSCAASGFTFNTYAMNWVRQ | Humanized |
| | length sequence | APGKGLEWVARIRSKYNNYATYYADSVKDRFTISRDDSKSSL | |
| | | YLQMNNLKTEDTAMYYCVRHGNFGNSYVSWFAMWGQG | |
| | | TLVTVSS | |
| 29 | VH CDR3-Y114R | VRHGNFGNSYVSWFAR | Humanized |
| 30 | VH Y114R full | EVKLVESGGGLVQPGGSLRLSCAASGFTFNTYAMNWVRQ | Humanized |
| | length sequence | APGKGLEWVARIRSKYNNYATYYADSVKDRFTISRDDSKSSL | |
| | | YLQMNNLKTEDTAMYYCVRHGNFGNSYVSWFARWGQGT | |
| | | LVTVSS | |
| 31 | IgG1 constant | RTVAAPSVFIFPPSDEQLKSGTASVVCLLNNFYPREAKVQW | Human |
| | region LC Kappa | KVDNALQSGNSQESVTEQDSKDSTYSLSSTLTLSKADYEKHK | |
| | | VYACEVTHQGLSSPVTKSFNRGEC | |
| 32 | IgG1 constant | GQPKAAPSVTLFPPSSEELQANKATLVCLISDFYPGAVTVA | Human |
| | region LC | WKADSSPVKAGVETTTPSKQSNNKYAASSYLSLTPEQWKS | |
| | Lambda | HRSYSCQVTHEGSTVEKTVAPTECS | |
| 33 | IgG1-Fc | ASTKGPSVFPLAPSSKSTSGGTAALGCLVKDYFPEPVTVSW | Human |
| | | NSGALTSGVHTFPAVLQSSGLYSLSSVVTVPSSSLGTQTYICN | |
| | | VNHKPSNTKVDKRVEPKSCDKTHTCPPCPAPELLGGPSVFL | |
| | | FPPKPKDTLMISRTPEVTCVVVDVSHEDPEVKFNWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQVS | |
| | | LTCLVKGFYPSDIAVEWESNGQPENNYKTTPPVLDSDGSFFL | |
| | | YSKLTVDKSRWQQGNVFSCSVMHEALHNHYTQKSLSLSPG | |
| 34 | gp100₂₀₋₃₉ | AVGALKVPRNQDWLGVPRQL | Mouse |
| | peptide | | |
| 35 | H-2D^{b}-restricted | KVPRNQDWL | Mouse |
| | CD8⁺ T-cell | | |
| | epitope | | |
| 36 | OVA₂₄₁₋₂₇₀ | SMLVLLPDEVSGLEQLESIINFEKLTEWTS | Chicken |
| | peptide | | |
| 37 | chicken | SIINFEKL | Chicken |
| | ovalbumin | | |
| | OVA₂₅₇₋₂₆₄/H-2K^{b} | | |
| 38 | Mature murine | QDDAENIEYKVSISGTSVELTCPLDSDENLKWEKNGQELPQ | Mouse |
| | CD3ε (epsilon) | KHDKHLVLQDFSEVEDSGYYVCYTPASNKNTYLYLKARVCEY | (mature |
| | | CVEVDLTAVAIIIIVDICITLGLLMVIYYWSKNRKAKAKPVTRG | sequence) |
| | | TGAGSRPRGQNKERPPPVPNPDYEPIRKGQRDLYSGLNQR | |
| | | AV | |
| 39 | VH_IgG2amm | GFTFSGYG | Mouse |
| | Anti-CD3 145- | | |
| | 2C11_CDR1 | | |
| 40 | VH_IgG2amm | ITSSSINI | Mouse |
| | Anti-CD3 145- | | |
| | 2C11_CDR2 | | |
| 41 | VH_IgG2amm | ARFDWDKNY | Mouse |
| | Anti-CD3 145- | | |
| | 2C11_CDR3 | | |
| 42 | VH_IgG2amm | EVOLVESGGGLVQPGKSLKLSCEASGFTFSGYGMHWVRQA | Mouse |
| | Anti-CD3 145- | PGRGLESVAYITSSSINIKYADAVKGRFTVSRDNAKNLLFLQ | |
| | 2C11 | MNILKSEDTAMYYCARFDWDKNYWGQGTMVTVSS | |
| 43 | VL_IgG2amm | QDISNY | Mouse |
| | Anti-CD3 145- | | |
| | 2C11_CDR1 | | |
| | VL_IgG2amm | YTN | Mouse |
| | Anti-CD3 145- | | |
| | 2C11_CDR2 | | |
| 44 | VL_IgG2amm | QQYYNYPWT | Mouse |
| | Anti-CD3 145- | | |
| | 2C11_CDR3 | | |
| 45 | VL_IgG2amm | DIQMTQSPSSLPASLGDRVTINCQASQDISNYLNWYQQKP | Mouse |
| | Anti-CD3-145- | GKAPKLLIYYTNKLADGVPSRFSGSGSGRDSSFTISSLESEDIG | |
| | 2C11 | SYYCQQYYNYPWTFGPGTKLEIK | |
| 46 | VH_IgG2amm | GFNIKDYF | Mouse |
| | Anti-gp75 | | |
| | TA99_CDR1 | | |
| 47 | VH_IgG2amm | INPDNGNT | Mouse |
| | Anti-gp75 | | |
| | TA99_CDR2 | | |
| 48 | VH_IgG2amm | TRRDYTYEKAALDY | Mouse |
| | Anti-gp75 | | |
| | TA99_CDR3 | | |
| 49 | VH_IgG2amm | EVQLQQSGAELVRPGALVKLSCKTSGFNIKDYFLHWVRQRP | Mouse |
| | Anti-gp75 TA99 | DQGLEWIGWINPDNGNTVYDPKFQGTASLTADTSSNTVYL | |
| | | QLSGLTSEDTAVYFCTRRDYTYEKAALDYWGQGASVIVSS | |
| 50 | VL_IgG2amm | GNIYNY | Mouse |
| | Anti-gp75 | | |
| | TA99_CDR1 | | |
| | VL_IgG2amm | DAK | Mouse |
| | Anti-gp75 | | |
| | TA99_CDR2 | | |
| 51 | VL_IgG2amm | QHFWSLPFT | Mouse |
| | Anti-gp75 | | |
| | TA99_CDR3 | | |
| 52 | VL_IgG2amm | AIQMSQSPASLSASVGETVTITCRASGNIYNYLAWYQQKQG | Mouse |
| | Anti-gp75 TA99 | KSPHLLVYDAKTLADGVPSRFSGSGSGTQYSLKISSLQTEDS | |
| | | GNYYCQHFWSLPFTFGSGTKLEIK | |
| 53 | IgG2amm Fc | AKTTAPSVYPLAPVCGDTTGSSVTLGCLVKGYFPEPVTLTW | Mouse |
| | Anti-gp75 | NSGSLSSGVHTFPAVLQSDLYTLSSSVTVTSSTWPSQSITCN | |
| | CTA99-AAKR | VAHPASSTKVDKKIEPRGPTIKPCPPCKCPAPNAAGGPSVFI | |
| | | FPPKIKDVLMISLSPMVTCVVVDVSEDDPDVQISWFVNNVE | |
| | | VLTAQTQTHREDYNSTLRVVSALPIQHQDWMSGKEFKCKV | |
| | | NNKALPAPIERTISKPKGSVRAPQVYVLPPPEEEMTKKQVTL | |
| | | TCMVKDFMPEDIYVEWTNNGKTELNYKNTEPVLDSDGSYF | |
| | | MYSRLRVEKKNWVERNSYSCSVVHEGLHNHHTTKSFSRTP | |
| | | GK | |
| 54 | IgG2amm Fc | AKTTAPSVYPLAPVCGDTTGSSVTLGCLVKGYFPEPVTLTW | Mouse |
| | Anti-CD3-145- | NSGSLSSGVHTFPAVLQSDLYTLSSSVTVTSSTWPSQSITCN | |
| | 2C11-AALT | VAHPASSTKVDKKIEPRGPTIKPCPPCKCPAPNAAGGPSVFI | |
| | | FPPKIKDVLMISLSPMVTCVVVDVSEDDPDVQISWFVNNVE | |
| | | VLTAQTQTHREDYNSTLRVVSALPIQHQDWMSGKEFKCKV | |
| | | NNKALPAPIERTISKPKGSVRAPQVYVLPPPEEEMTKKQVTL | |
| | | TCMVTDFMPEDIYVEWTNNGKTELNYKNTEPVLDSDGSYL | |
| | | MYSKLTVEKKNWVERNSYSCSVVHEGLHNHHTTKSFSRTP | |
| | | GK | |
| 55 | Human | APTSSSTKKTQLQLEHLLLDLQMILNGINNYKNPKLTRMLTF | Human |
| | interleukin-2 | KFYMPKKATELKHLQCLEEELKPLEEVLNLAQSKNFHLRPRD | |
| | (UniProtKB - | LISNINVIVLELKGSETTFMCEYADETATIVEFLNRWITFCQSII | |
| | P60568) protein | STLT | |
| 56 | Human | NWVNVISDLKKIEDLIQSMHIDATLYTESDVHPSCKVTAMK | Human |
| | interleukin-15 | CFLLELQVISLESGDASIHDTVENLIILANNSLSSNGNVTESGC | |
| | (UniProtKB - | KECEELEEKNIKEFLQSFVHIVQMFINTS | |
| | P40933) protein | | |
| 57 | Rpl18₁₁₅₋₁₃₂ | KAGGKILTFDQLALESPK | Mouse |
| | peptide | | |
| 58 | LCMV SSP₂₇₋₄₈ | VITGIKAVYNFATCGIFALIS | LCMV |
| | peptide | | |

The present invention is further illustrated by the following examples, which should not be construed as being further limiting on the scope of the claims.

### EXAMPLES

### Example 1: Anti-Tumor Efficacy of CD3×TA99 Bispecific Antibody Treatment is Dependent Upon CXCR3-Mediated Infiltration of Immune Cells into the Tumor

Bispecific antibodies (bsAbs) targeting CD3 with one arm can redirect T cells to kill tumor cells by crosslinking CD3 on T cells with a tumor-associated antigen (TAA). Through the secretion of chemokines such as CXCL9, CXCL10, and CXCL11 from the tumor microenvironment, T cells and NK cell expressing the chemokine receptor CXCR3 can be attracted to the tumor. In Example, it was investigated how bsAb treatment is affected by the level of the immune infiltrate and the ability of immune cells, including T cells, to arrive at the tumor. To address this question, CXCR3 knockout (CXCR3-KO) mice were used, which exhibit a disruption of the CXCL9, CXCL10 and CXCL11-mediated cell trafficking that plays a non-redundant role in T cell infiltration into the tumor (Mikucki et al. Non-redundant requirement for CXCR3 signaling during tumoricidal T-cell trafficking across tumor vascular checkpoints. Nat Commun. 2015 Jun. 25; 6:7458).

All mouse strains were bred at the Leiden University Medical Centre (LUMC) animal facility, and all mouse experiments were performed at the animal facility of the LUMC, Leiden, The Netherlands. The health status of the animals was monitored over time and all animals tested negative for agents listed in the FELASA (Federation of European Laboratory Animal Science Associations) guidelines for specific-pathogen free mouse colonies. All mouse studies were approved by the Dutch animal ethics committee (CCD) and the local Animal Welfare Body of the LUMC on the permit number AVD11600202010004 (Examples 1, 10, and 11), AVD1160020188604 (Example 12), and AVD116002015271 (all other Examples). Experiments were performed in accordance with the Dutch Act on Animal Experimentation and EU Directive 2010/63/EU (‘On the protection of animals used for scientific purposes’). The B16F10 murine melanoma cell line (American Type Culture Collection [ATCC], cat. no. CRL-6475) was grown in Iscove's modified Dulbecco's medium (IMDM; Invitrogen, cat. no. 12440-053) supplemented with 8% fetal calf serum (FCS, Bodinco, cat. no. 5010 (Gibco, cat. no. 10378-016) at 37° C. in a humidified atmosphere containing 5% CO₂.

The CD3×TA99 bsAb (bsIGg2amm-2C11×TA99-LALA) with an inert Fc tail is based on the parental anti-CD3 145-2C11 and anti-glycoprotein (gp)75 TA99 antibodies, which recognize mouse-CD3ε and the TAA gp75, respectively. The amino acid sequences of the heavy (VH) and light chain (VL) variable regions of the CD3-binding arm are set forth in SEQ ID NOs:42 and 45, respectively. The amino acid sequences of the heavy and light chain variable regions of the gp75-binding arm are set forth in SEQ ID NOs: 49 and 52, respectively. For the CD3 binding arm, the VH CDR1, -2 and -3 sequences are provided in SEQ ID NOs: 39, 40, 41, respectively. The VL CDR1, -2 and -3 sequences are SEQ ID NO: 43, YTN and SEQ ID NO: 44, respectively. For the gp75-binding arm the VH CDR1, -2 and -3 sequences are provided in SEQ ID NOs: 46, 47, 48, respectively. The VL CDR1, -2 and -3 sequences are SEQ ID NO: 50, DAK and SEQ ID NO:51, respectively. The Fc tail was made inert with the Leu234Ala and Leu235Ala (LALA) mutations (Schlothauer et al., Protein Eng. Design and Selection 2016; 29(10):457-66). The bsAb was generated by introducing matching point mutations in the CH3 domains of the parental antibodies (Val370Lys and Lys409Arg in 2C11-145, Phe405Leu and Asn411Thr in TA99) that allowed controlled Fab-arm exchange. The bsAb was produced and purified in house as described previously (Labrijn et al., Efficient Generation of Bispecific Murine Antibodies for Pre-Clinical Investigations in Syngeneic Rodent Models, Sci Reports, 2017).

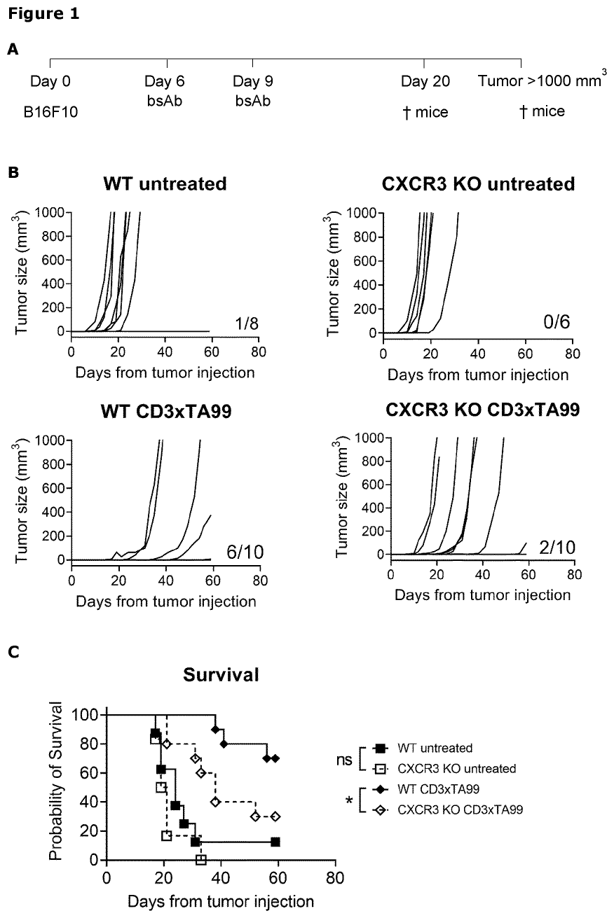

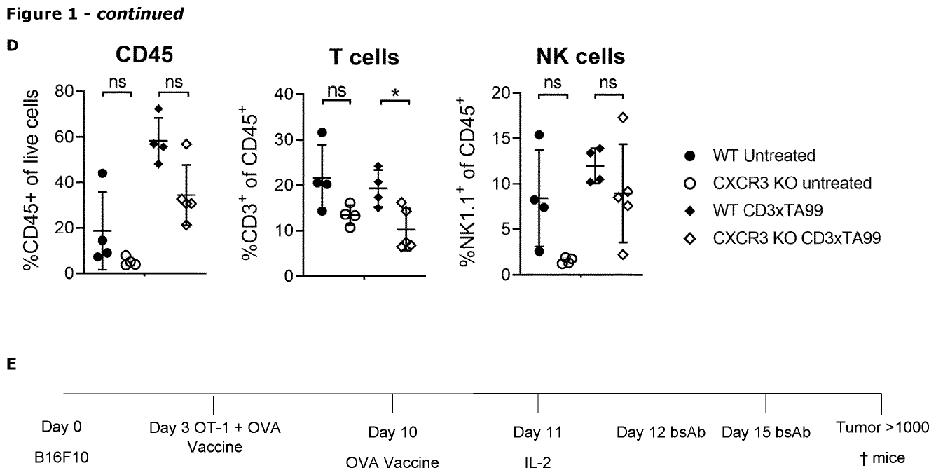

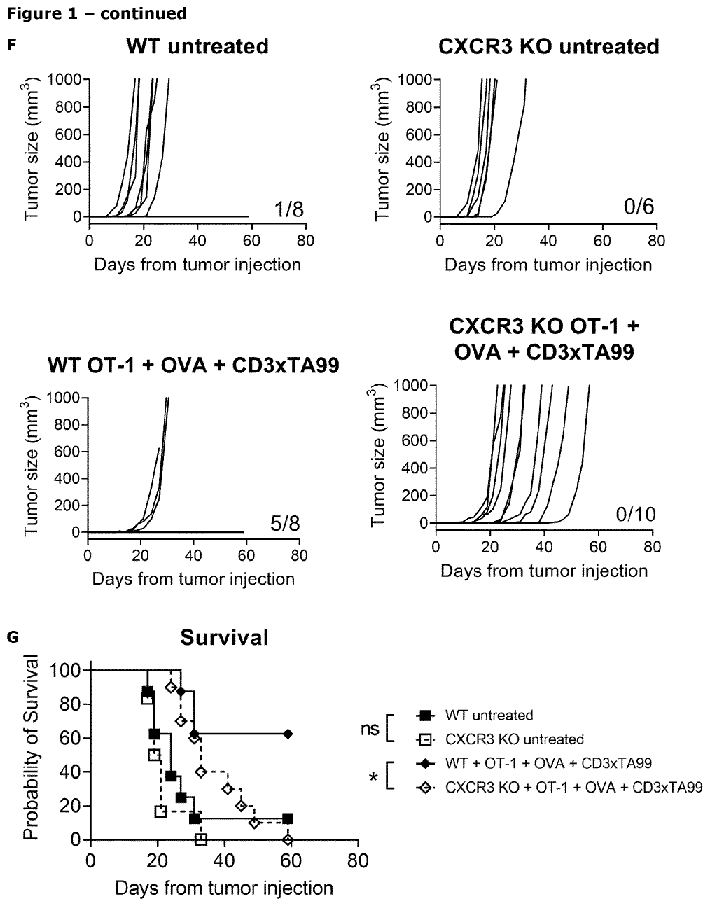

Wildtype (WT) male C57BL/6 mice (C57BL/6NCrl, Charles River, the Netherlands) and C57BL/6 CXCR3-KO mice (The Jackson Laboratory; stock no. 005796) were injected subcutaneously (s.c.) in the right flank with 50,000 syngeneic B16F10 melanoma cells (in 200 μL phosphate-buffered saline [PBS; Fresenius Kabi, cat. no. M090001] containing 0.1% bovine serum albumin [BSA; Sigma, cat. no. A3912]) at day 0. At day 6 and day 9, the mice were injected intraperitoneally (i.p.) with 12.5 μg (approximately 0.5 mg/kg) CD3×TA99 bsAb). A timeline of the treatment schedule is presented in FIG. 1A. Tumor sizes were measured three times weekly by caliper and calculated by multiplying length×width×height. Mice were euthanized when tumors reached a volume of 1000 mm³. A Mantel-Cox test was used to determine whether survival of the treated mice was significantly improved as compared to that of the untreated control group.

Both in untreated WT and CXCR3-KO mice, B16F10 tumors displayed rapid outgrowth with 24 and 20 days median survival, respectively, and 1 mouse remaining tumor-free in the WT group (FIG. 1B, C). In the CD3×TA99 bsAb-treated groups, survival of CXCR3-KO mice was significantly shorter compared to the WT group with fewer mice remaining tumor-free after 60 days (2/10 in CXCR3-KO group vs. 6/10 in WT group). CXCR3-KO mice, although not significant, showed a consistent trend of lower overall CD45-positive infiltrate, as well as lower frequencies of T and NK cells (FIG. 1D). This trend was maintained in mice treated with the CD3×TA99 bsAb, with significantly lower T-cell presence within the infiltrate.

Together, these data confirm the role of CXCR3-mediated immune cell trafficking in the efficacy of CD3-engaging bsAb therapy. This further suggests that modulating immune cell infiltration may contribute to improved outcome of the T-cell engaging bsAb treatment.

### Example 2: Tumor-Specific Vaccination in Combination with Transfer of Tumor-Specific T Cells Enhances the Efficacy of the T-Cell Engaging Bispecific Antibody CD3×TA99

In Example 1, it was shown that the recruitment of T cells to the tumor microenvironment impacts the anti-tumor efficacy of T-cell engaging bsAb treatment. Here, it was tested whether the anti-tumor efficacy of a T-cell engaging bsAb could be enhanced by administration of a tumor-specific vaccination in combination with tumor-specific T cells.

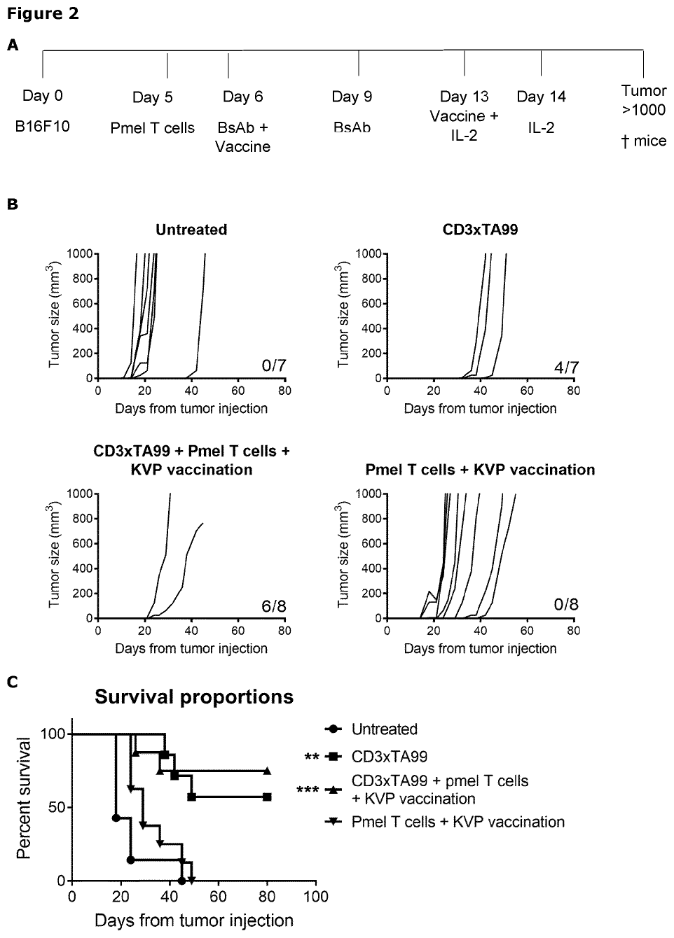

Tumor inoculation and bsAb treatment were performed essentially as described in Example 1, with the exception that here, 100,000 B16F10 cells were injected. A timeline of the treatment schedule is presented in FIG. 2A. For adoptive cell transfer (ACT), splenocytes with gp100₂₅₋₃₃/H-2D^{b} specific T-cell receptors (TCRs) were enriched from Pmel-1 TCR transgenic mice (Overwijk W W et al., Tumor regression and autoimmunity after reversal of a functionally tolerant state of self-reactive CD8⁺ T cells. J Exp Med 2003; 198:569-80; a kind gift of Dr. N. P. Restifo, Center for Cancer Research, National Cancer Institute, NIH, Bethesda, MD) that were bred to express the congenic markers CD45.1 or CD90.1. Lymphocytes from the spleen and lymph nodes of the Pmel-1 TCR transgenic mice were isolated and enriched for Pmel-1 T lymphocytes on nylon wool columns (Kisker Biotech GMBH, cat. no. MKN-50). These T lymphocytes will be further referred to as Pmel-1 T cells.

On day 6 and day 13, each mouse was anesthetized by an i.p. injection of a mixture of 20 μL of 20 mg/mL xylazine (Dechra, cat. no. 615319), 20 μL 10% ketamine (Alfasan, cat. no. 1907184-06) and 60 μL PBS, followed by immunization with a gp100-derived synthetic peptide. In short, the mice were injected s.c. on shaved left flanks with 100 μg of the gp100₂₀₋₃₉ peptide (‘KVP’) (AVGALKVPRNQDWLGVPRQL; SEQ ID NO: 34 homologous human sequence; synthesized in the Leiden University Medical Center, Leiden, The Netherlands, by Fmoc-based solid-phase peptide synthesis), which harbors a H-2D^{b}-restricted CD8⁺ T-cell epitope (\vec{KVP}RNQDWL; SEQ ID NO: 35), dissolved in 100 μl PBS. As adjuvant, 60 mg of 5% imiquimod-containing cream Aldara (3M Pharmaceuticals, cat. no. GTI102C) was simultaneously applied topically at the injection site. Recombinant human interleukin (IL)-2 (600,000 IU in 100 μL PBS, Proleukin®, Novartis, cat. no. 601381Z) was injected i.p. at the day of second immunization (day 13) and one day later (day 14). The term vaccination used in the Examples, such as KVP vaccination, refers to vaccination with a peptide vaccine, such as the KVP peptide, including Aldara adjuvant and IL-2 treatment, or including adjuvant CpG where indicated.

In untreated control mice, the syngeneic B16F10 tumor cells showed aggressive tumor growth, with a median survival of 18 days (FIG. 2B). Treatment with the bsAb CD3×TA99 significantly prolonged survival (FIG. 2C; Mantel-Cox test; P=0.0013), preventing tumor outgrowth in 4/7 mice. Combination of the CD3×TA99 treatment with KVP vaccination and ACT of Pmel-1 T cells further increased the anti-tumor efficacy, preventing tumor outgrowth in 6/8 mice. In contrast, ACT of Pmel-1 cells in combination with KVP vaccination alone was not sufficient to prevent tumor outgrowth and while the median survival of the mice (29 days) was slightly increased over that of the untreated controls, the treatment did not significantly improve survival (Mantel-Cox test; P=0.0755).

Taken together, these data show that tumor-specific vaccination in combination with ACT of tumor-specific T cells enhances the anti-tumor efficacy of CD3×TA99.

### Example 3: Anti-Tumor Efficacy of CD3×TA99 can be Enhanced by Tumor-Specific Vaccination Combined with ACT of Nonmatching, Tumor-Nonspecific T Cells, and by Tumor-Nonspecific Vaccination Combined with ACT of Matching, Tumor-Nonspecific T Cells

As shown in Example 2, the administration of the tumor-specific KVP vaccination in combination with 10 the ACT of matching, tumor-specific Pmel-1 T cells enhanced the anti-tumor efficacy of CD3×TA99 treatment in C57BL/6 mice bearing syngeneic B16F10 melanoma tumors.

Here, it was tested whether the anti-tumor efficacy of CD3×TA99 could also be enhanced by tumor-specific vaccination in combination with ACT of nonmatching, tumor-nonspecific T cells, or by tumor-nonspecific vaccination combined with ACT of matching, tumor-nonspecific T cells.

The experiments were performed as described in Example 1 and 2, with some minor adaptations as described below. Splenocytes with TCRs specific for chicken ovalbumin OVA₂₅₇₋₂₆₄/H-2K^{b} were enriched from OT1 TCR transgenic mice (The Jackson Laboratory, stock no. 003831) that were bred to express the congenic markers CD45.1 or CD90.1. Splenocyte enrichment was performed as described in Example 2 for the Pmel-1 T cells. These OVA-specific splenocytes will be further referred to as OT1 T cells.

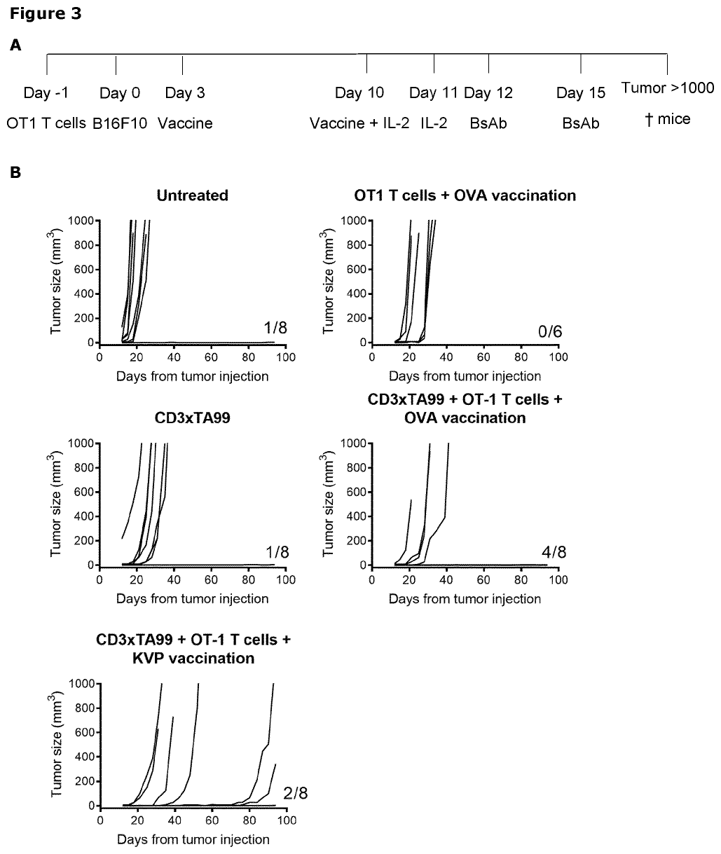

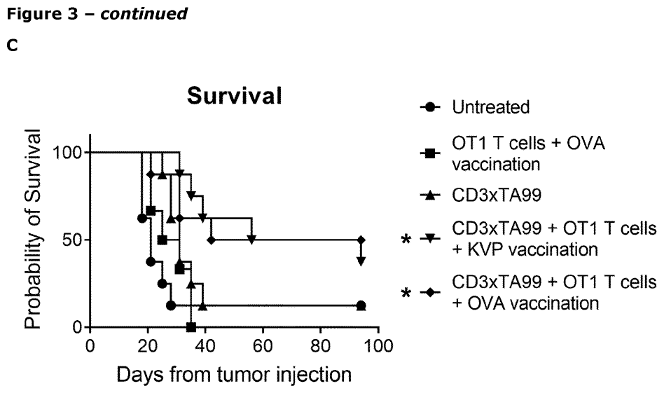

On day −1, male C57BL/6 mice were administered 1×10⁶ of the enriched OT1 T cells (in 200 μL PBS) via i.v. tail vein injection. On day 0, the mice were injected s.c. in the right flank with 100,000 syngeneic B16F10 melanoma cells (in 200 μL PBS with 0.1% BSA). On days 3 and 10, each mouse was immunized with 150 μg of the gp100₂₀₋₃₉ peptide (‘KVP peptide’) as described in Example 2, or with 150 μg of the chicken ovalbumin OVA₂₄₁₋₂₇₀ peptide (‘OVA peptide’) (SMLVLLPDEVSGLEQLESIINFEKLTEWTS; synthesized in the Leiden University Medical Center, Leiden, The Netherlands by Fmoc-based solid-phase peptide synthesis), which harbors a H-2K^{b}-restricted CD8⁺ T-cell epitope (SIINFEKL), dissolved in 100 μL PBS, after being anesthetized as described in Example 2. As adjuvants, Aldara was applied topically at the injection site on days 3 and 10, and IL-2 was injected i.p. on days 10 and 11. On days 12 and 15, the mice were injected i.p. with 12.5 μg CD3×TA99 bsAb. Tumor growth was monitored as described in Example 1. A timeline of the treatment schedule is presented in FIG. 3A.

In untreated control mice, the syngeneic B16F10 tumor cells showed aggressive tumor growth, with a median survival of 21 days (FIG. 3B). In 1 out of 8 mice, no tumor outgrowth was observed. Treatment with the bsAb CD3×TA99 did not significantly prolong survival (FIG. 3C; Mantel-Cox test; P=0.0972) but did extend the median survival to 31 days. This lack of efficacy is likely due to the delayed administration of the bsAb as compared to Example 2 (on day 12 and day 15 in Example 3, versus day 6 and day 9 in Example 2). Combination of the CD3×TA99 treatment with the tumor-nonspecific OVA vaccination and ACT of the matching, tumor-nonspecific OT1 T cells significantly increased the anti-tumor efficacy (*P=0.0147), with a median survival of 68 days. In addition, it prevented tumor outgrowth in 4 out of 8 mice. Similarly, combination of CD3×TA99 treatment with the tumor-specific KVP vaccination and ACT of the nonmatching, tumor-nonspecific OT1 T cells significantly delayed tumor outgrowth (*P=0.0147; median survival of 75 days), and prevented tumor outgrowth in 2 out of 8 mice. In contrast, treatment with only the tumor-nonspecific OVA vaccination in combination with the matching, tumor-nonspecific OT1 T cells did not significantly prolong survival (P=0.4; median survival of 28 days) or prevent tumor outgrowth.

Taken together, these data show that tumor-specific vaccination combined with ACT of nonmatching, tumor-nonspecific T cells, or tumor-nonspecific vaccination combined with ACT of matching, tumor-nonspecific T cells can both enhance the antitumor efficacy of CD3×TA99 to a similar extent as tumor-specific vaccination in combination with ACT of matching, tumor-specific T cells.

### Example 4: A Combination of CD3×TA99 bsAb and Tumor-Specific Vaccination or a Combination of CD3×TA99 bsAb and Tumor-Nonspecific Vaccination Combined with ACT of Matching, Tumor-Nonspecific T Cells Similarly Enhance Anti-Tumor Efficacy

As shown in Examples 2 and 3, the anti-tumor efficacy of bsAb CD3×TA99 can be enhanced by combining CD3×TA99 treatment with tumor-specific ACT and tumor-specific vaccination, by combining CD3×TA99 with tumor-non-specific ACT and a matching, tumor-non-specific vaccination, or by combining CD3×TA99 treatment with tumor-non-specific ACT and tumor-specific vaccination. Here, we studied whether the anti-tumor efficacy of CD3×TA99 could also be enhanced by combining CD3×TA99 treatment with tumor-specific vaccination in C57BL/6 mice bearing murine B16F10 melanoma tumors.

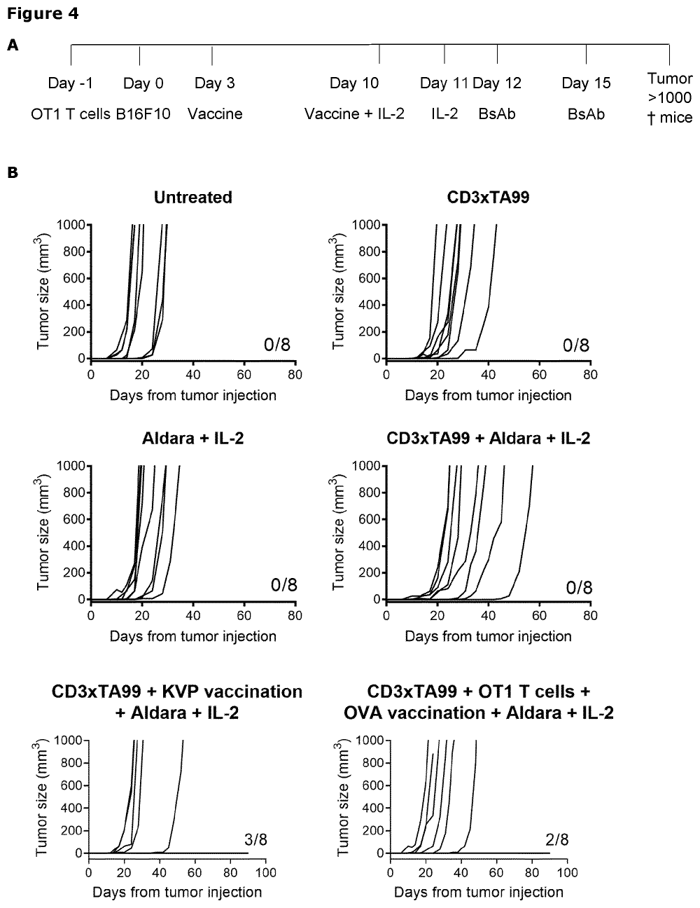

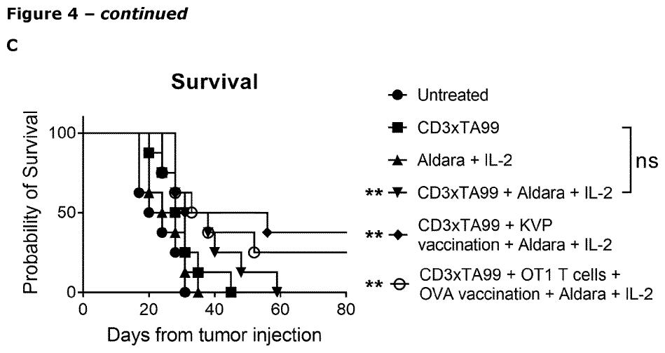

The experiments were essentially performed as described in Example 3. Tumor growth was monitored as described in Example 1. FIG. 4A shows the treatment timeline.

In untreated control mice, the syngeneic B16F10 tumor cells showed aggressive tumor growth, with a median survival of 22 days (FIG. 4B). No significantly prolonged survival was obtained by treatment with either the bsAb CD3×TA99 only or by treatment with Aldara and IL-2 only (FIG. 4C), however, the median survival was extended to 29.5 and 26 days, respectively. A significant delay in tumor outgrowth was observed after treatment with CD3×TA99, Aldara and IL-2 resulting in a significantly prolonged survival (P=0.0077), although all mice needed to be sacrificed due to tumor burden by day 59. The survival of the group of mice treated with CD3×TA99, Aldara and IL-2 was not significantly prolonged as compared to the groups of mice treated either with the CD3×TA99 bsAb only, or with Aldara and IL-2 only. When CD3×TA99 treatment was combined with tumor-non-specific (OVA) vaccination, tumor-non-specific ACT (OT1), 2 out of 8 mice survived and a significantly prolonged survival was observed as compared to the untreated group (P=0.0084). Similarly, 3 out of 8 mice survived the tumor challenge when treated with CD3×TA99 combined with tumor-specific (KVP) vaccination, Aldara and IL-2. This combination treatment significantly prolonged survival when compared to the untreated group (P=0.0092).

Together, these data show that the anti-tumor efficacy of CD3×TA99 can be enhanced by combining CD3×TA99 treatment with tumor-specific vaccination in the absence of ACT to the same extent as the combination of CD3×TA99 treatment with tumor-nonspecific ACT and matching, tumor non-specific vaccination. In contrast, addition of Aldara and IL-2 to CD3×TA99 treatment did not significantly increase survival.

### Example 5: T-Cell Infiltration and Activation is Enhanced in TRP1-Expressing Tumors Treated with CD3×TA99 bsAb in Combination with Tumor-Non-Specific ACT and Tumor-Nonspecific Vaccination

As shown in Examples 2, 3, and 4 the anti-tumor efficacy of the bsAb CD3×TA99 in the B16F10 murine tumor model can be enhanced by combining CD3×TA99 treatment with tumor-specific or tumor-non-specific ACT and vaccinations, as well as by combining CD3×TA99 treatment with tumor-specific vaccination. Here, we studied whether T-cell infiltration and activation is specifically enhanced in tumors expressing the TRP1 antigen (also known as gp75), which is recognized by the TA99 antibody, after treatment with CD3×TA99 in combination with tumor-non-specific ACT and vaccination.

To study T-cell infiltration and activation in vivo, OT1 CD8⁺ T cells derived from the TbiLuc×OT1 mouse were used (Kleinovink et al., Front. Immunol. 2018; 9:3097). T cells from the dual luciferase TbiLuc mouse constitutively express the green-emitting click-beetle luciferase (CBG99) and, upon T-cell activation, the red-emitting firefly luciferase (PpyRE9). The latter is induced by activation of Nuclear Factor of Activated T cells (NFAT) which is activated upon T-cell activation. This allows multicolor bioluminescence imaging of T-cell location and activation. The KPC3 tumor cell line was isolated from the genetic pancreatic ductal adenocarcinoma ‘KPC’ mouse model with K-ras^{G12D/+}, p53^{R172H/+} and pancreatic and duodenal homeobox 1 (Pdx-1)-Cre transgenes (Hingorani et al., Cancer Cell 2005; 7(5)469-3). KPC3-TRP1 cells were generated by transfection of a TRP1/gp75-coding plasmid using lipofectamine (Invitrogen), as previously described (Benonisson et al., Molec Canc Ther 2019; 18(2):312-22). This plasmid was kindly provided by Gestur Vidarsson (Academic Medical Center, Amsterdam, The Netherlands) and optimized by exchanging the zeocin selection gene with a neomycin selection gene and by replacing the cytomegalovirus (CMV) promoter with the CMV enhancer, chicken beta-actin promoter and rabbit beta-globin splice acceptor site (CAG) promoter. Transfected cells were selected with 400 μg neomycin for 7 days, after which they were enriched by fluorescence-activated cell sorting (FACS) on TRP1/gp75 expression using the TA99 antibody and a secondary Alexa Fluor-labeled anti-mouse IgG (Biolegend).

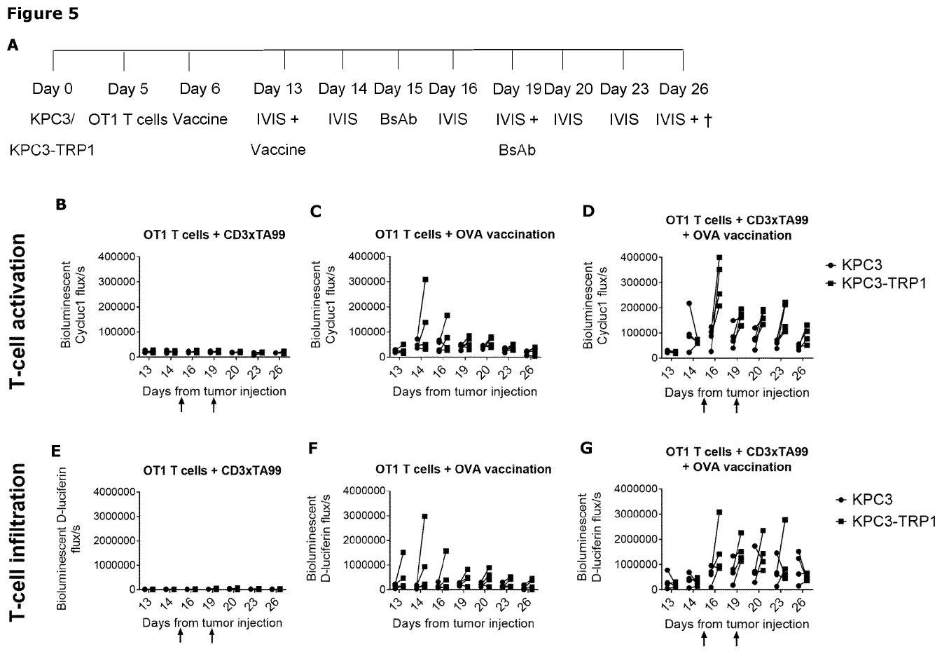

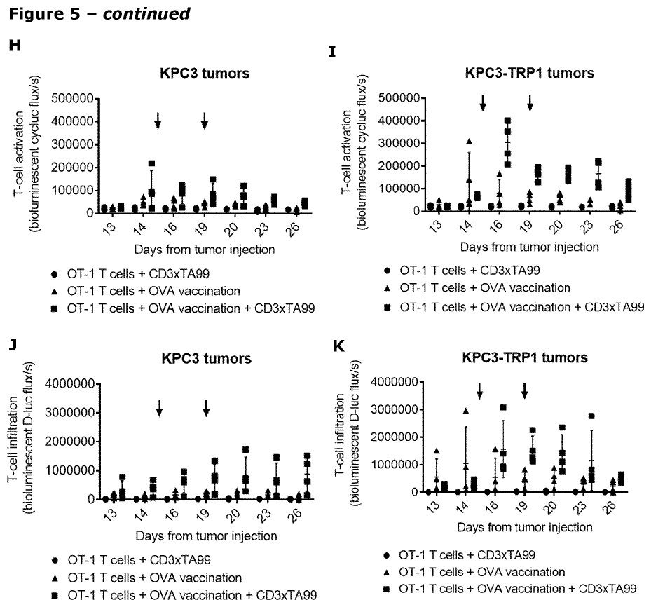

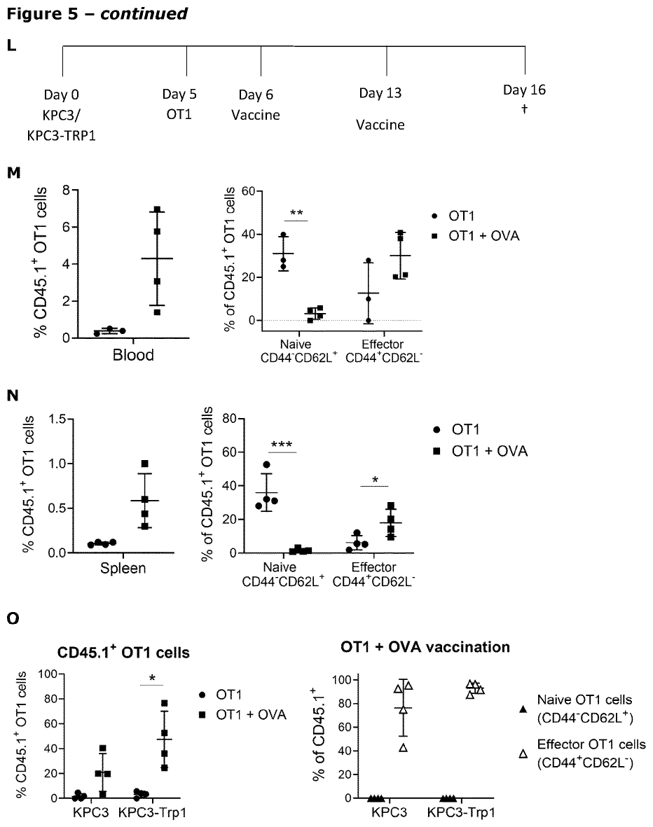

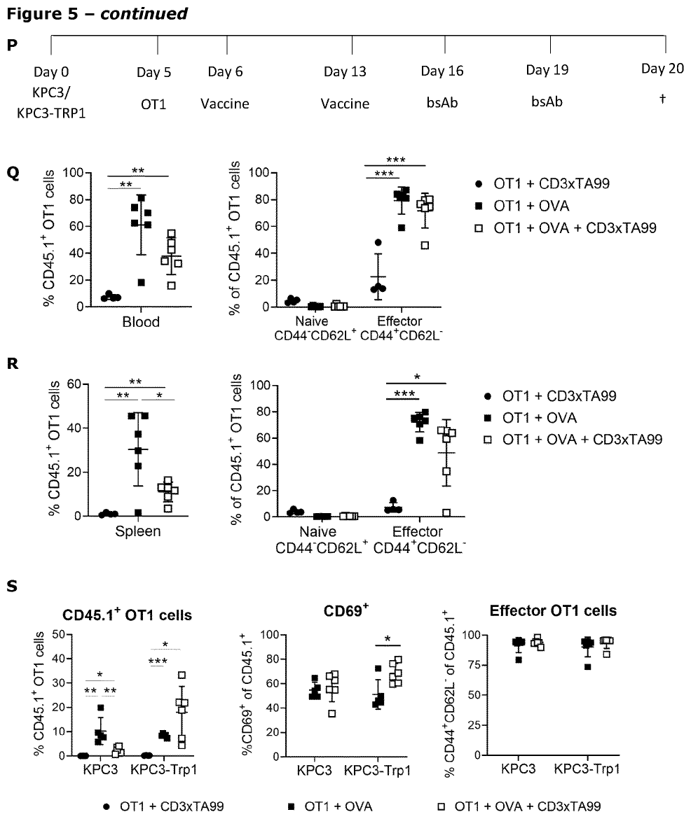

A timeline of the treatment schedule is presented in FIG. 5A. Albino C57BL/6 mice (Jackson Laboratories, stock number 000058) were shaved on two locations on the back and injected s.c. with 80,000 KPC3 tumor cells (left side of the back) and 80,000 tumor KPC3-TRP1 cells (right side of the back) on day 0. On day 5, mice received 1×10⁶ TbiLuc×OT1 enriched splenocytes (in 200 μL PBS) by i.v. injection into the tail vein. Next, randomized mice (n=4 per group) were treated with different combinations of OT1 T cell ACT, OVA peptide vaccination and CD3×TA99 antibody treatment as described below. Immunization with 100 μg OVA₂₄₁₋₂₇₀ synthetic long peptide mixed with 20 μg TLR9-ligand CpG (ODN-1826, cat no. tlrl-1826, InvivoGen) in 50 μL PBS was given via s.c. injection at the tail base on day 6 and 13. Mice received 12.5 μg CD3×TA99 (approximately 0.5 mg/kg) i.p. in 200 μL PBS on day 15 and 19. For bioluminescence visualization, mice were anesthetized via isoflurane inhalation (4% induction, 1.5% maintenance, Fendigo, cat. no. 1221894) and subsequently injected with 4.88 mg/kg Cycluc1 (Aobious, cat. no. A0B1117) in 100 μL PBS s.c. in the scruff of the neck. Mice were imaged using an IVIS Spectrum imager (Perkin Elmer, cat. no. 124262) on day 13 (just before the second immunization), day 14, day 16, day 19 (just before CD3×TA99 bsAb injection), day 23 and day 26. Bioluminescence was measured 15 minutes after the Cycluc1 injection using an open filter with an automatic exposure time. Next, mice were injected with 150 mg/kg D-Luciferin (Synchem, cat. no. bc219) in 100 μL PBS s.c. in the scruff of the neck, while remaining under isoflurane-induced anesthesia. Bioluminescence was measured after 15 minutes using a 540 nm filter and automatic exposure time settings. Signal quantification in specific regions of interest (ROIs) was performed by using fixed size ROIs throughout the experiment using LivingImage 4.2 software (PerkinElmer).

In both TRP1-positive and TRP1-negative KPC3 tumors, combined treatment with CD3×TA99 and OT1 T cells did not induce T-cell activation or infiltration (FIG. 5 B, E). Combined ACT of OT1 T cells and tumor-non-specific OVA vaccination resulted in T-cell activation and infiltration in a fraction of the mice bearing TRP1-positive KPC3 tumors, while a minor increase in T-cell infiltration and activation was observed in mice bearing the KPC3 tumors without TRP1-expression (FIG. 5 C, F). Upon adding the CD3×TA99 bsAb treatment to the OT1 T cell ACT and tumor-non-specific OVA vaccination, strong T-cell infiltration and activation of the OT1 T cells were observed in TRP1-positive tumors (FIG. 5 D, G). T-cell infiltration and activation were also to some extent observed in the TRP1-negative tumors (FIG. 5 H, J). In addition, T-cell activation in KPC3-TRP1 tumors was enhanced in mice receiving OT1 T cell ACT, OVA vaccination and the CD3×TA99 bsAb as compared to mice receiving OT1 T cells and the CD3×TA99 bsAb, and as compared to mice receiving OT1 T cell ACT and OVA vaccination (FIG. 5 I). Likewise, T-cell infiltration was enhanced in mice receiving OT1 T cell ACT, OVA vaccination and the CD3×TA99 bsAb as compared to mice receiving OT1 T cell ACT and the CD3×TA99 bsAb, and as compared to mice receiving OT1 T cell ACT and OVA vaccination (FIG. 5 K).

In addition to bioluminescence imaging, flow cytometry was used to confirm the effects of vaccination on OT1 T-cell activation and tumor infiltration. Timelines of the treatment schedules are presented in FIGS. 5L and 5P, which are in line with the treatment schedule described above. Randomized mice (n=4 per group) were treated with different combinations of OT1 T-cell ACT, OVA peptide vaccination and CD3×TA99 antibody treatment, as described above. Mice were euthanized on day 16 or on day 20, for time points at baseline and following bsAb administration, respectively. Single cell suspensions of the tumors were prepared by physical fragmentation followed by incubation with 300 μL of 385 μg/mL Liberase™ (Roche, cat. no. 05401020001) at 37° C. for 10 min in a humidified atmosphere containing 5% CO₂. Finally, cells were mashed through a 70 μm cell strainer (Falcon). Single cell suspensions of the spleens were prepared by physical fragmentation by mashing through a 70 μm cell strainer (Falcon) and subsequent treatment with lysis buffer (LUMC Pharmacy, NH4Cl 8.4 g/L and KHCO3 1 g/L, pH=7.4+/−0.2) to lyse red blood cells. Blood samples taken on day 16 and day 20 were treated with lysis buffer. Single cell suspensions were plated for flow cytometry staining in a 96-well plate (Greiner, cat. no. 650101).

Cells were resuspended in 40 μL Fc Block in PBS (1:400, BD, cat. no. 553141) and incubated 15 min on ice. Cells were washed 1× with PBS and resuspended in 40 μL ZombieAqua viability dye in PBS (1:800, Biolegend, cat. no. 423102) and incubated for 10 min at RT. Next, the cells were washed 1× with PBS and surface markers (CD45.1, CD45, CD8, CD44, CD62L, details in Table 6 were stained in 40 μL Brilliant Stain Buffer (BD, cat. no. 566349) for 20 min on ice. Samples were measured on a Fortessa cytometer (BD Biosciences) and analyzed with FlowJo software (Treestar).

TABLE 6
Fluorescently labeled antibodies used for flow cytometry

| | | | Catalog | Dilution |
|---|---|---|---|---|
| Antibody | Clone | Company | number | used |
| Anti-CD45- | 30-F11 | Biolegend | 103128 | 1:400 |
| AlexaFluor700 | | | | |
| Anti-CD45.1-APC | A20 | Biolegend | 110714 | 1:400 |
| Anti-CD8-PE | 53-6.7 | Biolegend | 100708 | 1:800 |
| Anti-CD69-FITC | H1.2F3 | Invitrogen | 11-0691-82 | 1:100 |
| Anti-CD44-BV785 | IM7 | Biolegend | 103059 | 1:100 |
| Anti-CD62L-BV421 | MEL-14 | Biolegend | 104436 | 1:100 |

On day 16 of the experiment, prior to bsAb administration, ACT of OT1 T cells alone did not induce T-cell activation or expansion in the blood (FIG. 5M), spleen (FIG. 5N), or both TRP1-positive and TRP1-negative KPC3 tumors (FIG. 5O). The combination of OT1 T-cell ACT and tumor-nonspecific OVA vaccination induced OT1 T-cell expansion in the blood and spleen (FIG. 5M, 5N), and significantly increased OT1 T cells in mice bearing TRP1-positive KPC3 tumors compared to ACT alone (FIG. 5O; * p<0.05). Although less pronounced and not significant, increases were also observed following combination treatment in the mice bearing KPC3 tumors without TRP1-expression. OT1 T cells switched from naïve to effector T (T_{eff}) cells following vaccination, with significantly higher levels of surface CD44 and reduced levels of CD62L (FIG. 5M-5O).

At a later timepoint (day 20; FIG. 5P), the combination of OT1 T-cell ACT and tumor-nonspecific OVA vaccination with or without the addition of CD3×TA99 bsAb resulted in a significant increase in peripheral OT1 T-cell numbers in the blood (FIG. 5Q; **, p<0.01) and spleen (FIG. 5R; **, p<0.01), compared to OT1 T-cell ACT alone. Following vaccination, the majority of OT1 T cells in blood (FIG. 5Q) and spleen (FIG. 5R) switched to effector T (T_{eff}) cells. The addition of CD3×TA99 bsAb treatment to the combination of OT1 T-cell ACT and OVA vaccination significantly decreased the percentage of OT1 T cells detected in spleen as compared to the group treated with OT1 T cell ACT and OVA vaccination only, while a similar trend was in blood (FIG. 5Q, 5R).

The combination of OT1 T-cell ACT and tumor-nonspecific OVA vaccination resulted in a significant increase in OT1 T cells in both the TRP1-positive and TRP1-negative tumors (FIG. 4S). When CD3×TA99 bsAb treatment was added to the OT1 T-cell ACT and tumor-nonspecific OVA vaccination combination, there was significantly enhanced T-cell infiltration and activation of the OT1 T cells in TRP1-positive tumors (FIG. 5S). These data are supported by the observed decrease in peripheral CD8⁺ OT1 T cells for the CD3×TA99 bsAb and vaccination combination (FIG. 5Q, 5R). In addition, the OT1 T cells in the TRP1-positive tumors after combination treatment comprising OT1 T-cell ACT, OVA vaccination and bsAb were more activated as mirrored by the significantly increased CD69 expression. OT1 T cells within tumors exhibited an effector T cell phenotype.

Together, these data show that OT1 T cells specifically infiltrate KPC3 tumors expressing the TRP1-antigen which is targeted by the CD3×TA99 bsAb, upon treatment with a combination of OT1 T-cell ACT, tumor-nonspecific OVA peptide vaccination and CD3×TA99 bsAb treatment. In addition, most of these infiltrating T cells become activated after the CD3×TA99 treatment. These data also show that the use of an alternative adjuvant (CpG) with the combination treatment also leads to tumor infiltration and activation of T cells.

### Example 6: Enhanced Tumor Infiltration of NK Cells, Macrophages and Dendritic Cells after Aldara and IL-2 Treatment of C57BL/6 Bearing a B16F10 Melanoma Tumor

In Examples 2-5, the anti-tumor efficacy of combination treatments comprising the CD3×TA99 antibody treatment, ACT and peptide vaccination, in combination with either Aldara (imiquimod) and IL-2, or CpG, was presented. The combination of the latter treatments resulted in enhanced anti-tumor efficacy in the B16F10 melanoma tumor model and an increase in T-cell infiltration and activation in KPC3 tumors expressing the TRP1-antigen which is targeted by the CD3×TA99 antibody. Here, we studied the effects of the adjuvants Aldara and IL-2 on the infiltration of different immune cell populations into the tumor microenvironment of B16F10 tumors in C57BL/6 mice.

C57BL/6 mice were injected s.c. with 100,000 B16F10 cells in 200 μL PBS supplemented with 0.1% BSA in the right flank. The mice were randomized and distributed over different treatment groups (n=4-6 mice per group). On day 7 and 14, 60 mg of 5% imiquimod-containing cream Aldara was topically applied, while recombinant human IL-2 was injected i.p. in 100 μL PBS on day 14 and 15. On day 16, all mice were euthanized. Single cell suspensions of the tumors were prepared by physical fragmentation followed by incubation with 300 μL of 385 μg/mL Liberase™ (Roche, cat. no. 05401020001) at 37° C. for 10 min in a humidified atmosphere containing 5% CO₂. Finally, cells were mashed through a 70 μm cell strainer (Falcon) and plated for flow cytometry staining in a 96-well plate (Greiner, cat. #650101).

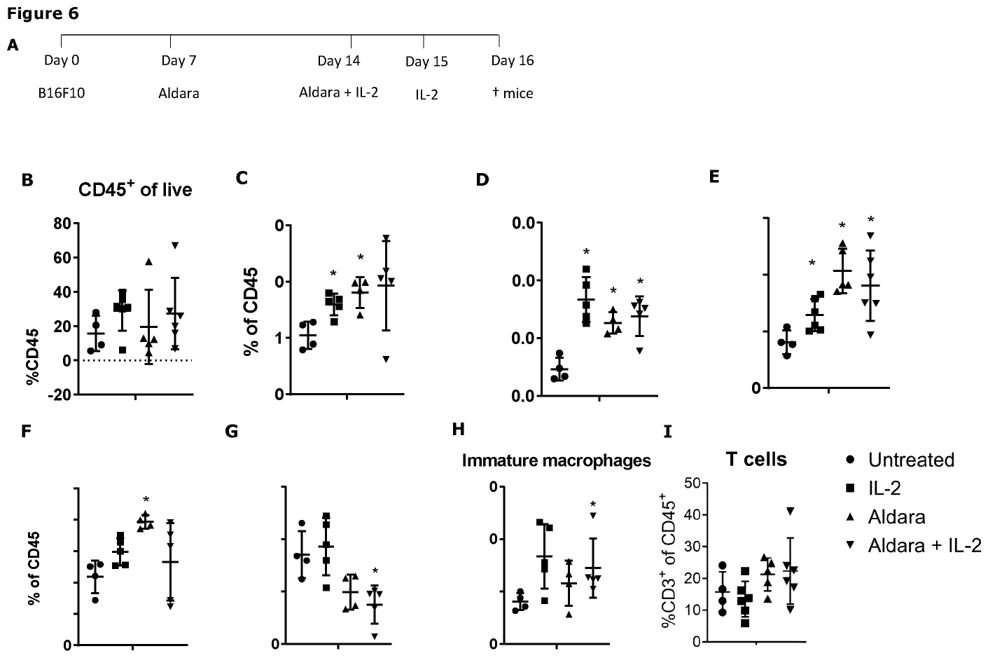

Cells were resuspended in 40 μL Fc Block in PBS (1:400, BD, cat. no. 553141) and incubated 10 mi on ice. Then, the cells were washed 1× with PBS and resuspended in 40 μL ZombieAqua viability dye in PBS (1:800, Biolegend, cat. no. 423102) and incubated for 10 min at RT. Next, the cells were washed 1× with PBS and surface markers (MHC-II, Ly6C, CCR2, Siglec-H, Siglec-F, Ly6G, CD103, F4/80, CD11b, CD45, CD11c, CD3, CD19, NK1.1, all in Table 6) were stained in 40 μL Brilliant Stain Buffer (BD, cat. no. 566349) for 20 m on ice. Intracellular Egr2 and iNOS staining (Table 7) was performed using the buffers provided with the FoxP3 staining kit (eBioscience, cat. no. 00-5523-00) according to the manufacturer's instructions. Samples were measured on a Fortessa cytometer (BD Biosciences) and analyzed with Flowlo software (Treestar). A timeline of the experiment is presented in FIG. 6A.

TABLE 7
Fluorescently labeled antibodies used for flow cytometry

| | | Catalog | Dilution |
|---|---|---|---|
| Antibody | Company | number | used |
| Anti-MHC-II-BV-421 | BD | 562564 | 1:400 |
| Anti-Ly6C-BV605 | Biolegend | 128036 | 1:500 |
| Anti-CCR2-FITC | Biolegend | 150608 | 1:200 |
| Anti-Siglec-H-BV650 | BD | 747672 | 1:300 |
| Anti-Siglec-F-BV711 | BD | 740764 | 1:200 |
| Anti-Ly6G-BV785 | Biolegend | 127645 | 1:400 |
| Anti-CD103-PerCP-Cy5.5 | Biolegend | 121416 | 1:100 |
| Anti-Egr2-PE | Invitrogen | 12-6691-82 | 1:100 |
| Anti-F4/80-PE-Cy5 | Biolegend | 123112 | 1:200 |
| Anti-CD11b-PE-Cy7 | Biolegend | 101216 | 1:1200 |
| Anti-iNOS-APC | eBioscience | 17-5920-80 | 1:100 |
| Anti-CD45-AlexaFluor700 | Biolegend | 103128 | 1:400 |
| Anti-CD11c-APC-Cy7 | Biolegend | 117324 | 1:100 |
| Anti-CD3-BV510 | Biolegend | 100353 | 1:800 |
| Anti-CD19-BV510 | BD | 562956 | 1:800 |
| Anti-NK1.1-BV510 | Biolegend | 108737 | 1:400 |

The mice were either left untreated (n=4), treated with IL-2 (n=5), Aldara (n=5), or with a combination of IL-2 and Aldara (n=6). No significant differences were observed between the treatment groups in the average percentages of immune cells (CD45⁺ cells) within the live cell population in tumors (FIG. 6B). Next, the percentages of tumor-infiltrating dendritic cell (DC) subsets, T cells, NK cells and macrophage subsets were analyzed within the CD45⁺ population (Table 8).

TABLE 8
Phenotypes of immune cell populations
analyzed using flow cytometry

| Population | Phenotype |
|---|---|
| cDC1 | CD11b⁻, CD11c⁺, MHCII⁺ |
| MoDC | CD11b⁺, CD11c⁺, MHCII⁺ and Ly6C⁺ |
| T cells | CD3⁺ |
| NK cells | NK1.1⁺, CD3⁻ |
| M1 macrophages | CD11b⁺, F4/80⁺, iNOS⁺ |
| M0 macrophages | CD11b⁺, F4/80⁺, iNOS⁻, Egr2⁻ |
| Immature macrophages | CD11b⁺, Ly6C⁺, MHCII⁺, CD11c⁻, CCR2⁻ |
| M2 macrophages | CD11b⁺, F4/80⁺, iNOS-Egr2+ |

Upon treatment of IL-2 or Aldara, significantly increased tumor infiltration by both conventional dendritic cells (cDC1) and monocyte-derived DCs (moDC) was observed (* p<0.05; FIG. 6C, D). Treatment with IL-2, Aldara, or the combination, resulted in a significant increase in tumor infiltration by NK cells as compared to untreated mice (FIG. 6E). A strong significant increase in tumor infiltration by M1 macrophages was observed after treatment with Aldara (FIG. 6F), but not with IL-2 or the combination of IL-2 and Aldara. The tumor infiltration by M0 macrophages, also known as resting macrophages, was decreased after the treatment with Aldara or the combination of Aldara and IL-2 (FIG. 6G). Despite a rather large spread within each group, the percentage of tumor-infiltrating immature macrophages was not significantly increased upon treatment with either IL-2 or Aldara, while the combination of IL-2 and Aldara did significantly increase the percentage of infiltrating immature macrophages (FIG. 6H; * p<0.05). No significant effect of Aldara, IL-2 or the combination of Aldara and IL-2 was detected on the infiltration of T cells (FIG. 6I).

Together, these data show that treatment with adjuvants Aldara and IL-2, either alone or in combination, induces enhanced B16F10 tumor infiltration by DC subsets, NK cells and macrophages. However, tumor-infiltration by macrophages varies per subset. The only subset that showed a decreased percentage within tumors of mice after treatment with Aldara and IL-2 was the subset of resting macrophages.

### Example 7: Intact Trafficking of Endogenous Cells Contributes to the Anti-Tumor Efficacy of a Combination Treatment Consisting of Antigen-Specific ACT, Cognate Antigen Vaccination and Bispecific Antibody Treatment

In Example 3 and 4, it was shown that a combination treatment consisting of tumor-nonspecific ACT, matching antigen vaccination and tumor-targeted bsAb treatment demonstrated anti-tumor efficacy in an in vivo tumor model. Here, it was investigated if the tumor control by adoptively transferred OT1 cells observed in Example 3 and 4 required a contribution of the endogenous compartment. To address this question, CXCR3-KO mice with disrupted endogenous T-cell trafficking were implanted with B16F10 tumors as outlined in Example 1. Mice received CXCR3-sufficient OT1 T cells combined with OVA vaccination (with Aldara and IL-2) on day 3, followed by OVA vaccination (with Aldara and IL-2) only on day 10. On days 12 and 15, mice were injected i.p. with CD3×TA99 treatment, essentially as described in Example 1. A timeline of the treatment schedule is presented in FIG. 1E.

No difference in tumor outgrowth was observed between WT and CXCR3-KO mice that were left untreated, as highlighted in Example 1 (FIG. 1F,G). The median survival of tumor-bearing CXCR3-KO mice treated with ACT of OT1 T cells coupled with OVA vaccination and CD3×TA99 bsAb treatment was prolonged to 33 days (FIG. 1F,G). Survival of WT mice receiving the same treatment was significantly prolonged (p=0.0171) with 5 out of 8 mice remaining tumor-free after 60 days.

These results indicate that the endogenous immune infiltrate contributes to the anti-tumor effect of a combination treatment consisting of tumor-nonspecific OVA vaccination and CD3×TA99 bsAb treatment when driven by exogenously transferred vaccine-specific OT1 T cells.

### Example 8: Combining CD3×TA99 bsAb Treatment and a Tumor-Specific Vaccination or Tumor-Nonspecific Vaccination Similarly Enhance Anti-Tumor Efficacy, without the Requirement of ACT

As shown in Example 4, the anti-tumor efficacy of CD3×TA99 could be enhanced by combining CD3×TA99 treatment with tumor-specific vaccination in the absence of ACT to the same extent as the combination of CD3×TA99 treatment with tumor-nonspecific ACT and matching, tumor non-specific vaccination. Here, we studied whether the anti-tumor efficacy of CD3×TA99 could also be enhanced by combining CD3×TA99 bsAb treatment with tumor-nonspecific vaccination in the absence of ACT in C57BL/6 mice bearing murine B16F10 melanoma tumors.

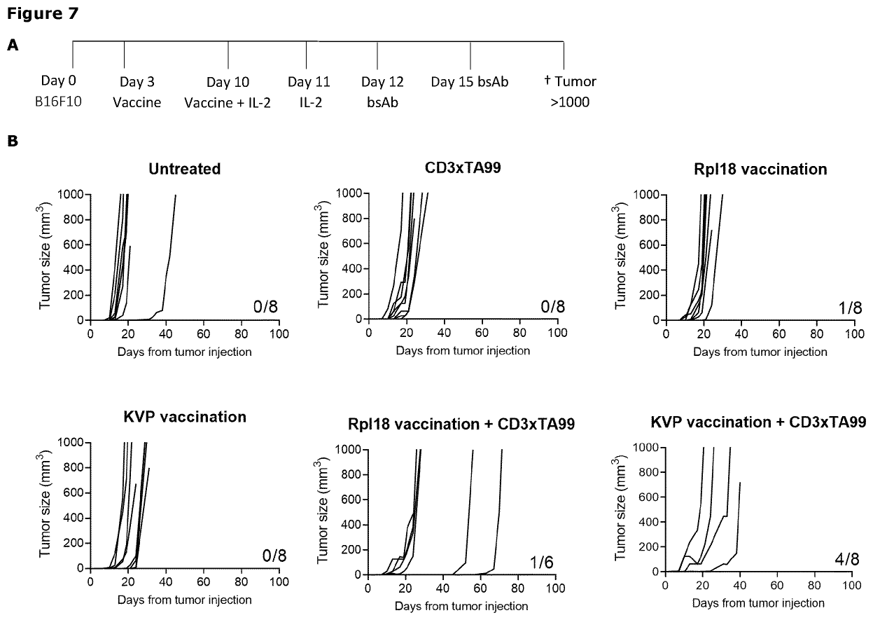

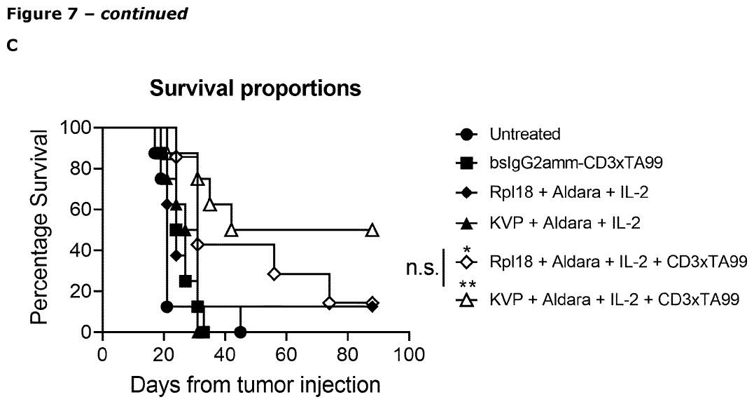

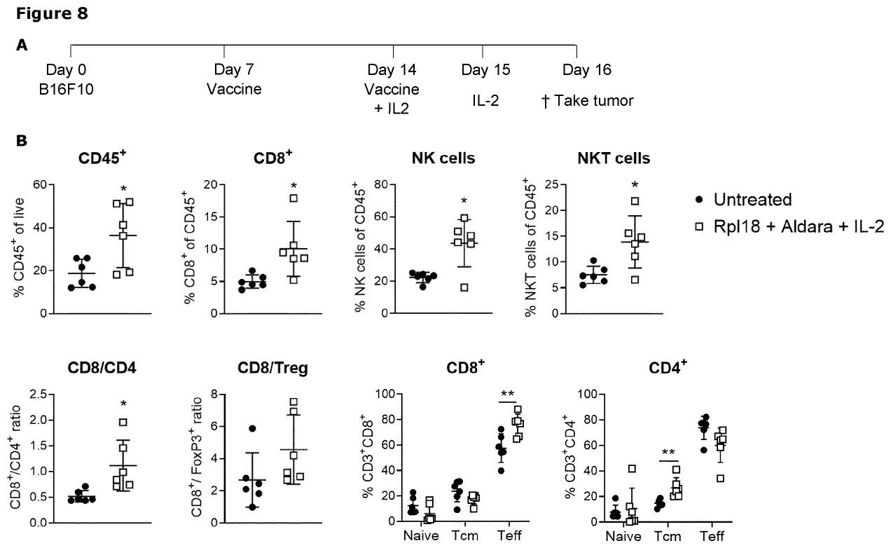

The experiments were essentially performed as described in Example 2 with the following exception: mice receiving a vaccination were injected s.c. in the tail base with 150 μg of either the tumor-specific KVP synthetic long peptide or a tumor-nonspecific synthetic long peptide derived from ribosomal protein L18 (Rpl18; SEQ ID NO: 57) in 100 μL PBS, and no ACT was given. Tumor growth was monitored as described in Example 1. FIG. 7A shows the treatment timeline.

In untreated control mice, the syngeneic B16F10 tumor cells showed aggressive tumor growth, with a median survival of 21 days (FIG. 7B). No significantly prolonged survival was obtained by treatment with CD3×TA99 bsAb only or with either Rpl18 or KVP vaccination, however, median survivals were extended to 25.5, 24 and 29 days, respectively. Significant delays in tumor outgrowth were observed when combining CD3×TA99 bsAb with either Rpl18 or KVP vaccination, resulting in a significantly prolonged survival when compared to CD3×TA99 bsAb treatment alone (P=0.0173 and P=0.0027, respectively; FIG. 7C). The median survival was extended to 31 days for the group of mice treated with CD3×TA99 bsAb and tumor-nonspecific Rpl18 vaccination, whereby 1 out of 6 mice survived the tumor challenge. When CD3×TA99 bsAb treatment was combined with tumor-specific KVP vaccination, the median survival was extended to 65 days and 4 out of 8 mice survived.

Together, these data show that the anti-tumor efficacy of CD3×TA99 bsAb can be enhanced by combining CD3×TA99 bsAb treatment with either a tumor-specific or tumor-nonspecific vaccination in the absence of ACT. In addition, despite the non-foreign nature of the Rpl18 peptide, an anti-tumor effect could be achieved when given in combination with appropriate adjuvants, which is supported by data from published reports showing that the addition of adjuvants to a vaccine antigen of non-foreign origin may overcome self-tolerance (Baumgaertner et al. Int J Cancer, 2011; 130(11): 2607-2617, Lienard et al. J Immunother. 2009; 32(8):875-83). Notably, a combination of CD3×TA99 treatment and adjuvants or the adjuvants only did not enhance survival as compared to CD3×TA99 treatment only as shown in Example 4, supporting the hypothesis that the anti-tumor effects observed in the current Example are dependent on addition of the vaccine antigen.

### Example 9: Tumor-Nonspecific Vaccination Increases Immune Cell Proportions and Activation within Tumors at the Time Point of bsAb Administration, Leading to Enhanced Survival when Combined with CD3×TA99 bsAb

In Example 8, the anti-tumor efficacy of CD3×TA99 bsAb and tumor non-specific vaccination combination treatment without the requirement of ACT was presented. In Example 6, it was shown that the adjuvants Aldara and/or IL-2 could induce increased frequencies of cDC1, MoDC, NK cell, M1 macrophage and immature macrophage populations within the tumor, and a decrease in the frequency of resting macrophages, while no significant changes in T cell frequencies were observed.

Here, we studied the effects of tumor-nonspecific vaccination on the infiltration of different immune cell populations into the tumor microenvironment of B16F10 tumors in C57BL/6 mice.

C57BL/6 mice were injected s.c. with 80,000 B16F10 cells in 200 μL PBS supplemented with 0.1% BSA in the right flank. The mice were randomized and distributed over different treatment groups (n=6 mice per group). On day 7 and 14, 60 mg of 5% imiquimod-containing cream Aldara was topically applied and 150 μg Rpl18 long peptide in 100 μL PBS was injected s.c. into the right flank. Recombinant human IL-2 was injected i.p. in 100 μL PBS on day 14 and 15. On day 16, all mice were euthanized. Single cell suspensions of the tumors were prepared as described in Example 5.

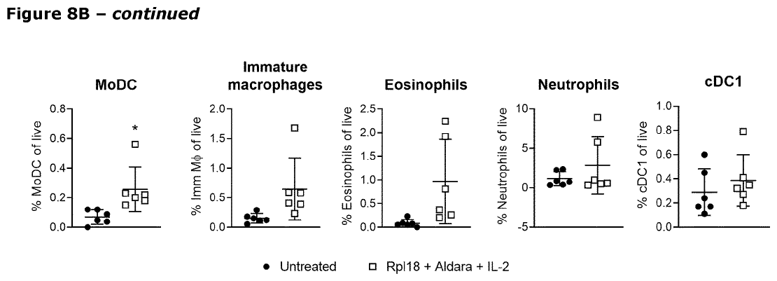

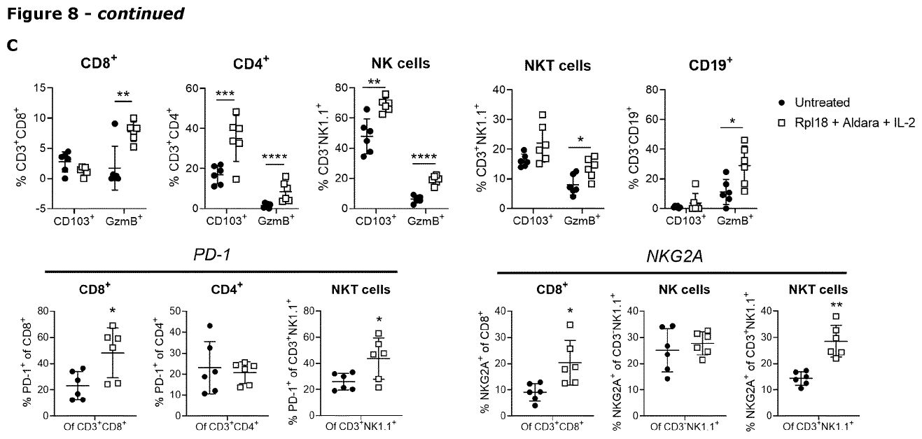

Cells were stained with fluorescently labeled antibodies directed against a variety of surface and intracellular markers using different flow cytometry staining panels (details in Tables 9 and 10), as described in Example 6. A timeline of the experiment is presented in FIG. 8A.

TABLE 9
Fluorescently labeled antibodies used
for Lymphoid Panel flow cytometry

| | | Catalog | |
|---|---|---|---|
| Antibody | Company | number | Dilution |
| Anti-CD62L-BV421 | Biolegend | 104436 | 1:100 |
| Anti-CD4-BV605 | Biolegend | 100547 | 1:1000 |
| Anti-NK1.1-BV650 | BD | 564143 | 1:100 |
| Anti-CD103-BV711 | BD | 121435 | 1:200 |
| Anti-CD44-BV785 | Biolegend | 103059 | 1:100 |
| Anti-CD19-FITC | Invitrogen | 11-0193-85 | 1:800 |
| Anti-CD8-PerCP-Cy5.5 | Biolegend | 100734 | 1:100 |
| Anti-FoxP3-PE | Invitrogen | 12-5773-82 | 1:100 |
| Anti-CD3-PE-CF594 | BD | 562286 | 1:400 |
| Anti-Granzyme B-PE-Cy7 | Invitrogen | 25-8898-82 | 1:200 |
| Anti-NKG2A-APC | Biolegend | 142808 | 1:100 |
| Anti-CD45-AlexaFluor700 | Biolegend | 103128 | 1:400 |
| Anti-PD1-APC-Cy7 | Biolegend | 135224 | 1:100 |

TABLE 10
Fluorescently labeled antibodies used
for Myeloid Panel flow cytometry

| | | Catalog | |
|---|---|---|---|
| Antibody | Company | number | Dilution |
| Anti-MHC-II-BV-421 | BD | 562564 | 1:400 |
| Anti-Ly6C-BV605 | Biolegend | 128036 | 1:500 |
| Anti-CCR2-FITC | Biolegend | 150608 | 1:200 |
| Anti-Siglec-H-BV650 | BD | 747672 | 1:300 |
| Anti-Siglec-F-BV711 | BD | 740764 | 1:200 |
| Anti-Ly6G-BV785 | Biolegend | 127645 | 1:400 |
| Anti-Egr2-PE | Invitrogen | 12-6691-82 | 1:100 |
| Anti-F4/80-PE-Cy5 | Biolegend | 123112 | 1:200 |
| Anti-CD11b-PE-Cy7 | Invitrogen | 25-0112-82 | 1:1200 |
| Anti-iNOS-APC | eBioscience | 17-5920-80 | 1:100 |
| Anti-CD45-AlexaFluor700 | Biolegend | 103128 | 1:400 |
| Anti-CD11c-APC-Cy7 | Biolegend | 117324 | 1:100 |
| Anti-CD8-PerCP-Cy5.5 | Biolegend | 100734 | 1:100 |
| Anti-CD3-BV510 (Dump) | BD | 563024 | 1:800 |
| Anti-CD19-BV510 (Dump) | BD | 562956 | 1:800 |
| Anti-NK1.1-BV510 (Dump) | Biolegend | 108737 | 1:400 |

The mice were either left untreated (n=6) or treated with a combination of Rpl18 vaccination with the adjuvants Aldara and IL-2 (n=6).

Immune cell subsets were characterized by the expression of the markers presented in Table 11. There was a significant increase in the percentage of immune cells (CD45⁺ cells) within the live cell population in tumor following tumor-nonspecific vaccination (FIG. 8B). Within the total immune cell population, CD8⁺ T cells, NK cells and NKT cell proportions were also significantly increased upon tumor-nonspecific vaccination (FIG. 8B; *, p<0.05). Similarly, vaccination led to an increased ratio of CD8⁺/CD4⁺ T cells and CD8⁺/Treg cells. CD8⁺ T effector and CD4⁺ T central memory cells were also significantly increased after vaccination (FIG. 8B; **, p<0.01).

In addition to increased proportions, the lymphoid population were also more activated (FIG. 8C). Granzyme B was significantly upregulated in CD8⁺ T cells (**, p<0.01), CD4⁺ T cells (****, p<0.0001), NK cells (****, p<0.0001), NKT cells (*, p<0.01) and CD19⁺ B cells (*, p<0.05) after vaccination. There were also significantly more CD103-expressing CD4⁺ T cells and NK cells in mice treated with the tumor-nonspecific vaccination, which is an indication of tissue residency of these cells in the tumor microenvironment (p<0.001 and p<0.01, respectively). In addition, PD-1 and NKG2A expression were significantly increased on CD8⁺ T cells (p<0.05) and NKT cells (p<0.05 and p<0.01).

TABLE 11
Phenotypes of immune cell populations
analyzed using flow cytometry

| Population | Phenotype |
|---|---|
| CD8⁺ T cells | CD3⁺, CD8⁺ |
| CD4⁺ T cells | CD3⁺, CD4⁺ |
| Treg | CD3⁺, CD4⁺, FoxP3⁺ |
| NKT cells | NK1.1⁺, CD3⁺ |
| MoDC | CD11b⁺, CD11c⁺, MHCII⁺ and Ly6C⁺ |
| NK cells | NK1.1⁺, CD3⁻ |
| M1 macrophages | CD11b⁺, F4/80⁺, iNOS⁺ |
| MO macrophages | CD11b⁺, F4/80⁺, iNOS⁻, Egr2⁻ |
| Immature macrophages | CD11b⁺, Ly6C⁺, MHCII⁺, CD11c⁻, CCR2 |
| CDC1 | CD11b⁻, CD11c⁺, MHCII⁺ |
| Eosinophils | CD11b⁺, Ly6G , SiglecF⁺ |
| Neutrophils | CD11b⁺, Ly6G⁺ |

Next, the percentages of myeloid cell subsets within the CD45⁺ population in the tumor were analyzed by flow cytometry. Upon treatment with tumor-nonspecific vaccination, increased expansion of monocyte-derived DCs (moDC; *, p<0.05) was observed in the tumor, while a trend for increased expansion was observed for immature macrophages, eosinophils and conventional dendritic cells (cDC1; FIG. 81B).

Together, these data show that treatment with tumor-nonspecific vaccination induced enhanced proportions of T cells, NK cells and DC subsets within B16F10 tumors preceding bispecific administration. In addition, immune cells subsets within the tumor were more activated after tumor-nonspecific vaccination, with elevated levels of granzyme B and activation markers. Comparing the results presented in Example 6 and Example 9, an increase in T-cell infiltration is only observed in Example 9 when mice were treated with a combination therapy comprising Rpl18 peptide vaccination and adjuvants Aldara and IL-2, as compared to mice treated with Aldara and IL-2 only (Example 6). This suggests that while an adjuvant may modulate the tumor microenvironment in a non-antigen specific manner, by inducing increased or decreased tumor infiltration by myeloid subsets and NK cells (as shown in Example 6), a vaccine antigen that triggers activation of T cells may be required for T-cell infiltration into the tumor.

### Example 10: Vaccination with a Tumor-Nonspecific Foreign Antigen Leads to Increased Infiltration, Activation and Effector Molecule Expression of CD8. T Cells Following T-Cell Engaging bsAb Treatment

Previous Examples have demonstrated improved tumor control following a treatment with CD3×TA99 bsAb together with a tumor-specific vaccination (KVP), tumor non-specific vaccination (Rpl18) or a combination of OT1 T cell ACT with a tumor-nonspecific foreign antigen vaccination (OVA). Furthermore, in Example 9, enhanced immune cell presence and activation was observed within tumors treated with Rpl18 peptide vaccination. Here, the contribution to CD3×TA99 bsAb treatment on immune cell presence and activation status within the tumor was studied of vaccination with a tumor-nonspecific foreign antigen (OVA).

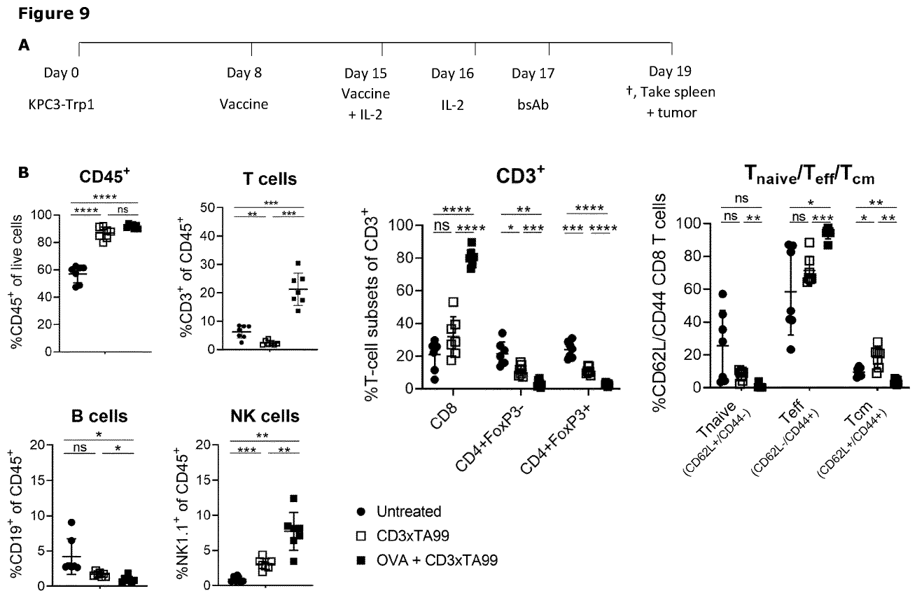

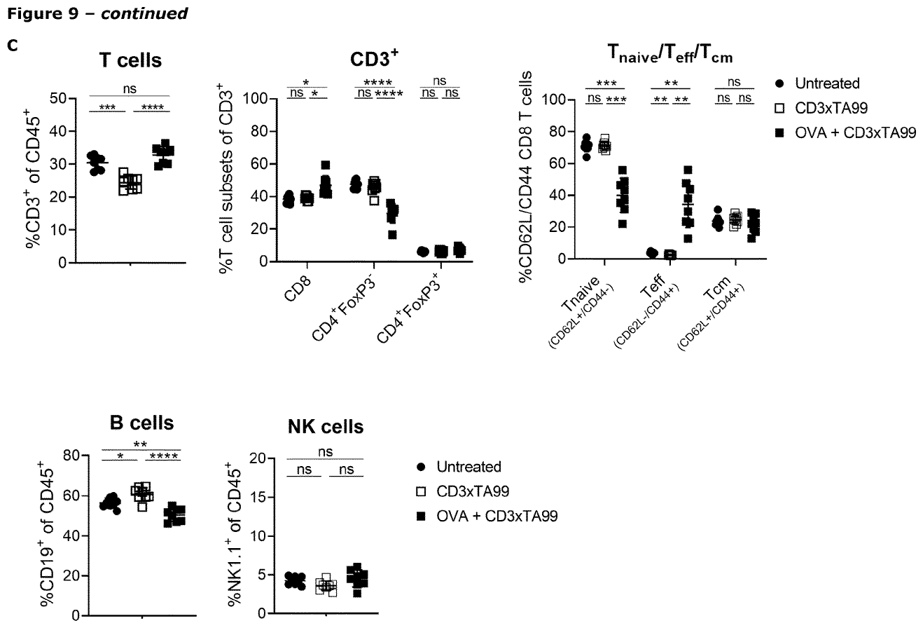

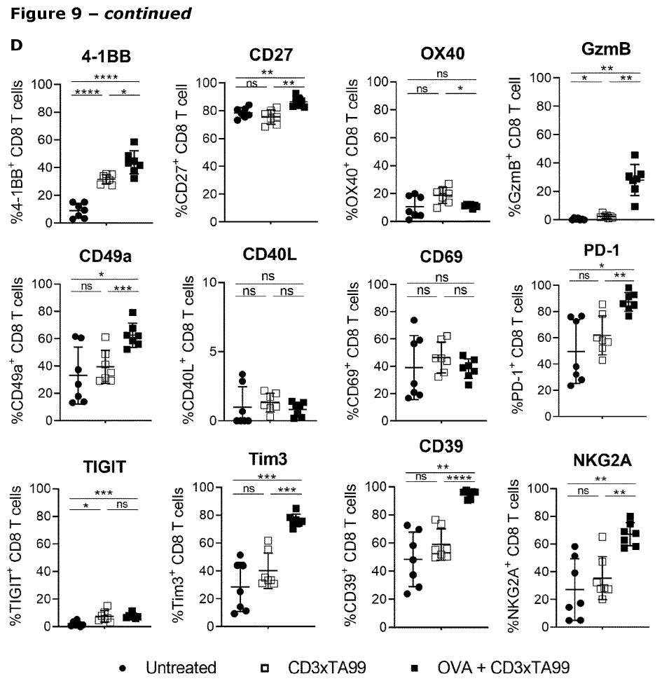

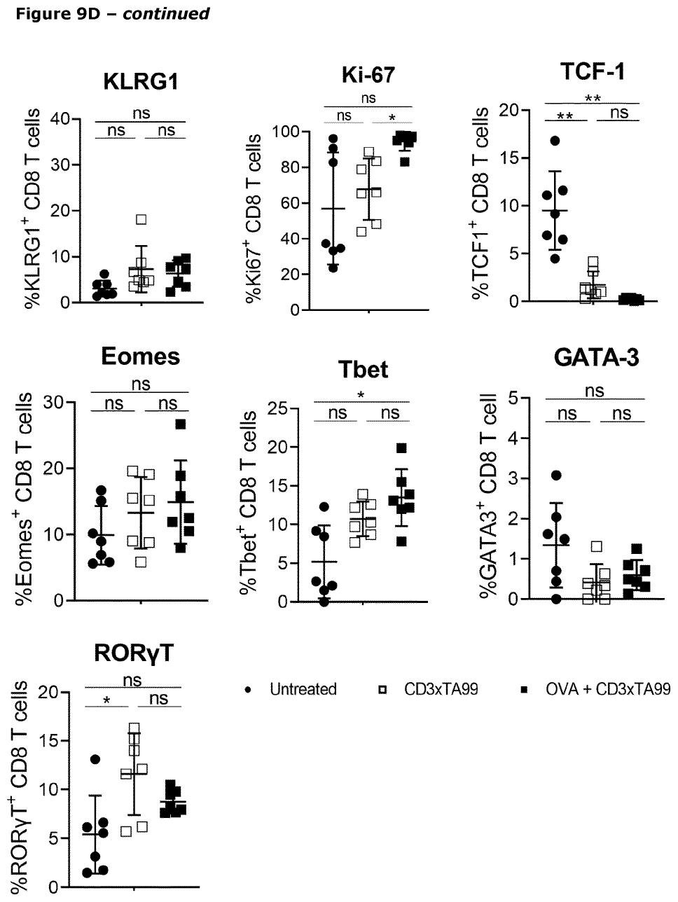

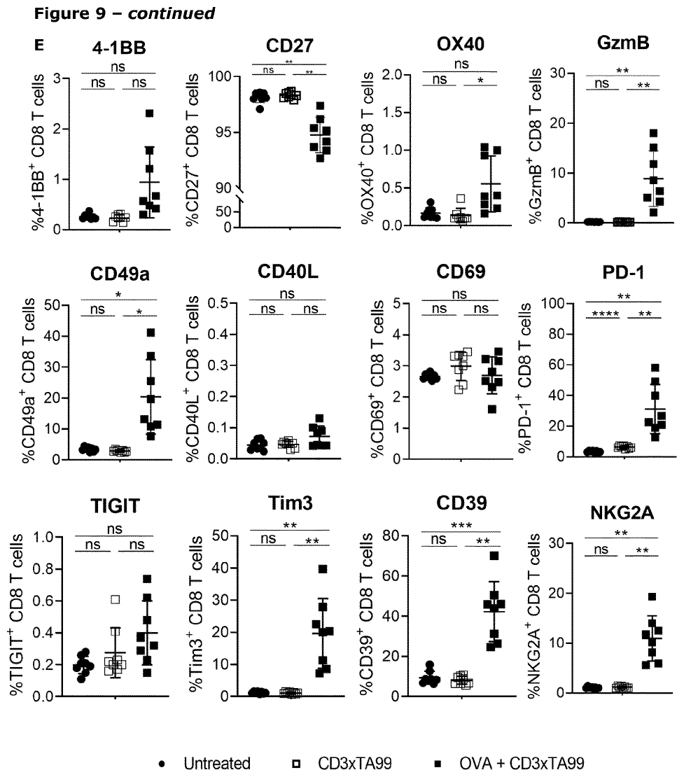

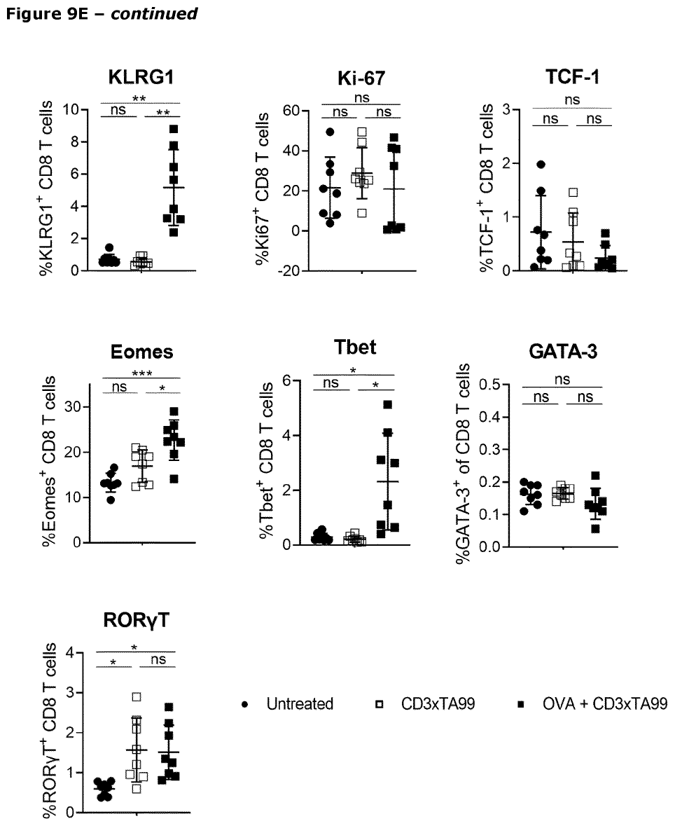

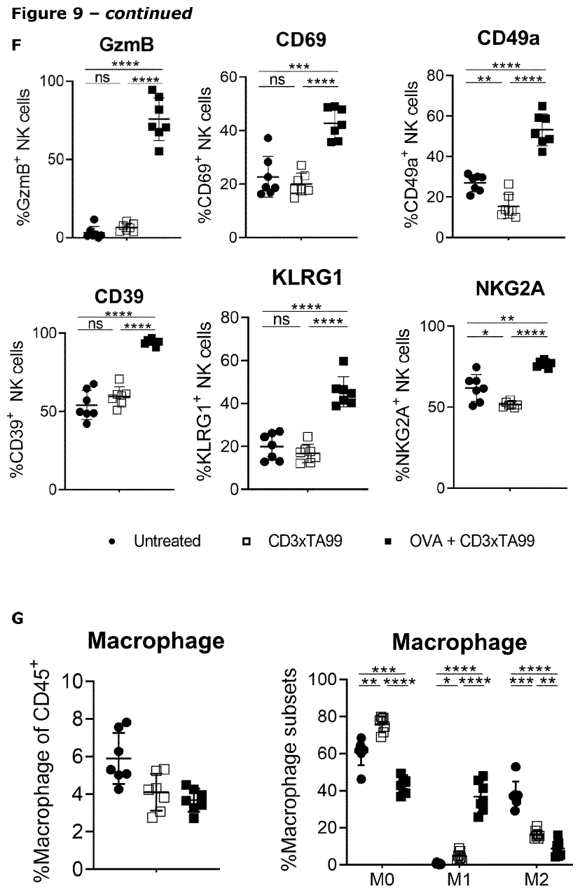

To address this, C57BL/6 mice were implanted with 80,000 TRP1-expressing KPC3 cells in 100 μL PBS supplemented with 0.1% BSA in the right flank on day 0 (FIG. 9A). The mice were randomized and distributed over different treatment groups (n=7 mice per group). On days 8 and 15, mice in the vaccine group were immunized with 150 μg OVA₂₄₁₂₇₀ peptide dissolved in 100 μL PBS, after being anesthetized as described in Example 2. As adjuvants, Aldara was applied topically at the injection site on days 8 and 15, and IL-2 was injected i.p. on days 15 and 16. On day 17, the mice were injected i.p. with 12.5 μg CD3×TA99 bsAb. Two days following bsAb treatment, all mice were sacrificed. Single cell suspensions of the tumors and spleens were prepared as described in Example 5. Staining was performed following the procedure outlined in Example 6 using antibody panels shown in Table 12, 13 and 14.

TABLE 12
Fluorescently labeled antibodies used for surface marker staining

| | | Catalog | |
|---|---|---|---|
| Antibody | Company | Number | Dilution |
| Anti-CD8-BUV395 | BD | 563786 | 1:400 |
| Anti-Viability-ZombieUV | BioLegend | 423107 | 1:400 |
| Anti-CD4-BUV496 | BD | 741051 | 1:600 |
| Anti-CD40L-BUV661 | BD | 741499 | 1:300 |
| Anti-CD49a-BUV737 | BD | 741776 | 1:200 |
| Anti-CD62L-BUV805 | BD | 741924 | 1:800 |
| Anti-CTLA-4-BV421 | Biolegend | 106312 | 1:200 |
| Anti-FoxP3-PacificBlue | Biolegend | 126410 | 1:200 |
| Anti-CD44-BV510 | Biolegend | 103043 | 1:600 |
| Anti-PD-1-BV605 | Biolegend | 135220 | 1:200 |
| Anti-NK1.1-BV650 | BD | 564143 | 1:200 |
| Anti-OX40-BV711 | Biolegend | 119421 | 1:200 |
| Anti-Tim-3-BV785 | Biolegend | 119725 | 1:300 |
| Anti-CD3-FITC | Invitrogen | 11-0031-85 | 1:800 |
| Anti-CD19-SparkBlue | Biolegend | 115566 | 1:800 |
| Anti-KLRG1-PerCP-Cy5.5 | Biolegend | 138418 | 1:200 |
| Anti-CD39-PE | Biolegend | 143804 | 1:200 |
| Anti-TIGIT-PE/dazzle | Biolegend | 142110 | 1:400 |
| Anti-CD122-PE-Cy5 | Biolegend | 123219 | 1:300 |
| Anti-NKG2A-PE-Cy7 | Biolegend | 142810 | 1:200 |
| Anti-4-1BB-APC | Biolegend | 106110 | 1:200 |
| Anti-CD45-AF700 | Biolegend | 103128 | 1:400 |
| Anti-Ly6G/CD11b/CD11c- | Biolegend | 127652 | 1:600 |
| APC/Fire750 (Dump) | Biolegend | 101262 | 1:600 |
| | Biolegend | 117352 | 1:400 |

TABLE 13
Fluorescently labeled antibodies used for intracellular
and transcription factor panel

| | | Catalog | |
|---|---|---|---|
| Antibody | Company | Number | Dilution |
| Anti-CD8-BUV395 | BD | 563786 | 1:400 |
| Anti-Viability-ZombieUV | Biolegend | 423107 | 1:400 |
| Anti-CD4-BUV496 | BD | 741051 | 1:800 |
| Anti-CD69-BUV737 | BD | 612793 | 1:200 |
| Anti-CD62L-BUV805 | BD | 741924 | 1:800 |
| Anti-CD25-BV421 | Biolegend | 102033 | 1:200 |
| Anti-FoxP3-PacificBlue | Biolegend | 126410 | 1:200 |
| Anti-CD44-BV510 | Biolegend | 103043 | 1:600 |
| Anti-Ki-67-BV605 | Biolegend | 652413 | 1:200 |
| Anti-NK1.1-BV650 | BD | 564143 | 1:200 |
| Anti-T-bet-BV711 | Biolegend | 644819 | 1:100 |
| Anti-CD27-BV785 | Biolegend | 124241 | 1:200 |
| Anti-GATA-3-AF488 | Biolegend | 653808 | 1:100 |
| Anti-CD19-SparkBlue | Biolegend | 115566 | 1:800 |
| Anti-Grz-B-PerCP-Cy5.5 | Biolegend | 372212 | 1:100 |
| Anti-ROR?T-PE | Invitrogen | 12-6988-82 | 1:100 |
| Anti-Eomes-PE/dazzle | Invitrogen | 61-4875-82 | 1:200 |
| Anti-CD3-PE-Cy5 | Biolegend | 100310 | 1:500 |
| Anti-CD28-PE-Cy7 | Biolegend | 102126 | 1:200 |
| Anti-TCF-1-APC | Cell Signaling Technology | 37636S | 1:100 |
| Anti-CD45-AF700 | Biolegend | 103128 | 1:400 |
| Anti-Ly6G/CD11b/CD11c- | Biolegend | 127652 | 1:600 |
| APC/Fire750 (Dump) | Biolegend | 101262 | 1:600 |
| | Biolegend | 117352 | 1:400 |

TABLE 14
Fluorescently labeled antibodies used
for Myeloid Panel flow cytometry

| | | Catalog | |
|---|---|---|---|
| Antibody | Company | Number | Dilution |
| Anti-CD40-BUV395 | BD | 745697 | 1:200 |
| Anti-Viability-Zombie UV | Biolegend | 423107 | 1:400 |
| Anti-CD86-BUV496 | BD | 750437 | 1:200 |
| Anti-CD11b-BUV563 | BD | 741242 | 1:1200 |
| Anti-CD70-BUV661 | BD | 741564 | 1:200 |
| Anti-PD-L1-BUV737 | BD | 741877 | 1:300 |
| Anti-CD62L-BUV805 | BD | 741924 | 1:500 |
| Anti-CD163-BV421 | Biolegend | 155309 | 1:200 |
| Anti-MHC-II-Pacific Blue | Biolegend | 107620 | 1:800 |
| Anti-CD3/CD19/NK1.1-BV510 | BD | 563024 | 1:800 |
| (Dump) | BD | 562956 | 1:800 |
| | Biolegend | 108737 | 1:800 |
| Anti-CD11c-BV605 | BD | 563057 | 1:200 |
| Anti-Siglec-H-BV650 | BD | 747672 | 1:200 |
| Anti-Siglec-F-BV711 | BD | 740764 | 1:400 |
| Anti-CCR2-BV785 | Biolegend | 150621 | 1:100 |
| Anti-iNOS-AF488 | Invitrogen | 53-5920-82 | 1:100 |
| Anti-Ly6G-Spark Blue | Biolegend | 127663 | 1:300 |
| Anti-Ly6C-PerCP-Cy5.5 | Biolegend | 128012 | 1:500 |
| Anti-XCR1-PE | Biolegend | 148204 | 1:100 |
| Anti-CD115-PE/dazzle | Biolegend | 135528 | 1:100 |
| Anti-F4/80-PE-Cy5 | Biolegend | 123112 | 1:300 |
| Anti-Arg1-PE-Cy7 | Invitrogen | 25-3697-82 | 1:200 |
| Anti-Egr2-APC | Invitrogen | 17-6691-82 | 1:100 |
| Anti-CD45-AF700 | Biolegend | 103128 | 1:400 |
| Anti-CD47-APC-Cy7 | Biolegend | 127526 | 1:200 |

The markers expressed by different immune cell populations are provided in Table 8 of Example 6. Treatment of mice with the CD3×TA99 bsAb resulted in an increased proportion of CD45⁺ cells within the tumor in both vaccinated and unvaccinated groups (FIG. 91B). In the vaccinated group, however, this was paralleled by a significant increase in T cell frequency, compared to the mice treated with CD3×TA99 only or left untreated. Furthermore, the combination treatment resulted in a skewing of the T-cell repertoire towards primarily CD8⁺ T cells, mirrored by the reduction in both conventional (FoxP3⁻) and regulatory (Foxp3⁺) CD4⁺ T cells. In addition, CD8⁺ T cells within the tumor switched almost exclusively to effector T (T_{eff}) cells when compared to the untreated group or the CD3×TA99 bsAb alone, in line with results described in Example 5 and 9. Combination treatment resulted in a significantly reduced relative frequency of B cells in the tumor, while the proportion of NK cells was significantly increased, as compared to the CD3×TA99 bsAb treatment only group. In the spleen, changes were less pronounced (FIG. 9C). Still, a minor shift was observed in T-cell composition towards CD8⁺ T cells, a reduction in naïve CD8⁺ T cells and a corresponding increase in CD8⁺ T_{eff} cells, suggestive of systemic activation within this group. Furthermore, B-cell frequency in the spleen was significantly reduced in the combination treatment group as compared to the CD3×TA99 bsAb treatment only group, while no effect was observed on NK cell frequency.

To understand how vaccination influences the T-cell infiltrate, a detailed phenotyping of CD8⁺ T cells within tumors and spleens was carried out (FIG. 9D, E). In the tumor, treatment with the CD3×TA99 bsAb alone resulted in a moderate increase in 4-1BB expression, as well as negative checkpoint TIGIT (FIG. 9D). The addition of vaccination, on the other hand, led to a further increase in cells positive for 4-1BB, CD27, CD49a and OX40. A third of cells in the combination group were positive for the effector molecule Granzyme B. Simultaneously, there was an increase in negative checkpoint expression, specifically, PD-1, Tim3 and NKG2A as well as the immunosuppressive ectonuclease CD39. Furthermore, CD8⁺ T cells from vaccinated mice were more proliferative, as measured by the expression of Ki-67. Transcription factor analysis revealed that both treatment groups downregulated TCF-1 that is normally associated with a naïve state, however the combination group showed a trend of greater skewing towards the T_{bet} positive CTL phenotype. Comparing these results with phenotyping of CD8⁺ T cells present in the spleen revealed that the changes caused by the bsAb alone were localized to the tumor (FIG. 9E). Conversely, the effects of combined treatment were more systemic.

In addition to changes in the T-cell compartment, an increase in NK cell frequency in the tumor was observed following combination therapy (FIG. 9B). Upon further inspection, a higher proportion of NK cells were activated and expressed more Granzyme B in the combination treatment group (FIG. 9F). Furthermore, a shift in the phenotype of tumor macrophages was observed, with a near complete loss of M2 macrophages, a reduction in M0 macrophages and a corresponding increase in M1 macrophages (FIG. 9G). Together, these observations suggest that the combination treatment effect is not limited to T cells but also results in a more favorable myeloid composition of the tumor microenvironment.

Overall, these data indicate that a combination of bsAb treatment and vaccination with a foreign tumor-nonspecific antigen leads to a greater CD8⁺ T cell and NK cell infiltrate that is more activated compared to the bsAb treatment alone.

### Example 11: Effect of Tumor-Nonspecific Foreign Antigen (OVA) Vaccination on the Anti-Tumor Efficacy of CD3×TA99 Bispecific Antibody Treatment

In Example 10, it was shown that the addition of a tumor-nonspecific foreign antigen vaccination to CD3×TA99 bsAb treatment resulted in a profound increase in infiltration and activation of endogenous T cells and NK cells. Therefore, it is studied here if this treatment regimen also yields enhanced control of tumor outgrowth, similar to immunization with a tumor-specific antigen (KVP) or tumor-nonspecific antigen (Rpl18), as shown in Example 8. Furthermore, an increase of the NK cell infiltrate and enhanced expression of Granzyme B following vaccination and CD3 bsAb treatment was observed in Example 10. Therefore, the contribution on NK cells to the combination treatment regimen is studied by depleting them prior to vaccination.

To address this, C57BL/6 mice are implanted with 50,000 B16F10 tumor cells in 100 μL PBS supplemented with 0.1% BSA in the right flank on day 0. The mice are randomized and distributed over different treatment groups (n=8-12 mice per group). To investigate the role of NK cells, 100 μg depleting anti-NK1.1 antibody (Invivoplus anti-NK1.1, BioXcell BP0036) is injected i.p 2 days prior to tumor injection and then on days 2, 9 and 16. On days 3 and 10, mice in the vaccine groups are immunized with 150 μg OVA₂₄₁₂₇₀ peptide, dissolved in 100 μL PBS, after being anesthetized as described in Example 2. As adjuvants, Aldara is applied topically at the injection site on days 3 and 10, and recombinant human IL-2 is injected i.p. on days 10 and 11. On day 12 and 15, the mice are injected i.p. with 12.5 μg CD3×TA99 bsAb. Tumor outgrowth is monitored by measuring tumors twice weekly and mice are sacrificed when tumor volumes exceed 1000 mm³.

### Example 12: Enhanced Anti-Tumor Efficacy by a Combination Treatment Comprised of CD3×TA99 bsAb and a Prime-Boost Influenza Vaccination Regimen in a Murine Tumor Model

In Example 8, it was shown that the anti-tumor efficacy in a murine tumor model could be enhanced by a combination treatment comprising a tumor-specific bsAb and a tumor-nonspecific vaccination without the requirement of ACT. Here, it was studied whether anti-tumor efficacy could also be enhanced by combining bsAb treatment alongside a vaccine regime to boost pre-existing virus-specific T cells in a murine tumor model.

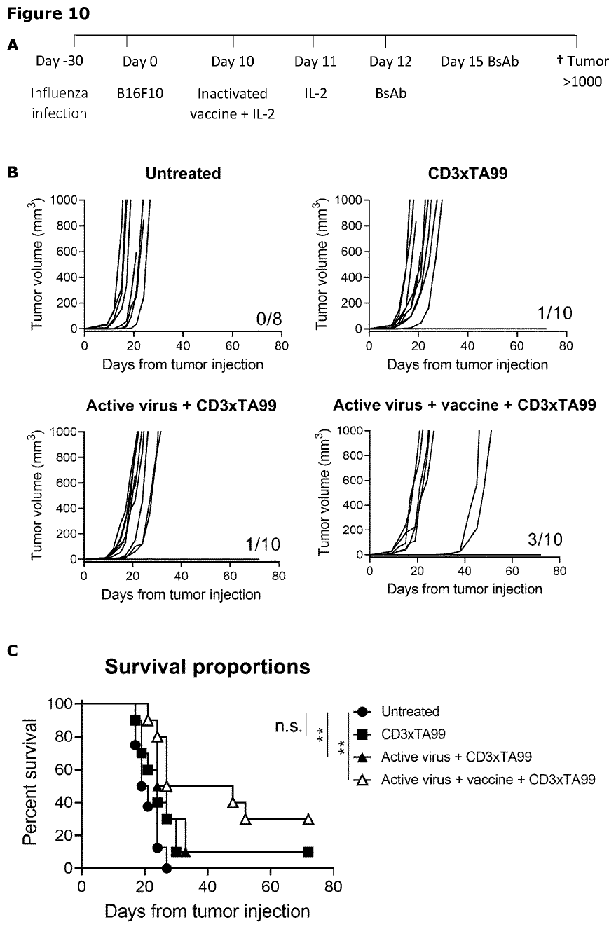

C57BL/6 mice were administered with a low dose influenza virus 100×TCID50 (HK×31; Sanquin, The Netherlands) intranasally on day −30 of the study. Following complete resolution of active infection, the mice were injected s.c. with 80,000 B16F10 cells in 200 μL PBS supplemented with 0.1% BSA in the right flank on day 0. The mice were randomized and distributed over different treatment groups (n=10 mice per group). On day 10, 60 mg of 5% imiquimod-containing cream Aldara was topically applied and 2×10⁶ TCID50 heat-inactivated influenza virus (1 h at 70° C.) was injected s.c. into the contralateral flank. Recombinant human IL-2 was injected i.p. in 100 μL PBS on day 10 and day 11. Mice received 12.5 μg CD3×TA99 (approximately 0.5 mg/kg) i.p. in 200 μL PBS on day 12 and 15. Tumor growth was monitored as described in Example 1. A timeline of the treatment schedule is presented in FIG. 10A.

In untreated control mice, the syngeneic B16F10 tumor cells showed aggressive tumor growth, with a median survival of 20 days (FIG. 10B). No significantly prolonged survival was obtained by treatment with bsAb CD3×TA99 only, however, median survival was extended to 24 days (FIG. 10B). Significant delays in tumor outgrowth were observed when administering CD3×TA99 bsAb after active influenza infection or after the combination of active infection and vaccination, resulting in a significantly prolonged survival when compared to untreated mice (P=0.0096 and P=0.0011, respectively; FIG. 10C). The median survival was extended to 25.5 days for the group of mice treated with CD3×TA99 bsAb following active influenza infection, whereby 1 out of 10 mice survived the tumor challenge. When CD3×TA99 bsAb treatment was combined with active infection and vaccination, the median survival was extended to 37.5 days and 3 out of 10 mice survived.

Together, these data show that the anti-tumor efficacy of CD3×TA99 bsAb treatment can be enhanced by combining CD3×TA99 bsAb treatment with boosted pre-existing virus-specific T cells in a murine tumor model.

### Example 13: Enhanced Anti-Tumor Efficacy by a Combination of Boosted Pre-Existing Tumor-Nonspecific LCMV-Specific T Cells and CD3×TA99 bsAb Treatment in a Murine Tumor Model

In the previous Examples, it was demonstrated that the anti-tumor efficacy of a CD3-engaging bsAb could be enhanced by combining it with a tumor-nonspecific vaccine. In Example 12, a combination treatment comprising a CD3-engaging bsAb and an Influenza vaccine, preceded by active Influenza infection, was shown to enhance anti-tumor efficacy. Here, it is studied whether the anti-tumor efficacy of a CD3-engaging bsAb can also be enhanced by combining CD3-engaging bsAb treatment with lymphocytic choriomeningitis virus (LCMV)-specific vaccination in mice that have been previously infected with LCMV.

To study this, C57BL/6 mice are administered with 2×10⁵ PFU of LCMV-Armstrong (LUMC, the Netherlands) i.p. in 100 μl PBS on day −30 of the study. LCMV expresses the non-self-antigens gp33 and gp34. Following complete resolution of active infection, the mice are injected s.c. with 50,000 B16F10 cells in 100 μL PBS supplemented with 0.1% BSA in the right flank on day 0. The mice are randomized and distributed over different treatment groups (n=8 mice in the control group; n=12 in treatment groups). On day 8, 100 μg of SSP₂₇₋₄₈ synthetic long peptide containing gp33 and gp34 epitopes (synthesized in-house, LUMC, The Netherlands; SEQ ID NO: 58) together with 20 μg CpG-ODN1826 dissolved in PBS is injected s.c. in the tailbase of the mice. Mice receive 12.5 μg CD3×TA99 (approximately 0.5 mg/kg) i.p. in 200 μL PBS on day 12 and 15. Tumor growth is monitored as described in Example 1.

### CONCLUDING REMARKS ON EXAMPLES

The data presented in the Examples above demonstrate various proposed regimens to enhance the anti-tumor efficacy of T-cell engaging bsAbs. Firstly, it was shown that adoptively transferred tumor-specific or tumor-nonspecific T cells may constitute an additional source of T cells to enable T-cell engaging bsAbs to exert their anti-tumor effect, when given in combination with a cognate vaccine. Secondly, it was shown that the anti-tumor efficacy of a T-cell engaging bsAb could also be enhanced by a combination of a tumor-specific or tumor-nonspecific vaccine and bsAb treatment in the absence of adoptively transferred T cells. Furthermore, it was shown that the effect of adjuvants that are co-formulated in such vaccine compositions may help modulate the suppressive tumor microenvironment, by driving increased frequencies of non-suppressive leukocyte populations such as antigen-presenting cells and NK cells. A combination treatment comprising a vaccine antigen and adjuvants was shown to induce increased frequencies of T cells and NK cells, that exhibit more activated phenotypes, in the tumor prior to the proposed timing of bsAb treatment. Lastly, a combination treatment comprising a T-cell engaging bsAb targeting a tumor-specific antigen and a tumor-nonspecific immunogenic composition, which is able to boost pre-existing T cells, was shown to induce significantly increased frequencies of tumor-infiltrating T cells with a predominantly activated Teff phenotype.

A generalized concept is shown for enhancing anti-tumor efficacy of T-cell engaging bsAbs by combining such bsAbs with a vaccination regimen that is designed to boost pre-existing tumor-nonspecific or tumor-specific T cells. Such T cells may subsequently infiltrate the tumor microenvironment, which may be pre-modulated by vaccine components. Such infiltration is thought to occur (partially) via a CXCR3-dependent mechanism. By boosting such pre-existing T cells, a larger and more activated pool of T cells may become available for engagement by T-cell engaging bsAbs that can also monovalently bind a tumor target. While such T cells may induce killing of tumor cells upon binding to a T-cell engaging bsAb, the influx of activated T cells and reduction in frequency of suppressive cell populations in the tumor microenvironment is paralleled with activation of tumor-infiltrated NK cells. Together these factors contribute to improved anti-tumor efficacy of the T-cell engaging bsAb treatment.

OT1

1. A method for preventing or reducing growth of a tumor, or for treatment of cancer in a subject in need thereof, comprising providing to the subject
i) a binding agent comprising an antigen-binding region that binds to CD3, such as human CD3, and an antigen-binding region that binds to a target antigen on cells of said tumor or cancer; and
ii) an immunogenic composition comprising at least one vaccine antigen.

2. The method according to claim 1, wherein the vaccine antigen is a non-tumor specific vaccine antigen.

3. The method according to any of the preceding claims, wherein the immunogenic composition is a vaccine, such as a prophylactic vaccine or a therapeutic vaccine.

4. The method according to any one of the preceding claims, wherein said immunogenic composition is a vaccine against an infectious disease; e.g. a viral or bacterial infection.

5. The method according to claim 4, wherein said infectious disease or infection is selected from the group consisting of cholera (V. cholerae; e.g. WC/rBS, Diphtheria (Corynebacterium diphtheriae), Haemophilus influenzae type b (Hib), hepatitis A, hepatitis B, human papillomavirus, influenza, coronavirus, encephalitis, measles, Lymphocytic choriomeningitis (LCM) (lymphocytic choriomeningitis mammarenavirus (LCMV)), meningococcal disease (Neisseria meningitidis), mumps, pertussis/whooping cough (Bordetella pertussis), pneumococcal disease/infections (Streptococcus pneumoniae), poliomyelitis (polio), rabies, rotavirus infection, rubella/German measles, tetanus (Clostridium tetani), smallpox, typhoid fever (Salmonella typhi), varicella/chickenpox, yellow fever, tuberculosis, plaque (Yersinia pestis), bat lyssavirus, Japanese encephalitis, Q fever (Coxiella burnetiid), varicella-zoster (chickenpox), anthrax (Bacillus anthracis).

6. The method according to claim 4, wherein the infection is a coronavirus infection, such as severe acute respiratory syndrome coronavirus 2 (SARS-CoV-2) infection.

7. The method according to claim 4 or 5, wherein the infectious disease is Coronavirus disease 2019 (COVID-19).

8. The method according to claim 4, wherein the infection is influenza infection.

9. The method according to claim 4 or 8, wherein the infectious disease is influenza.

10. The method according to claim 4, wherein the infection is lymphocytic choriomeningitis mammarenavirus (LCMV) infection.

11. The method according to claim 4 or 10, wherein the infectious disease is lymphocytic choriomeningitis (LCM).

12. The method according to any one of the preceding claims, wherein said immunogenic composition is selected from the group consisting of
a live vaccine, such as a live attenuated vaccine,
an inactivated vaccine,
a split vaccine, such as a split virus vaccine,
a vaccine based on virus-like particles,
a fractional vaccine, such as a subunit vaccine, a protein-based vaccine, a peptide-based vaccine, a polysaccharide-based vaccine, or a conjugated vaccine,
a recombinant vaccine; and
a nucleic acid based vaccine, such as an viral vector-based vaccine.

13. The method according to any of claims 1-12, wherein said immunogenic composition is a live vaccine, e.g. a live attenuated vaccine; such as
a live attenuated viral vaccine; e.g. live attenuated measles vaccine, live attenuated mumps vaccine, live attenuated rubella vaccine, live attenuated influenza vaccine, live attenuated chicken pox/varicella-zoster vaccine, live attenuated vaccinia (smallpox) vaccine, live attenuated oral polio vaccine (OPV)(Sabin), live attenuated rotavirus vaccine or live attenuated yellow fever vaccine;
or such as
a live attenuated bacterial vaccine; e.g. BCG vaccine, a live attenuated vaccine against Salmonella typhi (Ty21) (oral typhoid vaccine or epidemic typhus vaccine), a live attenuated cholera vaccine.

14. The method according to any one of claims 1-12, wherein the immunogenic composition is an inactivated vaccine; such as
an inactivated virus vaccine; e.g. inactivated poliovirus vaccine (IPV) (Salk vaccine), inactivated influenza vaccine, inactivated rabies vaccine, inactivated Japanese encephalitis vaccine or an inactivated hepatitis A vaccine;
or such as
an inactivated bacterial vaccine, e.g. inactivated typhoid vaccine, inactivated cholera vaccine, inactivated plague vaccine, inactivated Q fever vaccine, inactivated anthrax vaccine or inactivated pertussis vaccine.

15. The method according to claim 12, wherein the fractional vaccine is a protein-based vaccine selected from the group consisting of
a toxid vaccine, e.g. a vaccine comprising a tetanus toxoid or a diphtheria toxoid;
a subunit vaccine; and
a subvirion vaccine.

16. The method according to claim 12 or 15, wherein the fractional vaccine is a polysaccharide-based vaccine; e.g. polysaccharide-based meningococcal disease (Neisseria meningiditis (A, C, Y.W135)) vaccine, a polysaccharide-based Salmonella typhi vaccine or polysaccharide-based pneumococcal disease (Streptococcus pneumoniae) vaccine.

17. The method according to any one of claims 12, 15 and 16, wherein the fractional vaccine is a conjugated vaccine comprising a polysaccharide linked to a polypeptide; e.g. conjugated Haemophilia influenza type b (Hib) vaccine, conjugated Streptococcus pneumoniae vaccine, conjugated Neisseria meningiditis vaccine.

18. The method according to any one of claims 1-12, wherein the immunogenic composition is a recombinant vaccine.

19. The method according to any of claims 1-12, wherein the immunogenic composition is a vaccine selected from the group consisting of: BCG; Cholera: inactivated oral; Dengue Tetravalent (live, attenuated); Diphtheria-Tetanus; Diphtheria-Tetanus (reduced antigen content); Diphtheria-Tetanus-Pertussis (acellular) (DTaP/Tdap); Diphtheria, Tetanus, acellular Pertussis and Haemophilus influenzae type b (DTaP-Hib); Diphtheria-Tetanus-Pertussis (acellular)-Hepatitis B-Haemophilus influenzae type b-Polio (Inactivated); Diphtheria-Tetanus-Pertussis (whole cell) (DTP); Diphtheria-Tetanus-Pertussis (whole cell)-Haemophilus influenzae type b (DTP-Hib); Diphtheria-Tetanus-Pertussis (whole cell)-Hepatitis B; Diphtheria-Tetanus-Pertussis (whole cell)-Hepatitis B-Haemophilus influenzae type b; Ebola Zaire (rVSVΔG-ZEBOV-GP, live attenuated); Haemophilus influenzae type b (Hib); Hepatitis A (Human Diploid Cell), Inactivated (Adult); Hepatitis A (Human Diploid Cell), Inactivated (Paediatric); Hepatitis B, Hepatitis B (Paediatric); Human Papillomavirus (Bivalent); Human Papillomavirus (Ninevalent); Human Papillomavirus (Quadrivalent); Influenza, pandemic H1N1; Influenza, seasonal (Quadrivalent); Influenza, seasonal (Trivalent); Japanese Encephalitis Vaccine (Inactivated); Japanese Encephalitis Vaccine (live, attenuated); Measles; Measles and Rubella; Measles, Mumps and Rubella (MMR); Measles, Mumps, Rubella and Varicella (MMRV) Measles, Mumps and Rubella (MMR); Meningococcal A Conjugate 10 μg; Meningococcal A Conjugate 5 μg; Meningococcal ACYW-135 (conjugate vaccine); Pneumococcal (conjugate); Polio Vaccine—Inactivated (IPV); Polio Vaccine—Oral (OPV) Bivalent Types 1 and 3; Polio Vaccine—Oral (OPV) Monovalent Type 1; Polio Vaccine—Oral (OPV) Trivalent; Rabies; Rotavirus; Rotavirus (live, attenuated); Rubella; Tetanus Toxoid; Typhoid (Conjugate); Typhoid (Polysaccharide); Varicella; Yellow Fever, Bacille Calmette-Guerin (BCG).

20. The method according to claim 19, wherein the immunogenic composition is a COVID-19 vaccine.

21. The method according to claim 19 or 20, wherein the COVID-19 vaccine is selected from the group consisting of BNT162b2/COMIRNATY (Tozinameran) (Pfizer BioNTech), CVnCoV/CV07050101 (Zorecimeran) (CureVac), AZD1222 Vaxzevria (AstraZeneca), mRNA-1273 (Moderna), SARS-CoV-2 Vaccine (Vero Cell), Inactivated (InCoV) (Sinopharm/Beijing Institute of Biological Products Co., Ltd. (BIBP), CoV2 preS dTM-AS03 vaccine (Sanofi) and Covishield (ChAdOx1_nCoV-19) (Serum Institute of India Pvt. Ltd), Sputnik V (rAd26 and rAd5) (Acellena), Sputnik Light (rAd26) (Acellena), Ad26.COV2.S (JNJ78436735) (Janssen), CoronaVav (Sinovac), BBIBP-CorV (Beijing Institute of Biological Products; China National Pharmaceutical Group (Sinopharm)), EpiVacCorona (Federal Budgetary Research Institution State Research Center of Virology and Biotechnology), Convidicea (CanSino Biologics) and MVC-COV1901 (Medigen Vaccine Biologics Corp.; Dynavax).

22. The method according to claim 19, wherein the immunogenic composition is an influenza vaccine.

23. The method according to claim 19 or 22, wherein the immunogenic composition is an influenza vaccine of a type selected from the group consisting of Influenza Pandemic (H1N1), Influenza seasonal (Trivalent), and Influenza seasonal (Quadrivalent).

24. The method according to claim 19, wherein the infectious disease is lymphocytic choriomeningitis (LCM) vaccine.

25. The method according to any one of the preceding claims, wherein said immunogenic composition is a pediatric vaccine or childhood vaccine.

26. The method according to any one of the preceding claims, wherein said immunogenic composition is a childhood booster vaccine.

27. The method according to claim 1 or 3, wherein said immunogenic composition is a cancer vaccine, such as a prophylactic or therapeutic cancer vaccine.

28. The method according to any one of claims 1, 3 and 27, wherein said vaccine antigen is a tumor-associated antigen or a tumor-specific antigen.

29. The method according to any one of claims 1, 3, 27 and 28, wherein the immunogenic composition comprises a vaccine antigen expressed by said tumor or cancer.

30. The method according to any one of claims 1, 3 and 27-29, wherein the target antigen and the vaccine antigen are both expressed by the said tumor or cancer.

31. The method according to any one of claims 1, 3 and 27-30, wherein the binding angent and the immunogenic composition are directed against or target the same tumor or cancer.

32. The method according to any one of the preceding claims, wherein the vaccine antigen and the target antigen are the same, or the vaccine antigen is a part, a subsequence or a variant of the target antigen.

33. The method according to any one of claims 1, 3 and 27-32, wherein said immunogenic composition comprises a vaccine antigen selected from the group consisting of an antigen that is overexpressed by said tumor, such as Her2/neu, survivin or Muc-1; a cancer neoantigen, such as a p53 neoantigen; a cancer testis antigen, such as MAGE-A3, MAGE-A2, MAGE-A4, PRAME, CT83, SSX2 or NY-ESO-1; a differentiation antigen, such as Mart1, PSA or PAP, and a viral-associated antigen, such as a HPV antigen.

34. The method according to ny one of claims 1, 3 and 27-32, wherein the immunogenic composition is a personalized cancer vaccne and the vaccine antigen is a neoantigen, which is specific to the subject's tumor.

35. The method according to any of the preceding claims, wherein said vaccine antigen comprises a T-cell epitope.

36. The method according to any one of the preceding claims, wherein said immunogenic composition is capable of eliciting a cytotoxic T-cell response in vivo and/or in vitro.

37. The method according to any on of the preceding claims, wherein said vaccine is capable of eliciting a T-cell response, which comprises T-cell activation and/or T-cell infiltration of said tumor or cancer.

38. The method according to any on of the preceding claims, wherein said immunogenic composition is capable of i) eliciting a T-cell response, which comprises T-cell infiltration of said tumor or cancer and activation of tumor-infiltrating T-cells; and/or ii) eliciting infiltration and/or expansion of immune cells in tumors, such as infiltration and/or expansion natural killer (NK) cells and/or dendritic cells (DCs).

39. The method according to claim 37 or 38, wherein T-cell infiltration and/or activation is determined in mice carrying a tumor that expresses an antigen to which the 2^{nd} binding region of said binding agent binds, using a procedure in which the mice are subject to transfer of tumor specific T-cells and then injected subcutaneously with the immunogenic composition and subsequently with the binding agent.

40. The method according to any one of claims 37-39, wherein infiltration and/or activation is determined in a procedure comprising the steps of:
i) Providing T cells which express an antigen to which the binding agent is capable of binding, and constitutively express a 1^{st} oxidative enzyme capable of producing a 1^{st} bioluminescense and 2^{nd} oxidative enzyme capable of producing a 2^{nd} bioluminescense, wherein expression of the 2^{nd} oxidative is induced by T-cell activation, such as by Nuclear Factor of Activated T cells (NFAT),
ii) Injecting a mouse, such as an Albino C57BL/6 mice, carrying a tumor that expresses an antigen to which the tumor-targeting binding region of said binding agent binds, with the T cell defined in i) by intravenous injection in the tail vein,
iii) Administering to the mouse two doses of the immunogenic composition by subcutaneous injection at the tail base, 1 and 8 days after injection of the T cells in ii),
iv) Administering to the mouse two doses of said binding agent by intravenous injection or infusion, 10 and 14 days after injection of the T cells in ii),
v) Injecting the mouse with a substrate of each of said oxidative enzymes and measuring said 1^{st} and 2^{nd} bioluminiscense.

41. The method according to any one of claims 37-39, wherein T-cell infiltration and/or activation is determined using a procedure, which is essentially as set forth in Example 5 herein, or using the procedure set forth in Example 5.

42. The method according to any one of the preceding claims, wherein the method comprises determining existing T-cell immunity in said subject; such as existing T-cell immunity from previous vaccination or infection.

43. The method according to claim 42, wherein existing T-cell immunity is determined by measurement of peripheral T-cell activation.

44. The method according to any one of the preceding claims, wherein the method comprises determining the subject's previous participation in a vaccination program, such as a childhood vaccination program.

45. The method according to any one of the preceding claims, wherein the immunogenic composition is a vaccine against an infection or infectious disesase, which the subject has previously had, and/or has developed a T-cell immune response against; such as an infection or infectious disease or infection defined in any one of claims.

46. The method according to any one of the preceding claims, wherein the method further comprises administration of an adjuvant.

47. The method according to claim 46, wherein the adjuvant is a Th1/Th2 adjuvant, a Th1 adjuvant or a Th2 adjuvant; preferably a Th1 adjuvant.

48. The method according to any one of the preceding claims, wherein the immunogenic composition when administered with the adjuvant is capable of eliciting a Th1/Th2-type immune response, a Th1-type immune response or a Th2-type immune response; preferably a Th1-type immune response.

49. The method according to any one of the preceding claims, wherein the immunogenic composition comprises an adjuvant.

50. The method according to claim 49, wherein the immunogenic composition comprising the adjuvant is capable of eliciting an NK cell response and/or a Th1/Th2-type immune response, a Th1-type immune response, or a Th2-type immune response; preferably a Th1-type immune response.

51. The method according to any one of claims 46-50, wherein the adjuvant comprises an aluminum salt; e.g. alum (XAl(SO₄)₂·12H₂O; where X is a monovalent cation, such as K⁺ or NH₄⁺).

52. The method according to any one of claims 46-50, wherein the adjuvant is an emulsion-based adjuvant, such as an oil-in-water emulsion.

53. The method according to any one of claims 46-50 and 52, wherein the adjuvant is selected from the group consisting of
a) an adjuvant comprising squalene, polysorbate 80 and sorbitan trioleate; e.g. MF59,
b) an adjuvant comprising squalene, polysorbate 80 and α-tocopherol; e.g. AS03
c) an adjuvant comprising squalene, polyoxyethylene, cetostearyl ether, mannitol and sorbitan oleate; e.g. AF03.
d) an adjuvant comprising 3-O-desacyl-4′-monophosphoryl lipid A (MPL), Quillaja saponaria Molina, fraction 21 (QS-21) and liposome; e.g. AS01; and
e) an adjuvant comprising 3-O-desacyl-4′-monophosphoryl lipid A (MPL) and aluminium hydroxide; e.g. AS04.

54. The method according to claim 46 or 49, wherein the adjuvant comprises a Toll-like receptor 7 (TLR7) agonist, such as a TLR7 agonist selected from the group consisting of Imiquimod (Aldara), resiquimod and gardiquimod.

55. The method according to claim 46 or 49, wherein the adjuvant comprises
i) a Toll-like receptor 9 (TLR9) agonist, such as a TLR9 agonist selected from the group consisting of Neisseria meningitidis porin B (porB), a CpG Oligodeoxynucleotide (ODN) and tilsotolimod,
ii) a Toll-like receptor 4 (TLR4) agonist, such as a TLR4 agonist selected from the group consisting of monophosphoryl lipid A (MPL), glucopyranosyl lipid A (GLA) and neoseptin-3,
iii) a Toll-like receptor 5 (TLR5) agonist, such as a TLR5 agonist selected from the group consisting of mobilan, entolimod or recombinant flagellin FlicC; and/or
iv) a Toll-like receptor 3 (TLR3) agonist, such as a TLR3 agonist selected from the group consisting of Poly-IC and derivatives thereof.

56. The method according to any one of claims 1-49, wherein the adjuvant comprises nanoparticles, such as lipid nanoparticles (LPNs); e.g. adjuvant-incorporated lipid nanoparticles.

57. The method according to any one of the preceding claims, wherein the immunogenic composition or the adjuvant comprises a cytokine.

58. The method according to any one of the preceding claims, wherein the method comprises administering the immunogenic composition in combination with a cytokine.

59. The method according to claim 57 or 58, wherein the cytokine is an interleukin-2 (IL2) receptor agonist, such as IL2/aldesleukin or interleukin-15.

60. The method according to any one of the preceding claims, wherein the binding agent is administered to the subject by parenteral or systemic administration, such as by injection or infusion; e.g. by intravenous injection or infusion.

61. The method according to any one of the preceding claims, wherein the binding agent is provided to the subject in one or more treatment cycles.

62. The method according to any one of the preceding claims, wherein the binding agent is dosed once a week (1Q1W), once every second week (1Q2W), once every third week (1Q3W) or once every fourth week (1Q4W).

63. The method according to any one of the preceding claims, wherein the immunogenic composition is administered to the subject to achieve a topical effect/by topical administration, such as by application of a cream onto the skin.

64. The method according to any one of claims 1-62, wherein the immunogenic composition is administered to achieve a systemic effect/by systemic administration, such as by oral administration, subcutaneous injection, intramuscular injection, intradermal injection or by a transmucosal route.

65. The method according to any of the preceding claims, wherein the adjuvant is is administered to the subject to achieve a topical effect/by topical administration, such as by application of a cream onto the skin.

66. The method according to any one of claims 46-64, wherein the adjuvant is administered to achieve a systemic effect/by systemic administration, such as by oral administration, subcutaneous injection, intramuscular injection, intradermal injection or by a transmucosal route.

67. The method according to any one of claims 58-66, wherein the cytokine, such as the interleukin 2 receptor agonist, is administered to the subject by parenteral or systemic administration, such as by injection or infusion; e.g. by intravenous injection or infusion.

68. The method according to any one of the preceding claims, wherein the immunogenic composition and optionally the adjuvant and/or the cytokine, such as the interleukin 2 receptor agonist, is/are administered as part of a treatment regimen provided to reduce growth of said tumor, and/or to treat said cancer.

69. The method according to any one of the preceding claims, wherein administration of the immunogenic composition and the binding agent is combined with adoptive T-cell therapy.

70. The method according to any one of the preceding claims, comprising administering a plurality of T-cells to the subject.

71. The method according to any one of the preceding claims, wherein the immunogenic composition is administered to said subject simultaneously with or on the same day as administration of the binding agent, simultaneously with or on the same day as administration of the first dose of the binding agent and/or as part of the first treatment cycle with the binding agent.

72. The method according to any one of claims 1-71, wherein administration of the immunogenic composition and administration of the binding agent, such as administration of the immunogenic composition and administration of the first dose of the binding agent and/or onset of the first treatment cycle with the binding agent, is separated by a time period of at the most 2 months, such as at the most 1 month, at the most 4 weeks, at the most 3 weeks, at the most 2 weeks, at the most 1 week, at the most 6 days, at the most 5 days, at the most 4 days, at the most 3, days or at the most 2 days.

73. The method according to any one of claims 1-71, wherein administration of the immunogenic composition and administration of the binding agent, such as administration of the immunogenic composition and administration of the first dose of the binding agent and/or onset of the first treatment cycle with the binding agent, is separated by a time period of 2 days to 2 months, such as 2 days to 1 month, 2 days to 4 weeks, 2 days to 3 weeks, 2 days to 2 weeks, 2 days to 1 week, 3 days to 2 months, 3 days to 1 month, 3 days to 4 weeks, 3 days to 3 weeks, 3 days to 2 weeks, 3 days to 1 week, 4 days to 2 months, 4 days to 1 month, 4 days to 4 weeks, 4 days to 3 weeks, 4 days to 2 weeks, 4 days to 1 week, 5 days to 2 months, 5 days to 1 month, 5 days to 4 weeks, 5 days to 3 weeks, 5 days to 2 weeks, 5 days to 1 week, 6 days to 2 months, such as 6 days to 1 month, 6 days to 4 weeks, 6 days to 3 weeks, 6 days to 2 weeks, 1 week to 2 months, 1 week to 1 month, 1 to 4 weeks, 1 to 3 weeks, 1 to 2 weeks, 3 weeks to 2 months, 3 weeks to 1 month or 3 to 4 weeks.

74. The method according to any one of claims 1-71, wherein the immunogenic composition is administered to said subject prior to administration of the binding agent, prior to administration of a first dosage of the binding agent and/or prior to the first treatment cycle with the binding agent.

75. The method according to any one of claims 1-71 and 74, wherein the immunogenic composition is administered to the subject from 2 days to 2 months prior to aministration of the binding agent, prior to administration of a first dosage of the binding agent and/or prior to the first treatment cycle with the binding agent; such as from 2 days to 1 month, from 2 days to 4 weeks, from 2 days to 3 weeks, from 2 days to 2 weeks, from 2 days to 1 week, from 3 days to 2 months, from 3 days to 1 month, from 3 days to 4 weeks, from 3 days to 3 weeks, from 3 days to 2 weeks, from 3 days to 1 week, from 4 days to 2 months, from 4 days to 1 month, from 4 days to 4 weeks, from 4 days to 3 weeks, from 4 days to 2 weeks, from 4 days to 1 week, from 5 days to 2 months, from 5 days to 1 month, from 5 days to 4 weeks, from 5 days to 3 weeks, from 5 days to 2 weeks, from 5 days to 1 week, from 6 days to 2 months, from 6 days to 1 month, from 6 days to 4 weeks, from 6 days to 3 weeks, from 6 days to 2 weeks, from 1 week to 2 months, from 1 week to 1 month, from 1 to 4 weeks, from 1 to 3 weeks, from 1 to 2 weeks, from 3 weeks to 2 months, from 3 weeks to 1 month, or such as from 3 to 4 weeks prior to aministration of the binding agent, prior to administration of a first dosage of the binding agent and/or prior to the first treatment cycle with the binding agent.

76. The method according to any one of claims 65-75, wherein the plurality of T-cells is administered to said subject, or the adoptive T-cell theray is provided to said subject, simultaneously with or on the same day as administration of the binding agent, simultaneously with or on the same day as administration of the first dose of the binding agent and/or as part of the first treatment cycle with the binding agent.

77. The method according to any one of claims 65-75, wherein the plurality of T-cells is administered to said subject, or the adoptive T-cell theray is provided to said subject, prior to administration of the binding agent, prior to administration of a first dosage of the binding agent and/or prior to the first treatment cycle with the binding agent.

78. The method according to any one of claims 65-75, wherein
i) administration of the immunogenic composition and administration of the plurality of T-cells, or the adoptive T-cell therapy, and
ii) administration of the binding agent, such as administration of the first dose of the binding agent and/or onset of the first treatment cycle with the binding agent,
are separated by a time period of 2 days to 2 months, such as 2 days to 1 month, 2 days to 4 weeks, 2 days to 3 weeks, 2 days to 2 weeks, 2 days to 1 week, 3 days to 2 months, 3 days to 1 month, 3 days to 4 weeks, 3 days to 3 weeks, 3 days to 2 weeks, 3 days to 1 week, 4 days to 2 months, 4 days to 1 month, 4 days to 4 weeks, 4 days to 3 weeks, 4 days to 2 weeks, 4 days to 1 week, 5 days to 2 months, 5 days to 1 month, 5 days to 4 weeks, 5 days to 3 weeks, 5 days to 2 weeks, 5 days to 1 week, 6 days to 2 months, such as 6 days to 1 month, 6 days to 4 weeks, 6 days to 3 weeks, 6 days to 2 weeks, 1 week to 2 months, 1 week to 1 month, 1 to 4 weeks, 1 to 3 weeks, 1 to 2 weeks, 3 weeks to 2 months, 3 weeks to 1 month or 3 to 4 weeks.

79. The method according to any one of claims 65-75, wherein the immunogenic composition and administration of the plurality of T-cells, or the adoptive T-cell therapy are administered to the subject from 2 days to 2 months prior to aministration of the binding agent, prior to administration of a first dosage of the binding agent and/or prior to the first treatment cycle with the binding agent; such as from 2 days to 1 month, from 2 days to 4 weeks, from 2 days to 3 weeks, from 2 days to 2 weeks, from 2 days to 1 week, from 3 days to 2 months, from 3 days to 1 month, from 3 days to 4 weeks, from 3 days to 3 weeks, from 3 days to 2 weeks, from 3 days to 1 week, from 4 days to 2 months, from 4 days to 1 month, from 4 days to 4 weeks, from 4 days to 3 weeks, from 4 days to 2 weeks, from 4 days to 1 week, from 5 days to 2 months, from 5 days to 1 month, from 5 days to 4 weeks, from 5 days to 3 weeks, from 5 days to 2 weeks, from 5 days to 1 week, from 6 days to 2 months, from 6 days to 1 month, from 6 days to 4 weeks, from 6 days to 3 weeks, from 6 days to 2 weeks, from 1 week to 2 months, from 1 week to 1 month, from 1 to 4 weeks, from 1 to 3 weeks, from 1 to 2 weeks, from 3 weeks to 2 months, from 3 weeks to 1 month, or such as from 3 to 4 weeks prior to aministration of the binding agent, prior to administration of a first dosage of the binding agent and/or prior to the first treatment cycle with the binding agent.

80. The method according to any one of claims 65-79, wherein each of the binding agent, the immunogenic composition and, optionally, the adjuvant, the plurality of T-cells and interlukin 2, is provided to the subject in an effective amount.

81. The method according to any of claims 1-66, wherein a first dosage of the binding agent is administered to said subject prior to administration of the immunogenic composition and/or wherein the immunogenic composition is administered as part of the second or subsequent treatment schedules with said binding agent.

82. The method according to any of the preceding claims, wherein the method comprises determining whether there is a T-cell specific response to the immunogenic composition in the subject and/or monitoring any T-cell specific response to the immunogenic composition, optionally including monitoring expansion in said subject of T cells specific to the immunogenic composition.

83. The method according to any one of the preceding claims, wherein the target antigen is an antigen, which is specific for said tumor.

84. The method according to any one of the preceding claims, wherein said target antigen is an antigen, which is overexpressed by cells of said tumor or cancer; such as overexpressed when compared with cells of healthy tissue.

85. The method according to any one of claims 1-83, wherein said target antigen is an antigen which is expressed exclusively by cells of said tumor or cancer, or is an antigen overexpressed on immunosuppressive cells within the tumor microenvironment (e.g. TAMs, MDSCs, Treg).

86. The method according to any one of the preceding claims, wherein the target antigen is selected from the group consisting of Her2, CD19, EpCAM, EGFR, CD66e (CEA, CEACAM5), CD33, EphA2 and MCSP (HMW-MAA).

87. The method according to claim 86, wherein the antigen-binding region that binds to said target antigen is selected from the group consisting of: an antigen-binding region of Herceptin that binds to Her2/neu, an antigen-binding region of Blinatumomab that binds to CD19, an antigen-binding region of catumaxomab that binds to EpCAM, an antigen-binding region of cetuximab or panitumumab that binds to EGFR, and an antigen-binding region of Lintuzumab that binds to CD33.

88. The method according to any one of the preceding claims, wherein the tumor is a solid tumor.

89. The method according to any one of the preceding claims, wherein the tumor or cancer is selected from the group consisting of breast cancer, prostate cancer, non-small cell lung cancer, bladder cancer, ovarian cancer, gastric cancer, colorectal cancer, esophageal cancer and squamous cell carcinoma of the head & neck, cervical cancer, pancreatic cancer, testis cancer, malignant melanoma, a soft-tissue cancer; e.g. synovial sarcoma.

90. The method according to any one of the preceding claims, wherein the tumor is selected from the group consisting of melanoma, and adenocarcinoma (e.g. ductal adenocarcinoma).

91. The method according to any one of claims 1-87, wherein the tumor is a hematologic tumor.

92. The method according to claim 91, wherein the hematologic tumor is selected from the group consisting of B-cell lymphoma, and chronic lymphatic leukemia or acute lymphatic leukemia.

93. The method according to any one of the preceding claims, wherein the tumor is devoid of any immune infiltrate, such as devoid of any functional immune filtrate.

94. The method according to any one of the preceding claims, wherein the tumor is characterized by the presence of suppressive immune cells such as immune cells selected from the group consisting of regulatory T cells (Tregs), myeloid-derived suppressor cells (MDSCs) and M2-type macrophages.

95. The method according to any one of the preceding claims, wherein the antigen-binding region that binds to CD3, binds to human CD3ε (epsilon), such as human CD3ε (epsilon) as specified in SEQ ID NO: 1.

96. The method according to any one of the preceding claims, wherein the antigen-binding region that binds to CD3 comprises
a heavy chain variable region (VH) comprising the CDR1, CDR2, and CDR3 sequences of SEQ ID NOs.: 2, 3 and 4, respectively; [wildtype anti-CD3 (SP34/humanized SP34, WO2015001085 (Genmab))—VH CDR sequences];
and, optionally
a light chain variable region (VL) comprising comprising the CDR1, CDR2, and CDR3 sequences of SEQ ID NO: 6, GTN and 7, respectively [wildtype anti-CD3, VL CDR sequences].

97. The method according to any one of the preceding claims, wherein the antigen-binding region that binds to CD3 comprises
a heavy chain variable region (VH) comprising the sequence of SEQ ID NO: 57, or a sequence having at least 90%, at least 95%, at least 97%, or at least 99% amino acid sequence identity to the sequence of SEQ ID NO: 5 [wildtype anti-CD3—VH full length sequence];
and, optionally
a light chain variable region (VL) comprising the sequence of SEQ ID NO: 60 or a sequence having at least 90%, at least 95%, at least 97%, or at least 99% amino acid sequence identity to the sequence of SEQ ID NO: 8, [wild type anti-CD3—VL full length sequence].

98. The method according to any one of claims 1-95, wherein said binding agent has a lower human CD3ε binding affinity than a binding agent having an antigen-binding region comprising a VH sequence as set forth in SEQ ID NO: 5, and a VL sequence as set forth in SEQ ID NO: 8 [wild type anti-CD3 (humanized SP34, WO2015001085 (Genmab)) VH and VL sequences], preferably wherein said affinity is at least 5-fold lower, such as at least 10-fold lower, e.g. at least 20-fold lower, at least 30 fold lower, at least 40 fold lower, at least 45 fold lower or such as at least 50-fold lower.

99. The method according to any one of the preceding claims 1-96, wherein said antigen-binding region that binds to CD3 with an equilibrium dissociation constant K_{D} within the range of 200-1000 nM, such as within the range of 300-1000 nM, within the range of 400-1000 nM, within the range of 500-1000 nM, within the range of 300-900 nM within the range of 400-900 nM, within the range of 400-700 nM, within the range of 500-900 nM, within the range of 500-800 nM, within the range of 500-700 nM, within the range of 600-1000 nM, within the range of 600-900 nM, within the range of 600-800 nM, or such as within the range of 600-700 nM.

100. The method according to any one of claims 1-97, wherein said antigen binding-region that binds to CD3 with an equilibrium dissociation constant K_{D} within the range of 1-100 nM, such as within the range of 5-100 nM, within the range of 10-100 nM, within the range of 1-80 nM, within the range of 1-60 nM within the range of 1-40 nM, within the range of 1-20 nM, within the range of 5-80 nM, within the range of 5-60 nM, within the range of 5-40 nM, within the range of 5-20 nM, within the range of 10-80 nM, within the range of 10-60 nM, within the range of 10-40 nM, or such as within the range of 10-20 nM.

101. The method according to any one of claims 1-95, wherein
the antigen binding region that binds to CD3 comprises a heavy chain variable (VH) region comprising a CDR1 sequence, a CDR2 sequence and a CDR3 sequence,
the heavy chain variable (VH) region, when compared to a heavy chain variable (VH) region comprising the sequence set forth in SEQ ID NO: 5, has an amino acid substitution in one of the CDR sequences, the substitution being at a position selected from the group consisting of: T31, N57, H101, G105, S110 and Y114, the positions being numbered according to the sequence of SEQ ID NO: 5 [VH_huCD3-H1L1]; and
the wild type light chain variable (VL) region comprises the CDR1, CDR2 and CDR3 sequences set forth in SEQ ID NO: 6, GTN and SEQ ID NO: 7, respectively.

102. The method according to claim 101, wherein the CDR1, CDR2 and CDR3 of the heavy chain variable (VH) region of the antigen binding region that binds to CD3 comprises, in total, at the most 1, 2, 3, 4 or 5 amino acid substitutions, when compared to the CDR1, CDR2 and CDR3 of the sequence set forth in SEQ ID NO: 5.

103. The method according to claim 101 or 102, wherein the amino acid sequences of the CDR1, CDR2 and CDR3 of the heavy chain variable (VH) region of the antigen-binding region that binds to CD3 have at least 95% sequence identity, such as at least 96% sequence identity, at least 97% sequence identity, at least 98% sequence identity or at least 99% sequence identity to the amino acid sequences of the CDR1, CDR2 and CDR3 of the wild type heavy chain variable (VH) region, sequence identity being calculated based on an aligning an amino acid sequence consisting of the sequences of the CDR1, CDR2 and CDR3 of the heavy chain variable (VH) region of the antigen binding region that binds to CD with an amino acid sequence comprising the sequences of the CDR1, CDR2 and CDR3 of the wild type heavy chain variable (VH) region.

104. The method according to any one of claims 1-95 and 98-103, wherein the antigen-binding region that binds to CD3 comprises a mutation selected from the group consisting of: T31M, T31P, N57E, H101G, H101N, G105P, S110A, S110G, Y114M, Y114R, Y114V.

105. The method according to any one of claims 1-95 and 98-104, wherein the antigen-binding region capable of binding to CD3 comprises:
a) a heavy chain variable region (VH) comprising CDR1, CDR2, and CDR3 having the sequences as set forth in SEQ ID NOs: 9, 3, and 4 [VH CDR1-T31P+Wild type VH CDRs 2,3], respectively, and a light chain variable region (VL) comprising CDR1, CDR2, and CDR3 having the sequences as set forth in SEQ ID NO: 6, the sequence GTN, and the sequence as set forth in SEQ ID NO: 7, respectively [Wild type VL CDRs 1,2,3], or
b) a heavy chain variable region (VH) comprising CDR1, CDR2, and CDR3 having the sequences as set forth in SEQ ID NOs: 11, 3, and 4 [VH CDR1-T31M+Wild type VH CDRs 2,3], respectively, and a light chain variable region (VL) comprising CDR1, CDR2, and CDR3 having the sequences as set forth in SEQ ID NO: 6, the sequence GTN, and the sequence as set forth in SEQ ID NO: 7, respectively [Wild type VL CDRs 1,2,3], respectively, or
c) a heavy chain variable region (VH) comprising CDR1, CDR2, and CDR3 having the sequences as set forth in SEQ ID NOs: 2, 13, and 4 [VH CDR-N57E+Wild type VH CDRs 1,3], respectively, and a light chain variable region (VL) comprising CDR1, CDR2, and CDR3 having the sequences as set forth in SEQ ID NO: 6, the sequence GTN, and the sequence as set forth in SEQ ID NO: 7, respectively [Wild type VL CDRs 1,2,3], respectively, or
d) a heavy chain variable region (VH) comprising CDR1, CDR2, and CDR3 having the sequences as set forth in SEQ ID NOs: 2, 3, and 15 [Wild type VH CDRs 1,2+VH CDR3-H101G], respectively, and a light chain variable region (VL) comprising CDR1, CDR2, and CDR3 having the sequences as set forth in SEQ ID NO: 6, the sequence GTN, and the sequence as set forth in SEQ ID NO: 7, respectively [Wild type VL CDRs 1,2,3], respectively.
e) a heavy chain variable region (VH) comprising CDR1, CDR2, and CDR3 having the sequences as set forth in SEQ ID NOs: 2, 3, and 17 [Wild type VH CDRs 1,2+VH CDR3-H101N], respectively, and a light chain variable region (VL) comprising CDR1, CDR2, and CDR3 having the sequences as set forth in SEQ ID NO: 6, the sequence GTN, and the sequence as set forth in SEQ ID NO: 7, respectively [Wild type VL CDRs 1,2,3], respectively;
f) a heavy chain variable region (VH) comprising CDR1, CDR2, and CDR3 having the sequences as set forth in SEQ ID NOs: 2, 3, and 19 [Wild type VH CDRs 1,2+VH CDR3-G105P], respectively, and a light chain variable region (VL) comprising CDR1, CDR2, and CDR3 having the sequences as set forth in SEQ ID NO: 6, the sequence GTN, and the sequence as set forth in SEQ ID NO: 7, respectively [Wild type VL CDRs 1,2,3], respectively;
g) a heavy chain variable region (VH) comprising CDR1, CDR2, and CDR3 having the sequences as set forth in SEQ ID NOs: 2, 3, and 21 [Wild type VH CDRs 1,2+VH CDR3-S110A], respectively, and a light chain variable region (VL) comprising CDR1, CDR2, and CDR3 having the sequences as set forth in SEQ ID NO: 6, the sequence GTN, and the sequence as set forth in SEQ ID NO: 7, respectively [Wild type VL CDRs 1,2,3], respectively, or
h) a heavy chain variable region (VH) comprising CDR1, CDR2, and CDR3 having the sequences as set forth in SEQ ID NOs: 2, 3, and 23 [Wild type VH CDRs 1,2+VH CDR3-S110G], respectively, and a light chain variable region (VL) comprising CDR1, CDR2, and CDR3 having the sequences as set forth in SEQ ID NO: 658, the sequence GTN, and the sequence as set forth in SEQ ID NO: 7, respectively [Wild type VL CDRs 1,2,3], respectively,
i) a heavy chain variable region (VH) comprising CDR1, CDR2, and CDR3 having the sequences as set forth in SEQ ID NOs: 2, 3, and 25 [Wild type VH CDRs 1,2+VH CDR3-Y114V], respectively, and a light chain variable region (VL) comprising CDR1, CDR2, and CDR3 having the sequences as set forth in SEQ ID NO: 6, the sequence GTN, and the sequence as set forth in SEQ ID NO: 7, respectively [Wild type VL CDRs 1,2,3], respectively, or
j) a heavy chain variable region (VH) comprising CDR1, CDR2, and CDR3 having the sequences as set forth in SEQ ID NOs: 2, 3, and 27 [Wild type VH CDRs 1,2+VH CDR3-Y114M], respectively, and a light chain variable region (VL) comprising CDR1, CDR2, and CDR3 having the sequences as set forth in SEQ ID NO: 6, the sequence GTN, and the sequence as set forth in SEQ ID NO: 7, respectively [Wild type VL CDRs 1,2,3], respectively, or
k) a heavy chain variable region (VH) comprising CDR1, CDR2, and CDR3 having the sequences as set forth in SEQ ID NOs: 2, 3, and 29 [Wild type VH CDRs 1,2+VH CDR3-Y114R], respectively, and a light chain variable region (VL) comprising CDR1, CDR2, and CDR3 having the sequences as set forth in SEQ ID NO: 6, the sequence GTN, and the sequence as set forth in SEQ ID NO: 7, respectively [Wild type VL CDRs 1,2,3], respectively.

106. The method according to any one of claims 1-83 and 86-93, wherein the antigen-binding region capable of binding to CD3 comprises a heavy chain variable region (VH) comprising CDR1, CDR2, and CDR3 having the sequences as set forth in SEQ ID NOs: 2, 3, and 15 [Wild type VH CDRs 1,2+VH CDR3-H101G], respectively, and a light chain variable region (VL) comprising CDR1, CDR2, and CDR3 having the sequences as set forth in SEQ ID NO: 6, the sequence GTN, and the sequence as set forth in SEQ ID NO: 7, respectively [Wild type VL CDRs 1,2,3], respectively.

107. The method according to any one of the preceding claims, wherein the antigen-binding region capable of binding to human CD3 comprises a VH sequence and a VL sequence selected from the group consisting of:
a) a VH sequence as set forth in SEQ ID NO: 10 [VH T31P full length sequence] and a VL sequence as set forth in SEQ ID NO: 8 [Wild type full length sequence],
b) a VH sequence as set forth in SEQ ID NO: 12 [VH T31M full length sequence] and a VL sequence as set forth in SEQ ID NO: 8,
c) a VH sequence as set forth in SEQ ID NO: 14 [VH N57E full length sequence] and a VL sequence as set forth in SEQ ID NO: 8,
d) a VH sequence as set forth in SEQ ID NO: 16 [VH H101G full length sequence] and a VL sequence as set forth in SEQ ID NO: 8,
e) a VH sequence as set forth in SEQ ID NO: 18 [VH H101N full length sequence] and a VL sequence as set forth in SEQ ID NO: 8,
f) a VH sequence as set forth in SEQ ID NO: 20 [VH G105P full length sequence] and a VL sequence as set forth in SEQ ID NO: 8,
g) a VH sequence as set forth in SEQ ID NO: 22 [VH S110A full length sequence] and a VL sequence as set forth in SEQ ID NO: 8,
h) a VH sequence as set forth in SEQ ID NO: 24 [VH S110G full length sequence] and a VL sequence as set forth in SEQ ID NO: 8,
i) a VH sequence as set forth in SEQ ID NO: 26 [VH Y114V full length sequence] and a VL sequence as set forth in SEQ ID NO: 8,
j) a VH sequence as set forth in SEQ ID NO: 28 [VH Y114M full length sequence] and a VL sequence as set forth in SEQ ID NO: 8; and
k) a VH sequence as set forth in SEQ ID NO: 30 [VH Y114R full length sequence] and a VL sequence as set forth in SEQ ID NO: 8.

108. The method according to any one of claims 1-95 and 98-107, wherein the antigen-binding region capable of binding to human CD3 comprises a VH sequence as set forth in SEQ ID NO: 10 [VH H101G full length sequence] and a VL sequence as set forth in SEQ ID NO: 8.

109. The method according to any one of the preceding claims, wherein the antibody is of an isotype selected from the group consisting of IgG1, IgG2, IgG3 and IgG4.

110. The method according to any one of the preceding claims, wherein the binding agent is a full-length antibody, such as a full length IgG1 antibody.

111. The method according to any one of the preceding claims, wherein the binding agent is an antibody of the IgG1m(f) allotype.

112. The method according to any one of the preceding claims, wherein the binding agent is a multi-specific antibody, such as a bispecific antibody.

113. The method according to any one of the preceding claims, wherein each antigen-binding region comprises a heavy chain variable region (VH) and a light chain variable region (VL), and wherein said variable regions each comprise three CDR sequences, CDR1, CDR2 and CDR3, respectively, and four framework sequences, FR1, FR2, FR3 and FR4, respectively.

114. The method according to claim 113, wherein the binding agent comprises two heavy chain constant regions (CH), and two light chain constant regions (CL).

115. The method according to claim 113 or 114, wherein said binding agent comprises a first and a second heavy chain, each of said first and second heavy chain comprises at least a hinge region, a CH2 and CH3 region, wherein in said first heavy chain at least one of the amino acids in the positions corresponding to positions selected from the group consisting of T366, L368, K370, D399, F405, Y407 and K409 in a human IgG1 heavy chain has been substituted, and in said second heavy chain at least one of the amino acids in the positions corresponding to a position selected from the group consisting of T366, L368, K370, D399, F405, Y407, and K409 in a human IgG1 heavy chain has been substituted, wherein said substitutions of said first and said second heavy chains are not in the same positions, and wherein the amino acid positions are numbered according to EU numbering.

116. The method according to any one of the preceding claims, wherein the binding agent comprises a first and a second heavy chain, and wherein the amino acid in the position corresponding to K409 in a human IgG1 heavy chain is R in said first heavy chain, and the amino acid in the position corresponding to F405 in a human IgG1 heavy chain is L in said second heavy chain, or vice versa.

117. The method according to any one of the preceding claims, wherein the binding agent comprises a first and a second heavy chain, and wherein in both the first and the second heavy chain, the amino acid residues at the positions corresponding to positions L234 and L235 in a human IgG1 heavy chain according to EU numbering are F and E, respectively.

118. The method according to any one of the preceding claims, wherein the binding agent comprises a first and a second heavy chain, and wherein in both the first and the second heavy chain, the amino acid residue at the position corresponding to position D265 in a human IgG1 heavy chain according to EU numbering is A.

119. The method according to any one of the preceding claims, wherein said binding agent comprises a first and, optionally, a second heavy chain and wherein the first heavy chain, and the second heavy chain if present, is/are modified so that the antibody induces Fc-mediated effector function to a lesser extent relative to an identical non-modified antibody.

120. The method according to any one of the preceding claims, wherein said binding agent comprises a kappa (κ) light chain.

121. The method according to any one of the preceding claims, wherein said binding agent comprises a lambda (λ) light chain.

122. The method according to any one of the preceding claims, wherein the binding agent comprises a heavy chain and a lambda (λ) light chain which comprise the binding region that binds to CD3.

123. The method according to any one of claims 1-121, wherein the binding agent comprises a heavy chain and a kappa (κ) light chain which comprise the binding region that binds to CD3.

124. The method according to claim 123, wherein the kappa (κ) light chain comprises an amino acid sequence selected from the group consisting of
a) the sequence set forth in SEQ ID NO: 31,
b) a subsequence of the sequence in a), such as a subsequence wherein 1, 2, 3, 4, 5, 6, 7, 8, 9 or 10 consequtive amino acids has/have deleted, starting from the N-terminus or C-terminus of the sequence defined in a); and
c) a sequence having at the most 5 substitutions, such as at the most 4 substitutions, at the most 3, at the most 2 or at the most 1 substitution compared to the amino acid sequence defined in a) or b).

125. The method according to claim 122, wherein the lambda (λ) light chain comprises an amino acid sequence selected from the group consisting of
a) the sequence set forth in SEQ ID NO: 32,
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 5 substitutions, such as at the most 4 substitutions, at the most 3, at the most 2 or at the most 1 substitution compared to the amino acid sequence defined in a) or b).

126. The method according to any one of the preceding claims, wherein the antibody comprises a fist and/or second heavy chain, and the constant region of said first and/or second heavy chain comprises or consists essentially of or consists of an amino acid sequence selected from the group consisting of
a) the sequence set forth in SEQ ID NO: 33,
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 5 substitutions, such as at the most 4 substitutions, at the most 3, at the most 2 or at the most 1 substitution compared to the amino acid sequence defined in a) or b).

127. The method according to any one of claims 124-126, wherein the at the most 5 substitutions comprise one or more substitutions, such as 1, 2, 3 or 4 substitutions, selected from the group consisting of L234F, L235E, D265A, F405L and K409R.

128. A method for preventing or reducing growth of a tumor in a subject in need thereof, comprising
providing to the subject an immunogenic composition comprising at least one vaccine antigen, thereby increasing the relative amount of immune cells within the live cell population in the tumor, and optionally increasing the activation of said immune cells;
and
providing to the subject a binding agent comprising an antigen-binding region that binds to CD3, such as human CD3, and an antigen-binding region that binds to a target antigen on cells of said tumor or cancer.

129. The method according to claim 128, wherein said immune cells are selected from the group consisting of T cells, such as CD8⁺ T cells, natural killer (NK) cells and natural killer T (NKT) cells.

130. A method for preventing or reducing growth of a tumor, or for treatment of cancer in a subject in need thereof, comprising providing to the subject
i) a nucleic acid construct encoding a binding agent comprising an antigen-binding region that binds to CD3, such as human CD3, and an antigen-binding region that binds to a target antigen on cells of said tumor or cancer; and
ii) an immunogenic composition comprising at least one vaccine antigen.

131. The method according to claim 130, wherein the binding agent is as defined in any one of claims 1, 83-115.

132. The method according to claim 130 or 131, wherein the immunogenic composition is as defined in any one of claims 1-45.

133. The method according to any one of claims 132-132, wherein the binding agent is administered in further combination with a cytokine such as an interleukin 2 receptor agonist, e.g. human interleukin-2, human interlukin-15 or an analog thereof.

134. The method according to any one of claims 131-133, wherein the binding agent is administered in further combination with an adjuvant, such as an adjuvant as defined in any one of claims 35-44.

135. A binding agent for use in preventing or reducing tumor outgrowth or treatment of cancer in a subject, the binding agent comprising a binding region that binds to CD3 and a binding region that binds to a target on cells of said tumor or cancer, wherein the use comprises providing the antibody to the subject in combination with an immunogenic composition comprising at least one vaccine antigen.

136. The binding agent for use according to claim 135, wherein the binding agent is as defined in any one of claims 1, 95-127.

137. The binding agent for use according to claim 135 or 136, wherein the immunogenic composition is as defined in any one of claims 1-45.

138. The binding agent for use according to any one of claims 135-136, wherein the binding agent is administered in further combination with a cytokine such as an interleukin-2 receptor agonist, e.g. human interleukin-2, human interleukin-15 or an analog thereof.

139. The binding agent for use according to any one of claims 135-136, wherein the binding agent is administered in further combination with an adjuvant, such as an adjuvant as defined in any one of claims 46-5544.

140. An immunogenic composition comprising at least one vaccine antigen for use in preventing or reducing tumor outgrowth or treatment of cancer in a subject, wherein the immunogenic composition is provided to the subject in combination with a binding agent comprising a binding region that binds to CD3 and a binding region that binds to a target on cells of said tumor or cancer.

141. The immunogenic composition for use according to claim 140, wherein the binding agent is as defined in any one of claims 1, 95-127.

142. The immunogenic composition for use according to claim 140 or 141, wherein the immunogenic composition is as defined in any one of claims 1-45.

143. The immunogenic composition for use according to any one of claims 140-142, wherein the immunogenic composition agent is administered in further combination with a cytokine, such as an interleukin-2 receptor agonist, e.g. human interleukin-2, human interleukin-15 or an analog thereof.

144. The immunogenic composition for use according to any one of claims 140-143, wherein the immunogenic composition is administered in further combination with an adjuvant, such as an adjuvant as defined in any one of claims 45-55 or an immunogeneic composition for gene based therapy; e.g. comprisng an immunogenic nucleic acid sequence or a lipid nanoparticle (LNP).

145. Use of a binding agent in the manufacture of a medicament for treatment of cancer in a subject, the binding agent comprising a binding region that binds to CD3 and a binding region that binds to a target on cells of said tumor or cancer, wherein the treatment comprises providing said binding agent to the subject in combination with an immunogenic composition comprising at least one vaccine antigen.

146. Use of the binding agent according to claim 145, wherein the binding agent is as defined in any one of claims 1, 95-127.

147. Use of the binding agent according to claim 145 or 146, wherein the immunogenic composition is as defined in any one of claims 1-45.

148. Use of the abinding agent according to any one of claims 145-146, wherein the binding agent is administered in further combination with a cytokine, such as an interleukin-2 receptor agonist, e.g. human interleukin-2, human interleukin-15 or an analog thereof.

149. Use of the binding agent according to any one of claims 145-148, wherein the binding agent is administered in further combination with an adjuvant, such as an adjuvant as defined in any one of claims 45-55.

150. Use of an immunogenic composition comprising at least one vaccine antigen in the manufacture of a medicament for treatment of cancer in a subject, wherein the immunogenic composition is provided to the subject in combination with a binding agent comprising a binding region that binds to CD3 and a binding region that binds to a target on cells of said tumor or cancer.

151. The immunogenic composition for use according to claim 150, wherein the binding agent is as defined in any one of claims 1, 95-127.

152. The immunogenic composition for use according to claim 150 or 151, wherein the immunogenic composition is as defined in any one of claims 1-45.

153. A kit of parts comprising a binding agent comprising a binding region that binds to CD3 and a binding region that binds to a target on cells of a tumor or cancer, and an immunogenic composition comprising at least one vaccine antigen.

154. The kit of parts according to claim 153, wherein the binding agent is as defined in any one of claims 1, 95-127.

155. The kit of parts according to claim 153 or 154, wherein the immunogenic composition is as defined in any one of claims 1-45.

156. The kit of parts according to any one of claims 153-155, further comprising an adjuvant, such as an adjuvant as defined in any one of claims 45-55.

157. The kit of parts according to any one of claims 153-156, futher comprising an amount of an interleukin-2 receptor agonist, such as human interleukin-2, human interleukin-15 or an analog thereof.

158. The kit of parts according to any one of claims 153-157, further comprising instructions for use, such as for administration of the binding agent in combination with the immunogenic composition.
